(19) United States
(12) Patent Application Publication
(10) Pub. No.: US 2009/0197823 A1
(43) Pub. Date: Aug. 6, 2009

(54) ALISKIREN MODULATION OF NEUROGENESIS

(75) Inventors: Carrolee Barlow, Del Mar, CA (US); Todd A. Carter, San Diego, CA (US); Andrew Morse, San Diego, CA (US); Kai Treuner, San Diego, CA (US); Kym I. Lorrain, San Diego, CA (US)

Correspondence Address: DLA PIPER LLP (US), 4365 EXECUTIVE DRIVE, SUITE 1100, SAN DIEGO, CA, 92121-2133, US

(73) Assignee: BrainCells, Inc., San Diego, CA (US)

(21) Appl. No.: 12/409,431

(22) Filed: Mar. 23, 2009

Related U.S. Application Data
(63) Continuation-in-part of application No. 11/746,539, filed on May 9, 2007.
(60) Provisional application No. 60/746,859, filed on May 9, 2006.
(60) Provisional application No. 61/038,641, filed on Mar. 21, 2008.

(51) Int. Cl. A61K 31/708; A61K 31/165; A61K 31/445; A61K 31/437; C12N 5/06; A61K 31/551; A61P 25/28

(52) U.S. Cl. 514/43; 514/616; 514/317; 514/303; 435/375; 514/218

(57) ABSTRACT

The disclosure provides compositions and methods for treating diseases and conditions of the central and peripheral nervous system by stimulating or increasing neurogenesis by use of a renin inhibitor in combination with one or more neurogenic agents. The disclosure also includes compositions and methods for stimulating or activating the formation of new nerve cells based on the application of a renin inhibitor in combination with one or more neurogenic agents.

### CROSS REFERENCE TO RELATED APPLICATIONS

This application is a Continuation-In-Part application of U.S. Ser. No. 11/746,539, filed May 9, 2007, which claimed priority to U.S. Ser. No. 60/746,859, filed on May 9, 2006, the disclosures of which are hereby incorporated by reference in their entirety for all purposes. This application also claims the benefit under 35 USC § 119(e) to U.S. Application Ser. No. 61/038,641 filed Mar. 21, 2008, the disclosure of which is hereby incorporated by reference in its entirety for all purposes.

### FIELD OF THE DISCLOSURE

The disclosure provides compositions and methods for treating diseases and conditions of the central and peripheral nervous system by stimulating or increasing neurogenesis by use of a renin inhibitor in combination with one or more neurogenic agents. The disclosure also includes compositions and methods for stimulating or activating the formation of new nerve cells based on the application of a renin inhibitor in combination with one or more neurogenic agents.

### BACKGROUND OF THE DISCLOSURE

Neurogenesis is a vital process in the brains of animals and humans, whereby new nerve cells are continuously generated throughout the life span of the organism. The newly born cells are able to differentiate into functional cells of the central nervous system and integrate into existing neural circuits in the brain. The subgranular zone of the hippocampus is one of only two major areas of the adult brain capable of generating new neurons (Gage “Mammalian neural stem cells.” Science 2000 287(5457):1433-8; Warner-Schmidt and Duman “Hippocampal neurogenesis: opposing effects of stress and antidepressant treatment.” Hippocampus 2006 16(3):239-49). In these regions, multipotent neural progenitor cells (NPCs) continue to divide and give rise to new functional neurons and glial cells (Gage “Mammalian neural stem cells.” Science 2000 287(5457):1433-8). Hippocampal neurogenesis is an extremely dynamic process that is regulated by stress, endocrine, and pharmacological factors (Warner-Schmidt and Duman “Hippocampal neurogenesis: opposing effects of stress and antidepressant treatment.” Hippocampus 2006 16(3):239-49). As such, a variety of factors can stimulate adult hippocampal neurogenesis, e.g., adrenalectomy, voluntary exercise, enriched environment and hippocampus dependent learning (Yehuda et al., “Enhanced brain cell proliferation following early adrenalectomy in rats.” J Neorochem 1989 53(1):241-8; van Praag et al. “Running increases cell proliferation and neurogenesis in the adult mouse dentate gyrus.” Nat. Neurosci. 1999 2(3) 266-70; Gould “Serotonin and hippocampal neurogenesis.” Neuropsychopharcology 1999 21(2 suppl):46S-51S; Malberg et al. “Chronic antidepressant treatment increases neurogenesis in adult rat hippocampus.” J Neurosci 2000 20(24):9104-10; Brown et al. “Transient expression of doublecortin during adult neurogenesis.” J Comp Neurol 2003 467(1):1-10; Santarelli et al. “Requirement of hippocampal neurogenesis for the behavioral effects of antidepressants.” Science 2003 301(5634):805-9), whereas other factors, such as adrenal hormones, stress, age and drugs of abuse negatively influence neurogenesis (Cameron and Gould “Adult neurogenesis is regulated by adrenal steroids in the dentate gyrus.” Neurosci 1994 61(2):203-9; Kuhn and Dickinson-Anson “Neurogenesis in the dentate gyrus of the adult rat: age-related decrease of neural progenitor proliferation.” J Neurosci 1996 16(6):2027-33; McEwen “Stress and hippocampal plasticity.” Annu Rev Neurosci 1999 22:105-22; Eisch and Mandyam “Drug dependence and addition, II: Adult neurogenesis and drug abuse.” Am J Psychiatry 2004 161(3):426). Recently, researchers have found that neurogenesis may contribute to the therapeutic effects of drugs used to treat neurological diseases. Therefore, what is need in the art are new drugs that are useful for increasing neuorogenesis.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### BRIEF SUMMARY OF THE DISCLOSURE

The disclosure provides compositions and methods for the prevention and treatment of diseases, disorders, conditions and injuries of the central and peripheral nervous systems by stimulating, increasing or potentiating neurogenesis. Embodiments of the disclosure include methods for treating neurodegenerative disorders, neurological trauma including brain or central nervous system trauma and/or recovery therefrom, depression, anxiety, psychosis, learning and memory disorders and ischemia of the central and/or peripheral nervous systems. In other embodiments, the disclosed compositions and methods are useful for improving cognitive outcomes and mood disorders.

The disclosure also provides methods and compositions for modulating neurogenesis, such as by stimulating, increasing or potentiating neurogenesis. The neurogenesis may be at the level of a cell or tissue. The cell or tissue may be present in an animal subject or more preferably a human subject, or alternatively be in an in vitro or ex vivo setting. In some embodiments, neurogenesis is stimulated or increased in a neural cell or tissue, such as that of the central or peripheral nervous system of an animal or human subject. In cases of an animal or human subject, the methods may be practiced in connection with one or more disease, disorder, or condition of the nervous system as present in the animal or human subject.

Thus, the embodiments disclosed herein include methods for treating a subject suffering from a nervous system disorder, disease, or condition by administering to the subject a therapeutically effective amount of a renin inhibitor in combination with one or more neurogenic agents. In some embodiments, the renin inhibitor is aliskiren or a pharmaceutically acceptable salt, solvate or N-oxide thereof; and the one or more neurogenic agents is a 5-HT₃ receptor antagonist, and/or a PDE inhibitor, and/or an anti-viral agent, and/or a dopamine modulator, and/or a Rho kinase inhibitor and/or an alpha2-adrenergic receptor antagonist. In other embodiments, the renin inhibitor is aliskiren or a pharmaceutically acceptable salt, solvate or N-oxide thereof, and the 5-HT₃ receptor antagonist is azasetron, granisetron, ondansetron; the PDE inhibitor is ibudilast; the antiviral agent is ribavirin; the dopamine modulator is methylphenidate; the Rho kinase inhibitor is fasudil; and the alpha2-adrenergic receptor antagonist is yohimbine, or pharmaceutically acceptable salts, solvates, or N-oxide thereof.

While a renin inhibitor may have some neurogenic activity, it may be advantageous to use it in combination with one or more neurogenic agents as described herein. Of course, the disclosure also includes the use of a renin inhibitor alone. Whether alone or in combination with one or more neurogenic agents, the disclosure may be practiced based on use of a renin inhibitor as a “direct” agent, in that it has direct activity via interaction with its receptor(s) in cells, or as an “indirect” agent in that a renin inhibitor does not directly interact with a receptor. An indirect agent may act on a receptor indirectly, or via production, generation, stability, or retention of an intermediate agent which directly interacts with the receptor.

In additional embodiments, the one or more neurogenic agents as described herein, may be a neurogenic agent that does not act, directly or indirectly, through the same receptor or mechanism as a renin inhibitor. Thus, in some embodiments, a neurogenic agent is one that acts, directly or indirectly, through a mechanism different from that of a renin inhibitor. The one or more neurogenic agents as described herein may be one which acts through a known receptor or one which is known for the treatment of a disease or condition. The disclosure further includes compositions comprising a combination of a renin inhibitor with one or more neurogenic agents as described herein.

In another aspect, the disclosure provides methods for lessening and/or reducing a decline or decrease of cognitive function in a animal or human subject due to a nervous system disorder, disease or condition. In some cases, the method may be applied to maintain and/or stabilize cognitive function in the subject. Thus cognitive impairment may be the result of chronic infection, toxic disorders, neurodegenerative disorders and combinations thereof. In some embodiments disclosed herein, the methods comprise administering a renin inhibitor in combination with one or more neurogenic agents, or pharmaceutically acceptable salts, solvates or N-oxides thereof, to a subject in an amount effective to reduce or lessen cognitive impairment.

In another aspect, the disclosure provides methods for treating a subject suffering from cognitive impairment due to a non-disease state comprising administering to the subject a therapeutically effective amount of a renin inhibitor in combination with one or more neurogenic agents, or pharmaceutically acceptable salts, solvates or N-oxides thereof. Non-limiting examples of non-disease states include cognitive impairment due to aging, chemotherapy and radiation therapy.

In another aspect, the disclosure provides methods for treating a mental disorder with a therapeutically effective amount of a renin inhibitor in combination with one or more neurogenic agents, or pharmaceutically acceptable salts, solvates or N-oxides thereof. In some embodiments, the method may be used to moderate or alleviate the mental disorder in an animal or human subject. Non-limiting examples of a mental disorder include an affective disorder including anxiety and depression. In other embodiments, the method may be used to improve, maintain, or stabilize an affective disorder in a subject.

In another aspect, the disclosed methods include identifying an animal or human subject suffering from one or more diseases, disorders, or conditions, or a symptom thereof, and administering to the subject a therapeutically effective amount of a renin inhibitor in combination with one or more neurogenic agents, or pharmaceutically acceptable salts, solvates or N-oxides thereof. In some embodiments, the disclosed methods include identification of a subject as in need of an increase in neurogenesis; and administering a therapeutically effective amount of renin inhibitor in combination with one or more neurogenic agents. In other embodiments, the subject is a mammal, more preferably a human being.

In another aspect, the disclosure provides methods for stimulating or increasing neurogenesis in a cell or tissue. The cell or tissue is contacted with an effective amount of renin inhibitor in combination with one or more neurogenic agents or a pharmaceutically acceptable salts, solvates or N-oxides thereof to stimulate or increase neurogenesis in said cell or tissue. This cell or tissue may be in an animal or human subject having a condition affecting normal neurogenesis whereby stimulating or increasing neurogenesis improves the condition. Thus the cell or tissue to be treated may exhibit the effects of insufficient amounts of, inadequate levels of, or aberrant neurogenesis. In some embodiments, the subject may be one that has a disease, condition or disorder which results in suppressed or decreased neurogenesis. These subjects would have symptoms and conditions associated with decreased neurogenesis and thus would benefit from a process of stimulating, increasing or potentiating neurogenesis. A non limiting example of such condition is the reduction in or impairment of cognition, such as that due to a chronic infection, a neurodegenerative disease, head injury or a toxic disorder.

In another aspect, the composition of the renin inhibitor in combination with one or more neurogenic agents may be administered to an animal or human subject exhibiting the effects of aberrant neurogenesis. In some embodiments, the aberrant neurogenesis may be attributed to epilepsy, or a condition associated with epilepsy as non-limiting examples. Increased neurogenesis would alleviate the aberrant neurogenic symptoms in the subject.

In an additional aspect, the composition of the renin inhibitor in combination with one or more neurogenic agents may be administered to an animal or human subject that will be subjected to an agent that decreases or inhibits neurogenesis. Non-limiting examples of an inhibitor of neurogenesis include opioid receptor agonists, such as morphine (mu receptor subtype agonist). Non-limiting examples include administering the renin inhibitor in combination with one or more neurogenic agents to a subject before, simultaneously with, or after the subject has be administered morphine or other opiate in connection with a surgical procedure. Other non-limiting embodiments of instances where a subject may be administered the composition of the renin inhibitor in combination with one or more neurogenic agents before, simultaneously with, or after a procedure would include radiation therapy or chemotherapy.

In an additional aspect, the cells undergoing neurogenesis may by neural stem cells (NSCs). These neural stem cells may differentiate along a neuronal lineage, a glial lineage or both. In an additional embodiment of the disclosure the neural stem cells and/or neurogenesis may be in the hippocampus of the subject.

In an additional aspect the composition of the renin inhibitor in combination with one or more neurogenic agents may be used to decrease the level of astrogenesis in a cell or tissue induced by an agent alone. Thus the agent used in combination with the renin inhibitor besides being neurogenic may also be astrogenic. These astrogenic properties may be reduced when used in combination with the renin inhibitor. In an additional embodiment the cell or tissue disclosed may be in an animal or human subject.

In yet another aspect, the disclosure provides methods for modulating neurogenesis, such as by stimulating or increasing neurogenesis, in an animal or human subject by administering the renin inhibitor in combination with one or more neurogenic agents. In some embodiments, the neurogenesis occurs in combination with the stimulation of angiogenesis which provides new cells with access to the circulatory system.

The details of additional embodiments are set forth in the accompanying drawings and the description below. Other features, objects, and advantages of the embodiments will be apparent from the drawings and detailed description, and from the claims.

### BRIEF DESCRIPTION OF THE DRAWINGS

FIG. 1 is a dose-response curve showing the effect of the neurogenic agent aliskiren (renin inhibitor) in combination with azasetron (5-HT₃ receptor antagonist) on neuronal differentiation compared to the effect of either agent alone. When run independently, each compound was tested in a concentration response curve ranging from 0.01 μM to 31.6 μM. In combination, the compounds were combined at equal concentrations at each point (for example, the first point in the combined curve consisted of a test of 0.01 μM aliskiren and 0.01 μM azasetron). Data is presented as the percentage of the neuronal positive control, with basal media values subtracted. When used alone, EC₅₀ was observed at an aliskiren concentration of 8.0 μM or an azasetron concentration of 6.6 μM in test cells. When used in combination, neurogenesis was greatly enhanced: EC₅₀ was observed with the combination of aliskiren and azasetron at concentrations of 1.2 μM each, resulting in a combination index (CI) of 0.36, indicating a synergistic interaction upon neuronal differentiation.

FIG. 2 is a dose-response curve showing effect of the agents, aliskiren (renin inhibitor) and azasetron (5-HT₃ receptor antagonist) in combination, on astrocyte differentiation of human neural stem cells compared to the effect of either agent alone. When run independently, each compound was tested in a concentration response curve ranging from 0.01 μM to 31.6 μM. In combination, the compounds were combined at equal concentrations at each point (for example, the first point in the combined curve consisted of a test of 0.01 μM aliskiren and 0.01 μM azasetron). Data is presented as the percentage of the astrocyte positive control, with basal media values subtracted. When used alone, aliskiren showed no induction of astrocyte formation while azasetron induced astrocyte formation at an extrapolated EC₅₀ of 25.5 μM. When azasetron was used in combination with aliskiren, the EC₅₀ was greater than all tested concentrations (>31.2 μM) and astrocyte differentiation was reduced from a maximum of approximately 23% with azasetron alone to less than 5% with the combination.

FIG. 3 is a dose-response curve showing the effect of the neurogenic agent aliskiren (renin inhibitor) in combination with granisetron (5-HT₃ receptor antagonist) on neuronal differentiation compared to the effect of either agent alone. When run independently, each compound was tested in a concentration response curve ranging from 0.01 μM to 31.6 μM. In combination, the compounds were combined at equal concentrations at each point (for example, the first point in the combined curve consisted of a test of 0.01 μM aliskiren and 0.01 μM granisetron). Data is presented as the percentage of the neuronal positive control, with basal media values subtracted. When used alone, EC₅₀ was observed at an aliskiren concentration of 8.0 μM or a granisetron concentration of 6.7 μM in test cells. When used in combination, neurogenesis was greatly enhanced: EC₅₀ was observed with the combination of aliskiren and granisetron at concentrations of 0.62 μM each, resulting in a combination index (CI) of 0.18, indicating a synergistic interaction upon neuronal differentiation.

FIG. 4 is a dose-response curve showing the effect of the agents, aliskiren (renin inhibitor) and granisetron (5-HT₃ receptor antagonist) in combination, on astrocyte differentiation of human neural stem cells compared to the effect of either agent alone. When run independently, each compound was tested in a concentration response curve ranging from 0.01 μM to 31.6 μM. In combination, the compounds were combined at equal concentrations at each point (for example, the first point in the combined curve consisted of a test of 0.01 μM aliskiren and 0.01 μM granisetron). Data is presented as the percentage of the astrocyte positive control, with basal media values subtracted. When used alone, aliskiren showed no induction of astrocyte formation and granisetron induced astrocyte formation at an extrapolated EC₅₀ of 23.4 μM. When granisetron was used in combination with aliskiren, the EC₅₀ was greater than all tested concentrations (>31.2 μM) and astrocyte differentiation was reduced from a maximum of approximately 38% with granisetron alone to less than 5% with the combination.

FIG. 5 is a dose-response curve showing the effect of the neurogenic agent aliskiren (renin inhibitor) in combination with ondansetron (5-HT₃ receptor antagonist) on neuronal differentiation compared to the effect of either agent alone. When run independently, each compound was tested in a concentration response curve ranging from 0.01 μM to 31.6 μM. In combination, the compounds were combined at equal concentrations at each point (for example, the first point in the combined curve consisted of a test of 0.01 μM aliskiren and 0.01 μM ondansetron). Data is presented as the percentage of the neuronal positive control, with basal media values subtracted. When used alone, EC₅₀ was observed at an aliskiren concentration of 8.0 μM or an ondansetron concentration of 4.2 μM in test cells. When used in combination, neurogenesis was greatly enhanced: EC₅₀ was observed with the combination of aliskiren and ondansetron at concentrations of 0.74 μM each, resulting in a combination index of 0.28, indicating a synergistic interaction upon neuronal differentiation.

FIG. 6 is a dose-response curve showing the effect of the agents aliskiren (renin inhibitor) and ondansetron (5-HT₃ receptor antagonist) in combination on astrocyte differentiation of human neural stem cells compared to the effect of either agent alone. When run independently, each compound was tested in a concentration response curve ranging from 0.01 μM to 31.6 μM. In combination, the compounds were combined at equal concentrations at each point (for example, the first point in the combined curve consisted of a test of 0.01 μM ondansetron and 0.01 μM ondansetron). Data is presented as the percentage of the astrocyte positive control, with basal media values subtracted. When used alone, aliskiren showed no induction of astrocyte formation and ondansetron induced astrocyte formation at an extrapolated EC₅₀ of 27.5 μM. When ondansetron was used in combination with aliskiren, the EC₅₀ was greater than all tested concentrations (>31.2 μM) and astrocyte differentiation was reduced from a maximum of approximately 22% with ondansetron alone to less than 5% with the combination.

FIG. 7 is a dose-response curve showing the effect of the neurogenic agent aliskiren (renin inhibitor) in combination with ribavirin (anti-viral agent) on neuronal differentiation compared to the effect of either agent alone. When run independently, each compound was tested in a concentration response curve ranging from 0.01 μM to 31.6 μM. In combination, the compounds were combined at equal concentrations at each point (for example, the first point in the combined curve consisted of a test of 0.01 μM aliskiren and 0.01 μM ribavirin). Data is presented as the percentage of the neuronal positive control, with basal media values subtracted. When used alone, EC₅₀ was observed at an aliskiren concentration of 11.1 μM or a ribavirin concentration of 10.9 μM in test cells. When used in combination, neurogenesis was greatly enhanced: EC₅₀ was observed with the combination of aliskiren and ribavirin at concentrations of 0.51 μM each, resulting in a combination index of 0.10, indicating a synergistic interaction upon neuronal differentiation.

FIG. 8 is a dose-response curve showing effect of the agents aliskiren (renin inhibitor) and ribavirin (anti-viral agent) in combination on astrocyte differentiation of human neural stem cells compared to the effect of either agent alone. When run independently, each compound was tested in a concentration response curve ranging from 0.01 μM to 31.6 μM. In combination, the compounds were combined at equal concentrations at each point (for example, the first point in the combined curve consisted of a test of 0.01 μM aliskiren and 0.01 μM ribavirin). Data is presented as the percentage of the astrocyte positive control, with basal media values subtracted. When used alone, aliskiren showed no induction of astrocyte formation and ribavirin induced astrocyte formation at an extrapolated EC₅₀ of 20.6 μM. When ribavirin was used in combination with aliskiren, the EC₅₀ was greater than all tested concentrations (>31.2 μM) and astrocyte differentiation was reduced from a maximum of approximately 22% with ribavirin alone to approximately 5% with the combination.

FIG. 9 is a dose-response curve showing effect of the neurogenic agent aliskiren (renin inhibitor) in combination with methylphenidate (dopamine modulator) on neuronal differentiation compared to the effect of either agent alone. When run independently, each compound was tested in a concentration response curve ranging from 0.01 μM to 31.6 μM. In combination, the compounds were combined at equal concentrations at each point (for example, the first point in the combined curve consisted of a test of 0.01 μM aliskiren and 0.01 μM methylphenidate). Data is presented as the percentage of the neuronal positive control, with basal media values subtracted. When used alone, EC₅₀ was observed at an aliskiren concentration of 11.1 μM or a methylphenidate concentration of 2.2 μM in test cells. When used in combination, neurogenesis was greatly enhanced: EC₅₀ was observed with the combination of aliskiren and methylphenidate at concentrations of 0.54 μM each, resulting in a combination index of 0.13, indicating a synergistic interaction upon neuronal differentiation.

FIG. 10 is a dose-response curve showing effect of the neurogenic agent aliskiren (renin inhibitor) in combination with yohimbine (α2-adrenergic receptor antagonist) on neuronal differentiation compared to the effect of either agent alone. When run independently, each compound was tested in a concentration response curve ranging from 0.01 μM to 31.6 μM. In combination, the compounds were combined at equal concentrations at each point (for example, the first point in the combined curve consisted of a test of 0.01 μM aliskiren and 0.01 μM yohimbine). Data is presented as the percentage of the neuronal positive control, with basal media values subtracted. When used alone, EC₅₀ was observed at an aliskiren concentration of 11.1 μM or a yohimbine concentration of 12.0 μM in test cells. When used in combination, neurogenesis was greatly enhanced: EC₅₀ was observed with the combination of aliskiren and yohimbine at concentrations of 1.7 μM each, resulting in a combination index of 0.31, indicating a synergistic interaction upon neuronal differentiation.

FIG. 11 is a dose-response curve showing effect of the neurogenic agent aliskiren (renin inhibitor) in combination with fasudil (Rho kinase inhibitor) on neuronal differentiation compared to the effect of either agent alone. When run independently, each compound was tested in a concentration response curve ranging from 0.01 μM to 31.6 μM. In combination, the compounds were combined at equal concentrations at each point (for example, the first point in the combined curve consisted of a test of 0.01 μM aliskiren and 0.01 μM fasudil). Data is presented as the percentage of the neuronal positive control, with basal media values subtracted. When used alone, EC₅₀ was observed at an aliskiren concentration of 11.1 μM or a fasudil concentration of 1.0 μM in test cells. When used in combination, neurogenesis was greatly enhanced: EC₅₀ was observed with the combination of aliskiren and fasudil at concentrations of 0.57 μM each, resulting in a combination index of 0.65, indicating a synergistic interaction upon neuronal differentiation.

FIG. 12 is a dose-response curve showing effect of the neurogenic agents a aliskiren (renin inhibitor) and ibudilast (PDE inhibitor) in combination on neuronal differentiation of human neural stem cells compared to the effect of either agent alone. When run independently, aliskiren was tested in a concentration response curve ranging from 0.01 μM to 31.6 μM, and ibudilast was tested in a response curve ranging from 0.003-10.0 μM. In combination, the compounds were combined at about 3.2:1 ratio at each point (for example, the first point in the combined curve consisted of a test of 0.01 μM aliskiren and 0.003 μM ibudilast). Data is presented as the percentage of the neuronal positive control, with basal media values subtracted. When used alone, EC₅₀ was observed at an aliskiren concentration of 5.2 μM or an ibudilast concentration of 0.28 μM in test cells. When used in combination, neurogenesis is greatly enhanced: EC₅₀ was observed with the combination of aliskiren and ibudilast at concentrations of 0.24 μM and 0.09 μM, respectively, resulting in a synergistic combination index of 0.38.

### DETAILED DESCRIPTION OF THE DISCLOSURE

As used herein, the term “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. Preferred alkyl groups have 1-8 carbons. “Alkenyl” and other like terms include carbon chains containing at least one unsaturated carbon-carbon bond. “Alkynyl” and other like terms include carbon chains containing at least one carbon-carbon triple bond.

As used herein, the term “cycloalkyl” means carbocycles containing no heteroatoms, and includes mono-, bi- and tricyclic saturated carbocycles, as well as fused ring systems. Examples of cycloalkyl include but are not limited today cyclopropyl, cyclobutyl, cyclopentyl, cyclohexyl, cycloheptyl, decahydronaphthalene, adamantyl, indanyl, indenyl, fluorenyl, 1,2,3,4-tetrahydronaphthalene and the like.

As used herein, the term “aryl” means an aromatic substituent that is a single ring or multiple rings fused together. Exemplary aryl groups include, without limitation, phenyl, naphthyl, anthracenyl, pyridinyl, pyrazinyl, pyrimidinyl, triazinyl, thiophenyl, furanyl, pyrrolyl, oxazolyl, isoxazolyl, imidazolyl, thioimidazolyl, oxazolyl, isoxazolyl, triazyolyl, and tetrazolyl groups. Aryl groups that contain one or more heteroatoms (e.g., pyridinyl) are often referred to as “heteroaryl groups.” When formed of multiple rings, at least one of the constituent rings is aromatic. In some embodiments, at least one of the multiple rings contain a heteroatom, thereby forming heteroatom-containing aryl groups. Heteroatom-containing aryl groups include, without limitation, benzoxazolyl, benzimidazolyl, quinoxalinyl, benzofuranyl, indolyl, indazolyl, benzimidazolyl, quinolinoyl, and 1H-benzo[d][1,2,3]triazolyl groups and the like. Heteroatom-containing aryl groups also include aromatic rings fused to a heterocyclic ring comprising at least one heteroatom and at least one carbonyl group. Such groups include, without limitation, dioxo tetrahydroquinoxalinyl and dioxo tetrahydroquinazolinyl groups.

As used herein, the term “arylalkoxy” means an aryl group bonded to an alkoxy group.

As used herein, the term “arylamidoalkyl” means an aryl-C(O)NR-alkyl or aryl-NRC(O)-alkyl.

As used herein, the term “arylalkylamidoalkyl” means an aryl-alkyl-C(O)NR-alkyl or aryl-alkyl-NRC(O)-alkyl, wherein R is any suitable group listed below.

As used herein, the term “arylalkyl” refers to an aryl group bonded to an alkyl group.

As used herein, the term “halogen” or “halo” refers to chlorine, bromine, fluorine or iodine.

As used herein, the term “haloalkyl” means an alkyl group having one or more halogen atoms (e.g., trifluoromethyl).

As used herein, the term “heteroalkyl” refers to an alkyl moiety which comprises a heteroatom such as N, O, P, B, S, or Si. The heteroatom may be connected to the rest of the heteroalkyl moiety by a saturated or unsaturated bond. Thus, an alkyl substituted with a group, such as heterocycloalkyl, substituted heterocycloalkyl, heteroaryl, substituted heteroaryl, alkoxy, aryloxy, boryl, phosphino, amino, silyl, thio, or seleno, is within the scope of the term heteroalkyl. Examples of heteroalkyls include, but are not limited to, cyano, benzoyl, and substituted heteroaryl groups. For example, 2-pyridyl, 3-pyridyl, 4-pyridyl, and 2-furyl, 3-furyl, 4-furyl, 2-imidazolyl, 3-imidazolyl, 4-imidazolyl, 5-imidazolyl.

As used herein, the term “heteroarylalkyl” means a heteroaryl group to which an alkyl group is attached.

As used herein, the term “heterocycle” means a monocyclic or polycyclic ring comprising carbon and hydrogen atoms, optionally having 1, 2 or more multiple bonds, and the ring atoms contain at least one heteroatom, specifically 1 to 4 heteroatoms, independently selected from nitrogen, oxygen, and sulfur. Heterocycle ring structures include, but are not limited to, mono-, bi-, and tri-cyclic compounds. Specific heterocycles are monocyclic or bicyclic. Representative heterocycles include cyclic ureas, morpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrazolyl, azabicyclo[3.2.1]octanyl, hexahydro-1H-quinolizinyl, and urazolyl. A heterocyclic ring may be unsubstituted or substituted.

As used herein, the term “heterocycloalkyl” refers to a cycloalkyl group in which at least one of the carbon atoms in the ring is replaced by a heteroatom (e.g., O, S or N).

As used herein, the term “heterocycloalkylalkyl” means a heterocycloalkyl group to which the an alkyl group is attached.

As used herein, the term “substituted” specifically envisions and allows for one or more substitutions that are common in the art. However, it is generally understood by those skilled in the art that the substituents should be selected so as to not adversely affect the useful characteristics of the compound or adversely interfere with its function. Suitable substituents may include, for example, halogen groups, perfluoroalkyl groups, perfluoroalkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, arylalkyl or heteroarylalkyl groups, arylalkoxy or heteroarylalkoxy groups, amino groups, alkyl- and dialkylamino groups, carbamoyl groups, alkylcarbonyl groups, carboxyl groups, alkoxycarbonyl groups, alkylaminocarbonyl groups, dialkylamino carbonyl groups, arylcarbonyl groups, aryloxycarbonyl groups, alkylsulfonyl groups, arylsulfonyl groups, cycloalkyl groups, cyano groups, C₁-C₆ alkylthio groups, arylthio groups, nitro groups, keto groups, acyl groups, boronate or boronyl groups, phosphate or phosphonyl groups, sulfamyl groups, sulfonyl groups, sulfinyl groups, and combinations thereof. In the case of substituted combinations, such as “substituted arylalkyl,” either the aryl or the alkyl group may be substituted, or both the aryl and the alkyl groups may be substituted with one or more substituents. Additionally, in some cases, suitable substituents may combine to form one or more rings as known to those of skill in the art.

The compounds described herein may contain one or more double bonds and may thus give rise to cis/trans isomers as well as other conformational isomers. The present disclosure includes all such possible isomers as well as mixtures of such “isomers”.

The compounds described herein, and particularly the substituents described above, may also contain one or more asymmetric centers and may thus give rise to diastereomers and optical isomers. The present disclosure includes all such possible diastereomers as well as their racemic mixtures, their substantially pure resolved enantiomers, all possible geometric isomers, and acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

As used herein, the term “salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric with replacement of one or both protons, sulfamic, phosphoric with replacement of one or both protons, e.g. orthophosphoric, or metaphosphoric, or pyrophosphoric and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, embonic, nicotinic, isonicotinic and amino acid salts, cyclamate salts, fumaric, toluenesulfonic, methanesulfonic, N-substituted sulphamic, ethane disulfonic, oxalic, and isethionic, and the like. Also, such conventional non-toxic salts include those derived from inorganic acids such as non toxic metals derived from group Ia, Ib, IIa and IIb in the periodic table. For example, lithium, sodium, or potassium magnesium, calcium, zinc salts, or ammonium salts such as those derived from mono, di and trialkyl amines. For example methyl-, ethyl-, diethyl, triethyl, ethanol, diethanol- or triethanol amines or quaternary ammonium hydroxides.

The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile. Lists of suitable salts are found in Remington's Pharmaceutical Sciences, 17th ed., Mack Publishing Company, Easton, Pa., 1985, p. 1418, the disclosure of which is hereby incorporated by reference.

As used herein, the term “solvate” means a compound, or a salt thereof, that further includes a stoichiometric or non-stoichiometric amount of solvent bound by non-covalent intermolecular forces. Where the solvent is water, the solvate is a hydrate.

As used herein, the term “analog thereof” in the context of the compounds disclosed herein includes diastereomers, hydrates, solvates, salts, prodrugs, and N-oxides of the compounds.

“Neurogenesis” is defined herein as proliferation, differentiation, migration and/or survival of a neural cell in vivo or in vitro. In some embodiments, the neural cell is an adult, fetal, or embryonic neural stem cell or population of cells. The cells may be located in the central nervous system or elsewhere in an animal or human being. The cells may also be in a tissue, such as neural tissue. In some embodiments, the neural cell is an adult, fetal, or embryonic progenitor cell or population of cells, or a population of cells comprising a mixture of stem cells and progenitor cells. Neural cells include all brain stem cells, all brain progenitor cells, and all brain precursor cells. Neurogenesis includes neurogenesis as it occurs during normal development, as well as neural regeneration that occurs following disease, damage or therapeutic intervention, such as by the treatment described herein.

As used herein, a “neurogenic agent” is defined as a chemical agent or reagent that can promote, stimulate, or otherwise increase the amount or degree or nature of neurogenesis in vivo or ex vivo or in vitro relative to the amount, degree, or nature of neurogenesis in the absence of the agent or reagent. In some embodiments, treatment with a neurogenic agent increases neurogenesis if it promotes neurogenesis by at least about 5%, at least about 10%, at least about 25%, at least about 50%, at least about 100%, at least about 500%, or more in comparison to the amount, degree, and/or nature of neurogenesis in the absence of the agent, under the conditions of the method used to detect or determine neurogenesis.

As used herein, the term “neurogenic combination of a renin inhibitor and one or more neurogenic agents” refers to a combination of neurogenesis modulating agents. In some embodiments, administering a neurogenic, or neuromodulating, combination according to methods provided herein modulates neurogenesis in a target tissue and/or cell-type by at least about 50%, at least about 75%, or at least about 90% or more in comparison to the absence of the combination. In further embodiments, neurogenesis is modulated by at least about 95% or by at least about 99% or more.

As used herein, a “neuromodulating combination” may be used to inhibit a neural cell's proliferation, division, or progress through the cell cycle. Alternatively, a neuromodulating combination may be used to stimulate survival and/or differentiation in a neural cell. As an additional alternative, a neuromodulating combination may be used to inhibit, reduce, or prevent astrocyte activation and/or astrogenesis or astrocyte differentiation.

The term “astrogenic” is defined in relation to “astrogenesis” which refers to the activation, proliferation, differentiation, migration and/or survival of an astrocytic cell in vivo or in vitro. Non-limiting examples of astrocytic cells include astrocytes, activated microglial cells, astrocyte precursors and potentiated cells, and astrocyte progenitor and derived cells. In some embodiments, the astrocyte is an adult, fetal, or embryonic astrocyte or population of astrocytes. The astrocytes may be located in the central nervous system or elsewhere in an animal or human being. The astrocytes may also be in a tissue, such as neural tissue. In some embodiments, the astrocyte is an adult, fetal, or embryonic progenitor cell or population of cells, or a population of cells comprising a mixture of stem and/or progenitor cells, which is/are capable of developing into astrocytes. Astrogenesis includes the proliferation and/or differentiation of astrocytes as it occurs during normal development, as well as astrogenesis that occur following disease, damage or therapeutic intervention.

The term “stem cell” (or neural stem cell (NSC)), as used herein, refers to an undifferentiated cell that is capable of self-renewal and differentiation into neurons, astrocytes, and/or oligodendrocytes.

The term “progenitor cell” (e.g., neural progenitor cell), as used herein, refers to a cell derived from a stem cell that is not itself a stem cell. Some progenitor cells can produce progeny that are capable of differentiating into more than one cell type.

The terms “animal subject” refers to a non-human mammals, such as a primate, canine, or feline. In other embodiments, the terms refer to an animal that is domesticated (e.g. livestock) or otherwise subject to human care and/or maintenance (e.g. zoo animals and other animals for exhibition). In other non-limiting examples, the terms refer to ruminants or carnivores, such as dogs, cats, birds, horses, cattle, sheep, goats, marine animals and mammals, penguins, deer, elk, and foxes.

The term “condition” refers to the physical and/or psychological state of an animal or human subject selected for treatment with the disclosed compound or compounds. The physical and/or psychological state of the animal or human subject at the time of treatment may include but is not limited to a disease state, a disease symptom, and/or a disease syndrome. The physical and/or psychological state of the animal or human subject may be the result of an injury, disease or disorder and/or a result of treating such injury, disease or disorder.

The term “nervous system disorder” refers to diseases and disorders of the nervous system categorized under “mental disorders” or “diseases and disorders of the central nervous system”.

The term “mental disorder” refers to a group of disorders that are commonly associated with an anxiety disorder, a mood disorder or schizophrenia as disclosed in “Harrison's Principles of Internal Medicine” 17^{th} edition, which is herein incorporated in its entirety.

The term “affective disorder” as used herein encompasses depression and anxiety. An “affective disorder” comprises the symptoms of depression and/or anxiety.

The term “anxiety disorder” refers to or connotes significant distress and dysfunction due to feelings of apprehension, guilt, fear, and the like. Anxiety disorders include, but are not limited to panic disorders, posttraumatic stress disorder, obsessive-compulsive disorder and phobic disorders.

The term “mood disorder” is typically characterized by pervasive, prolonged, and disabling exaggerations of mood, which are associated with behavioral, physiologic, cognitive, neurochemical and psychomotor dysfunctions. As used herein a mood disorder includes but is not limited to bipolar disorders, depression including major depressive disorder, and depression associated with various disease states and injuries.

The term “diseases and disorders of the central nervous system” include but are not limited to epilepsy, cerebrovascular disease, cognitive impairment, neuropathy, myelopathy and head injury as disclosed in “Harrison's Principles of Internal Medicine” 17^{th} edition, which is incorporated in its entirety.

As used herein, the term “neurodegenerative disorder” encompasses diseases and disorders of the central nervous system wherein neuronal perturbations are the result of the disease or disorder. As non-limiting examples of neuronal perturbations are those noted within the hippocampus resulting in decreased neurogenesis, aberrant neurogenesis, as well as defects to neuronal and synaptic plasticity.

As used herein, the term “cognitive impairment” refers to diminished or reduced cognitive function. This may be the result of a number of natural and physical events including but not limited to aging, head trauma, diseases and disorders of the central nervous system, therapies related to treating a disease or disorder (drugs, chemotherapy and radiation therapy), as well as alcohol and drug abuse.

The term “cognitive function” refers to high-level brain functions of an animal or human subject relating to information gathering and/or processing; the understanding, reasoning, and/or application of information and/or ideas; the abstraction or specification of ideas and/or information; acts of creativity, problem-solving, and possibly intuition; and mental processes such as learning, perception, and/or awareness of ideas and/or information. The mental processes are distinct from those of beliefs, desires, and the like. In some embodiments, cognitive function may be assessed, and thus optionally defined, via one or more tests or assays for cognitive function. Non-limiting examples of a test or assay for cognitive function include CANTAB (see for example Fray et al. “CANTAB battery: proposed utility in neurotoxicology.” Neurotoxicol Teratol. 1996; 18(4):499-504), Stroop Test, Trail Making, Wechsler Digit Span, or the CogState computerized cognitive test (see also Dehaene et al. “Reward-dependent learning in neuronal networks for planning and decision making.” Prog Brain Res. 2000; 126:217-29; Iverson et al. “Interpreting change on the WAIS-III/WMS-III in clinical samples.” Arch Clin Neuropsychol. 2001 16(2):183-91; and Weaver et al. “Mild memory impairment in healthy older adults is distinct from normal aging.” Brain Cogn. 2006; 60(2): 146-55). The novel object recognition assay as used herein is a model used for screening potential compounds having an effect on cognitive function.

The term “dementia” is the progressive decline in cognitive function due to damage or disease in the body beyond what might be expected from normal aging. Dementia is a non-specific illness syndrome in which affected areas of cognition may be memory, attention, language, and problem solving.

As used herein, “treating” includes prevention, amelioration, alleviation, and/or elimination of the disease, disorder, or condition being treated or one or more symptoms of the disease, disorder, or condition being treated, as well as improvement in the overall well being of a patient, as measured by objective and/or subjective criteria. In some embodiments, treating is used for reversing, attenuating, minimizing, suppressing, or halting undesirable or deleterious effects of, or effects from the progression of, a disease, disorder, or condition of the central and/or peripheral nervous systems. In other embodiments, the method of treating may be advantageously used in cases where additional neurogenesis would replace, replenish, or increase the numbers of cells lost due to injury or disease as non-limiting examples.

As used herein, “IC₅₀” and “EC₅₀” values are concentrations of an agent, in a combination of a renin inhibitor in combination with one or more neurogenic agents, which reduce and promote, respectively, neurogenesis or another physiological activity (e.g., the activity of a receptor) to a half-maximal level. IC₅₀ and EC₅₀ values may be assayed in a variety of environments, including cell-free environments, cellular environments (e.g., cell culture assays), multicellular environments (e.g., in tissues or other multicellular structures), and/or in vivo. In some embodiments, one or more neurogenesis modulating agents in a combination or method disclosed herein individually have IC₅₀ or EC₅₀ values of less than about 10 μM, less than about 1 μM, or less than about 0.1 μM or lower. In other embodiments, an agent in a combination has an IC₅₀ of less than about 50 nM, less than about 10 nM, or less than about 1 nM or lower.

In some embodiments disclosed herein, the selectivity of one or more agents, in a combination of a renin inhibitor in combination with one or more neurogenic agents, is individually measured as the ratio of the IC₅₀ or EC₅₀ value for a desired effect (e.g., modulation of neurogenesis) relative to the IC₅₀/EC₅₀ value for an undesired effect. In some embodiments, a “selective” agent in a combination has a selectivity of less than about 1:2, less than about 1:10, less than about 1:50, or less than about 1:100. In some embodiments, one or more agents in a combination individually exhibits selective activity in one or more organs, tissues, and/or cell types relative to another organ, tissue, and/or cell type. For example, in some embodiments, an agent in a combination selectively modulates neurogenesis in a neurogenic region of the brain, such as the hippocampus (e.g., the dentate gyrus), the subventricular zone, and/or the olfactory bulb.

In other embodiments disclosed herein, modulation by a combination of agents is in a region containing neural cells affected by disease or injury, a region containing neural cells associated with disease effects or processes, or a region containing neural cells affected by another event injurious to neural cells. Non-limiting examples of such events include stroke, radiation therapy or chemotherapy. In additional embodiments, a neuromodulating combination substantially modulates two or more physiological activities or target molecules, while being substantially inactive against one or more other molecules and/or activities

In some cases, administration of rennin inhibitor in combination with a neurogenic agent, results in improved efficacy, fewer side effects, lower effective dosages, less frequent dosing, and/or other desirable effects than either agent alone.

The methods described herein may be used to treat any disease or condition for which it is beneficial to promote or otherwise stimulate or increase neurogenesis. One focus of the methods described herein is to achieve a therapeutic result by stimulating, increasing or potentiating neurogenesis via use of a renin inhibitor in combination with one or more neurogenic agents. In some embodiments, the renin inhibitor is aliskiren or a pharmaceutically acceptable salt, solvate or N-oxide thereof; and the one or more neurogenic agents is a 5-HT₃ receptor antagonist, a PDE inhibitor, an anti-viral agent, a dopamine modulator, a Rho kinase inhibitor an alpha2-adrenergic receptor antagonist and combinations thereof. In other embodiments, the renin inhibitor is aliskiren or a pharmaceutically acceptable salt, solvate or N-oxide thereof, and the 5-HT₃ receptor antagonist is azasetron, granisetron, or ondansetron; the PDE inhibitor is ibudilast; the antiviral agent is ribavirin; the dopamine modulator is methylphenidate; the Rho kinase inhibitor is fasudil; and the alpha2-adrenergic receptor antagonist is yohimbine, or pharmaceutically acceptable salt, solvate or N-oxide thereof. Thus, certain methods described herein may be used to treat any disease or condition susceptible to treatment by increasing neurogenesis.

Within the scope of the disclosure are methods applied to modulating neurogenesis in vivo, in vitro, or ex vivo. In in vivo embodiments, the cells may be present in a tissue or organ of a subject animal or human being. Non-limiting examples of cells include those capable of neurogenesis, such as to result, whether by differentiation or by a combination of differentiation and proliferation, in differentiated neural cells. As described herein, neurogenesis includes the differentiation of neural cells along different potential lineages. In some embodiments, the differentiation of neural stem or progenitor cells is along a neuronal cell lineage to produce neurons. In other embodiments, the differentiation is along both neuronal and glial cell lineages.

In additional embodiments, the disclosure further includes differentiation along a neuronal cell lineage to the exclusion of one or more cell types in a glial cell lineage. Non-limiting examples of glial cell types include oligodendrocytes and radial glial cells, as well as astrocytes, which have been reported as being of an “astroglial lineage.” Therefore, embodiments of the disclosure include differentiation along a neuronal cell lineage to the exclusion of one or more cell types selected from oligodendrocytes, radial glial cells, and astrocytes.

In further embodiments, the methods described herein may allow for treatment of diseases characterized by pain, addiction, and/or depression by directly replenishing, replacing, and/or supplementing neurons and/or glial cells. In further embodiments, the methods described herein may enhance the growth and/or survival of existing neural cells, and/or slow or reverse the loss of such cells in a neurodegenerative condition.

Where a method comprises contacting a neural cell with a renin inhibitor in combination with one or more neurogenic agents, the result may be an increase in neuro-differentiation. The method may be used to potentiate a neural cell for proliferation, and thus neurogenesis, via administration of a renin inhibitor in combination with one or more neurogenic agents. Thus, the disclosure includes methods for maintaining, stabilizing, stimulating increasing or potentiating neurodifferentiation in a cell or tissue by use of a renin inhibitor in combination with one or more neurogenic agents. The method may comprise contacting a cell or tissue with a renin inhibitor in combination with one or more neurogenic agents, to maintain, stabilize stimulate, increase or potentiate neurodifferentiation in the cell or tissue.

In another aspect, the disclosure provides methods for stimulating or increasing neurogenesis in a cell or tissue, by contacting the cell or tissue with a renin inhibitor in combination with one or more neurogenic agents, wherein the effect is to produce neurogenesis in the cell or tissue, and wherein the neurogenesis comprises differentiation of neural stem cells (NSCs) along a neuronal lineage or along a glial cell line.

The disclosure also includes methods comprising contacting the cell or tissue with a renin inhibitor in combination with one or more neurogenic agents, which stimulates or increases proliferation or cell division in a neural cell. The increase in neuroproliferation may be due to the composition of a renin inhibitor in combination with one or more neurogenic agents. In some cases, the disclosed methods comprising a renin inhibitor in combination with one or more neurogenic agents, may be used to produce neurogenesis (in this case both neurodifferentiation and/or proliferation) in a population of neural cells. In some embodiments, the cell or tissue is in an animal subject or a human patient. Non-limiting examples of conditions in need of neurogenesis include a human patient treated with chemotherapy and/or radiation, or other therapy or condition which is detrimental to cognitive function; or a human patient diagnosed with a degenerative disease; or a human patient diagnosed as having epilepsy, a condition associated with epilepsy, or seizures associated with epilepsy.

In another aspect, the disclosure provides methods for stimulating or increasing neurogenesis in a cell or tissue, by contacting the cell or tissue with a renin inhibitor in combination with one or more neurogenic agents, wherein the effect is to produce neurogenesis in the cell or tissue, wherein the cell or tissue is in an animal subject or a human patient, and wherein the subject or patient has one or more chemical addiction or dependency.

In another aspect, the disclosure provides methods for treating a nervous system disorder related to a mental disorder or a disease or disorder of the central nervous system in a subject or patient, the method comprising administering a renin inhibitor in combination with one or more neurogenic agents to the subject or patient, wherein the effect is to produce an improvement in the disorder or disease in the subject or patient.

In another aspect, the disease or disorder of the central nervous system to be treated is selected from epilepsy, cerebrovascular ischemia, cognitive impairment, neuropathy, myelopathy head injury or other neurologically related disorder.

In another aspect, the disclosed methods may be used to moderate or alleviate a mental disorder in a subject or patient. The mental disorder to be treated may be selected from an affective disorder or schizophrenia. As used herein an affective disorder encompasses anxiety and depression. Thus, the disclosure includes methods for treating a mental disorder in a subject or patient by administering a therapeutically effective amount of a renin inhibitor in combination with one or more neurogenic agents. Non-limiting examples of the method include the administration of a renin inhibitor in combination with one or more neurogenic agents to a subject of patient that is under a treatment and/or has a condition that results in a mental disorder.

In some embodiments, wherein the subject or patient demonstrates impaired cognitive function, methods of the disclosure may be useful for enhancing or improving the cognitive impairment. The methods may comprise administering a renin inhibitor in combination with one or more neurogenic agents to a subject or patient to enhance or improve a decline or decrease of cognitive function due to a therapy and/or condition that reduces cognitive function. Non-limiting examples and conditions affecting cognitive impairment are aging, chronic infections, toxic disorders, degenerative disorders or combinations thereof. Cognitive impairment due to age may be age-associated memory impairment (AAMI), age-associated cognitive decline (AACD), mild cognitive impairment or age-related memory loss. Non-limiting examples of chronic infections affecting cognition are HIV or Creutzfeldt-Jakob disease. Non-limiting examples of toxic disorders affecting cognition are radiation therapy, chemotherapy, drug or alcohol abuse and combinations thereof. Non-limiting examples of degenerative disorders affecting cognition are Alzheimer's disease, Huntington's disease, Parkinson's disease, Multiple Sclerosis and combinations thereof.

Other methods of the disclosure include treatment to affect or maintain the cognitive function of a subject or patient. In some embodiments, the maintenance or stabilization of cognitive function may be at a level, or thereabouts, present in a subject or patient in the absence of a therapy and/or condition that reduces cognitive function. In alternative embodiments, the maintenance or stabilization may be at a level, or thereabouts, present in a subject or patient as a result of a therapy and/or condition that reduces cognitive function.

The disclosed methods may optionally include assessing or measuring cognitive function of the subject or patient before, during, and/or after administration of the treatment to detect or determine the effect thereof on cognitive function. In one embodiment, the disclosed methods may comprise i) treating a subject or patient that has been previously assessed for cognitive function and ii) reassessing cognitive function in the subject or patient during or after the course of treatment. The assessment may measure cognitive function for comparison to a control or standard value (or range) in subjects or patients in the absence of a renin inhibitor in combination with one or more neurogenic agents. This may be used to assess the efficacy of a renin inhibitor in combination with one or more neurogenic agents in alleviating the reduction in cognitive function.

In another aspect, the disclosure provides methods for stimulating or increasing neurogenesis in a cell or tissue, by contacting the cell or tissue with a renin inhibitor in combination with one or more neurogenic agents, wherein the effect is to produce neurogenesis in the cell or tissue, wherein a renin inhibitor in combination with one or more neurogenic agents is in a pharmaceutically acceptable formulation.

The disclosure includes methods for the identification of an individual suffering from one or more disease, disorder, or condition, or a symptom thereof, and administering to the subject or patient a therapeutically effective amount of a renin inhibitor in combination with one or more neurogenic agents. The identification of a subject or patient as having one or more disease, disorder or condition, or a symptom thereof, may be made by a skilled practitioner using any appropriate means known in the field.

In some embodiments, the identification of a patient in need of neurogenic modulation comprises identifying a patient who has or will be exposed to a factor or condition known to inhibit neurogenesis, including but not limited to, stress, aging, sleep deprivation, hormonal changes (e.g., those associated with puberty, pregnancy), or aging (e.g., menopause), lack of exercise, lack of environmental stimuli (e.g., social isolation), diabetes and drugs of abuse (e.g., alcohol, especially chronic use; opiates and opioids; psychostimulants). In some cases, the patient has been identified as non-responsive to treatment with primary medications for the condition(s) targeted for treatment (e.g., non-responsive to antidepressants for the treatment of depression), and a renin inhibitor in combination with one or more neurogenic agents may be administered in a method for enhancing the responsiveness of the patient to a co-existing or preexisting treatment regimen.

In additional embodiments, the patient in need of neurogenic modulation suffers from premenstrual syndrome, post-partum depression, or pregnancy-related fatigue and/or depression, and the treatment comprises administering a therapeutically effective amount of a renin inhibitor in combination with one or more neurogenic agents. Without being bound by any particular theory, and offered to improve understanding of the disclosure, it is believed that levels of steroid hormones, such as estrogen, are increased during the menstrual cycle during and following pregnancy, and that such hormones can exert a modulatory effect on neurogenesis.

In some embodiments, the patient is a user of a recreational drug including but not limited to alcohol, amphetamines, PCP, cocaine, and opiates. Without being bound by any particular theory, and offered to improve understanding of the disclosure, it is believed that some drugs of abuse have a modulatory effect on neurogenesis, which is associated with depression, anxiety and other mood and affective disorders, as well as deficits in cognition, learning, and memory. Moreover, mood disorders are causative/risk factors for substance abuse, and substance abuse is a common behavioral symptom (e.g., self medicating) of mood disorders. Thus, substance abuse and mood disorders may reinforce each other, rendering patients suffering from both conditions non-responsive to treatment. Thus, in some embodiments, a renin inhibitor in combination with one or more neurogenic agents may be administered to treat patients suffering from substance abuse and/or mood disorders.

In further embodiments, the patient is on a co-existing and/or pre-existing treatment regimen involving administration of one or more prescription medications having a modulatory effect on neurogenesis. For example, the patient suffers from chronic pain and is prescribed one or more opiate/opioid medications; and/or suffers from ADD, ADHD, or a related disorder, and is prescribed a psychostimulant, such as ritalin, dexedrine, adderall, or a similar medication. Without being bound by any particular theory, and offered to improve understanding of the disclosure, it is believed that such medications may exert a modulatory effect on neurogenesis, leading to depression, anxiety and other mood disorders, as well as deficits in cognition, learning, and memory. Thus, a renin inhibitor in combination with one or more neurogenic agents may be administered to patients receiving these prescribed medications in order to treat the associated depression, anxiety, and/or other mood disorders, and/or to improve cognition.

In additional embodiments, the patient suffers from chronic fatigue syndrome; a sleep disorder; lack of exercise (e.g., elderly, infirm, or physically handicapped patients); and/or lack of environmental stimuli (e.g., social isolation); and the treatment comprises administering a therapeutically effective amount of a renin inhibitor in combination with one or more neurogenic agents.

In more embodiments, the patient is an individual having, or who is likely to develop, a disorder related to neural degeneration, neural damage and/or neural demyelination.

In yet additional embodiments, identifying a patient in need of neurogenesis modulation comprises selecting a population or sub-population of patients, or an individual patient, that is more amenable to treatment and/or less susceptible to side effects than other patients having the same disease or condition. In some embodiments, identifying a patient amenable to treatment with a renin inhibitor in combination with one or more neurogenic agents, comprises identifying a patient who has been exposed to a factor known to enhance neurogenesis, including but not limited to, exercise, hormones or other endogenous factors, and drugs taken as part of a pre-existing treatment regimen.

In some embodiments, a sub-population of patients is identified as being more amenable to neurogenesis modulation with a renin inhibitor in combination with one or more neurogenic agents, by taking a cell or tissue sample from prospective patients, isolating and culturing neural cells from the sample, and determining the effect of a renin inhibitor in combination with one or more neurogenic agents on the degree or nature of neurogenesis of the cells, thereby allowing selection of patients for which the therapeutic agent has a substantial effect on neurogenesis. Advantageously, the selection of a patient or population of patients in need of or amenable to treatment with a renin inhibitor in combination with one or more neurogenic agents, allows for more effective treatment of the disease or condition targeted for treatment than known methods using the same or similar compounds.

In some embodiments, the patient has suffered a CNS insult, such as a CNS lesion, a seizure (e.g., electroconvulsive seizure treatment; epileptic seizures), radiation therapy, chemotherapy and/or stroke or other ischemic injury. Without being bound by any particular theory, and offered to improve understanding of the disclosure, it is believed that some CNS insults/injuries leads to increased proliferation of neural stem cells, but that the resulting neural cells form aberrant connections which can lead to impaired CNS function and/or diseases, such as temporal lobe epilepsy. In other embodiments, a renin inhibitor in combination with one or more neurogenic agents may be administered to a patient who has suffered, or is at risk of suffering, a CNS insult or injury to stimulate neurogenesis.

Advantageously, stimulation of the differentiation of neural stem cells with a renin inhibitor in combination with one or more neurogenic agents, activates signaling pathways necessary for progenitor cells to effectively migrate and incorporate into existing neural networks or to block inappropriate proliferation.

Additionally, the disclosed methods provide for the application of a renin inhibitor in combination with one or more neurogenic agents to treat a subject or patient for a condition due to the anti-neurogenic effects of an opiate or opioid based analgesic.

In some embodiments, the administration of an opiate or opioid based analgesic, such as an opiate like morphine or other opioid receptor agonist, to a subject or patient results in a decrease in, or inhibition of, neurogenesis. The administration of a renin inhibitor in combination with one or more neurogenic agents would reduce the anti-neurogenic effect.

The disclosed embodiments include a method of treating post operative pain in a subject or patient by combining the administration of a renin inhibitor in combination with one or more neurogenic agents.

Other disclosed embodiments include methods for treating or preventing decreases in, or inhibition of, neurogenesis resulting from the use of an opioid receptor agonist. The methods comprise the administration of a therapeutically effective amount of a renin inhibitor in combination with one or more neurogenic agents. Non-limiting examples include cases involving an opioid receptor agonist, which decreases or inhibits neurogenesis, and drug addiction, drug rehabilitation, and/or prevention of relapse into addiction. In some embodiments, the opioid receptor agonist is morphine, opium or other opiate.

In additional embodiments, the disclosed methods may be used to treat subjects having, or diagnosed with, depression or other withdrawal symptoms from morphine or other agents which decrease or inhibit neurogenesis. This is distinct from the treatment of subjects having, or diagnosed with, depression independent of an opiate, such as that of a mental disorder, as disclosed herein. In further embodiments, the methods may be used to treat a subject with one or more chemical addiction or dependency, such as with morphine or other opiates, where the addiction or dependency is ameliorated or alleviated by an increase in neurogenesis.

In other embodiments, the methods described herein involve modulating neurogenesis in vitro or ex vivo with a renin inhibitor in combination with one or more neurogenic agents, such that a composition containing neural stem cells, neural progenitor cells, and/or differentiated neural cells can subsequently be administered to an individual to treat a disease or condition.

In some embodiments, the method of treatment comprises the steps of contacting a neural stem cell or progenitor cell with a renin inhibitor in combination with one or more neurogenic agents, to modulate neurogenesis, and transplanting the cells into a patient in need of treatment. Methods for transplanting stem and progenitor cells are known in the art, and are described, e.g., in U.S. Pat. Nos. 5,928,947; 5,817,773; and 5,800,539, and PCT Publication Nos. WO 01/176507 and WO 01/170243, all of which are incorporated herein by reference in their entirety. In some embodiments, methods described herein allow treatment of diseases or conditions by directly replenishing, replacing, and/or supplementing damaged or dysfunctional neurons. In further embodiments, methods described herein enhance the growth and/or survival of existing neural cells, and/or slow or reverse the loss of such cells in a neurodegenerative or other condition.

In alternative embodiments, the method of treatment comprises identifying, generating, and/or propagating neural cells in vitro or ex vivo in contact with a renin inhibitor in combination with one or more neurogenic agents, and transplanting the cells into a subject.

In another embodiment, the method of treatment comprises the steps of contacting a neural stem cell of progenitor cell with a renin inhibitor in combination with one or more neurogenic agents, to stimulate neurogenesis or neurodifferentiation, and transplanting the cells into a patient in need of treatment. Also disclosed are methods for preparing a population of neural stem cells suitable for transplantation, comprising culturing a population of neural stem cells (NSCs) in vitro, and contacting the cultured neural stem cells with a renin inhibitor in combination with one or more neurogenic agents. The disclosure further includes methods of treating the diseases, disorders, and conditions described herein by transplanting such treated cells into a subject or patient.

In additional embodiments, the disclosure includes a method of stimulating or increasing neurogenesis in a subject or patient with stimulation of angiogenesis in the subject or patient. The co-stimulation may be used to provide the differentiating and/or proliferating cells with increased access to the circulatory system. The neurogenesis is produced by administering a renin inhibitor in combination with one or more neurogenic agents.

As described herein, the disclosed embodiments include methods for treating diseases, disorders, and conditions of the central and/or peripheral nervous systems (CNS and PNS, respectively) by administering a renin inhibitor in combination with one or more neurogenic agents. The amount of a renin inhibitor in combination with one or more neurogenic agents may be any that results in a measurable relief of a disease condition like those described herein. As a non-limiting example, an improvement in the Hamilton depression scale (HAM-D) score for depression may be used to determine (such as quantitatively) or detect (such as qualitatively) a measurable level of improvement in the depression of a subject.

Non-limiting examples of symptoms that may be treated with the methods described herein include abnormal behavior, abnormal movement, hyperactivity, hallucinations, acute delusions, combativeness, hostility, negativism, withdrawal, seclusion, memory defects, sensory defects, cognitive defects, and tension. Non-limiting examples of abnormal behavior include irritability, poor impulse control, distractibility, and aggressiveness. Outcomes from treatment with the disclosed methods include improvements in cognitive function or capability in comparison to the absence of treatment.

Additional examples of diseases, disorders, and conditions of the central and/or peripheral nervous systems (CNS and PNS, respectively) and associated conditions treatable by the methods described herein include, but are not limited to, neurodegenerative diseases and disorders. Such diseases and disorders include but are not limited to Alzheimer's disease, Parkinson's disease, Huntington's disease (Huntington's Chorea), Lou Gehrig's disease, Pick's disease, epilepsy (seizures), multiple sclerosis, amyotrophic lateral sclerosis, progressive subcortical gliosis, progressive supranuclear palsy, thalmic degeneration syndrome, hereditary aphasia, Shy-Drager syndrome, Lewy body disease, cardiovascular diseases and conditions (i.e. infarcts, hemorrhage, cardiac disorders), mixed vascular, bacterial meningitis, Creutzfeld-Jacob Disease, and Cushing's disease, head injury, HIV disease and the conditions associated with such diseases and disorders such as but not limited to cognition, dementias (i.e. Parkinsonism dementia syndrome, senile dementia, memory disturbances/memory loss), delirium, amnestic disorders, depression and anxiety. In practice, a subject or patient may be afflicted with, or diagnosed with, one or more of the above mentioned diseases or disorders in any combination.

The disclosed embodiments also provide for the treatment of a nervous system disorder related to neural damage, cellular degeneration, mental disorders, cellular or tissue (neurological) trauma and/or injury (e.g., subdural hematoma or traumatic brain injury), toxic chemicals (e.g., heavy metals, alcohol, some medications), CNS hypoxia, or other neurologically related conditions. In practice, a therapeutically effective amount of a renin inhibitor in combination with one or more neurogenic agents may be administered to a subject or patient afflicted with, or diagnosed with, one or more central or peripheral nervous system disorders. Diagnosis may be performed by a skilled person in the applicable fields using known and routine methodologies which identify and/or distinguish these nervous system disorders from other conditions.

Non-limiting examples of nervous system disorders related to cellular degeneration include neurodegenerative disorders, neural stem cell disorders, neural progenitor cell disorders, and ischemic disorders. In some embodiments, an ischemic disorder comprises an insufficiency, or lack, of oxygen or angiogenesis, and non-limiting example include spinal ischemia, ischemic stroke, cerebral infarction, multi-infarct dementia. While these conditions may be present individually in a subject or patient, the disclosed methods also provide for the treatment of a subject or patient afflicted with, or diagnosed with, more than one of these conditions.

Non-limiting embodiments of nervous system disorders related to a mental disorder include affective disorders and schizophrenia. As used herein, an affective disorder refers to but is not limited to anxiety and depression. In practice, a subject or patient may be afflicted with, or diagnosed with, one or more of the above mentioned mental disorders in any combination.

Examples of nervous system disorders related to cellular or tissue trauma and/or injury include, but are not limited to neurological traumas and injuries, surgery related trauma and/or injury, retinal injury and trauma, injury related to epilepsy, spinal cord injury, brain injury, brain surgery, trauma related brain injury, trauma related to spinal cord injury, brain injury related to chemotherapy, spinal cord injury related to chemotherapy, brain injury related to radiation therapy, spinal cord injury related to radiation therapy, brain injury related to infection, spinal cord injury related to infection, brain injury related to inflammation, spinal cord injury related to inflammation, brain injury related to environmental toxin, and spinal cord injury related to environmental toxin and the conditions associated with such cellular or tissue trauma and/or injuries such as but not limited to cognition, dementias (i.e. dementia, memory disturbances and memory loss), delirium, amnestic disorders, mood disorders and anxiety disorders. In practice, a subject or patient may be afflicted with, or diagnosed with, one or more of the above mentioned cellular or tissue traumas and/or injuries in any combination.

Non-limiting examples of nervous system disorders related to other neurologically related conditions include learning disorders, memory disorders, age-associated memory impairment (AAMI) or age-related memory loss, autism, learning or attention deficit disorders (ADD or attention deficit hyperactivity disorder, ADHD), narcolepsy, sleep disorders and sleep deprivation (e.g., insomnia, chronic fatigue syndrome), cognitive disorders, epilepsy, injury related to epilepsy, and temporal lobe epilepsy and combinations thereof.

Other non-limiting examples of diseases and conditions treatable by the methods described herein include, but are not limited to, hormonal changes (e.g., depression and other mood disorders associated with puberty, pregnancy, or aging (e.g., menopause)); and lack of exercise (e.g., depression or other mental disorders in elderly, paralyzed, or physically handicapped patients); infections (e.g., HIV); genetic abnormalities (down syndrome); metabolic abnormalities (e.g., vitamin B12 or folate deficiency); hydrocephalus; memory loss separate from dementia, including mild cognitive impairment (MCI), age-related cognitive decline, and memory loss resulting from the use of general anesthetics, chemotherapy, radiation treatment, post-surgical trauma, or therapeutic intervention; and diseases of the of the peripheral nervous system (PNS), including but not limited to, PNS neuropathies (e.g., vascular neuropathies, diabetic neuropathies, amyloid neuropathies, and the like), neuralgias, neoplasms, myelin-related diseases, and the like.

Other conditions that may be beneficially treated by increasing neurogenesis are known in the art (see e.g., U.S. Publication Nos. 20020106731, 2005/0009742 and 2005/0009847, 20050032702, 2005/0031538, 2005/0004046, 2004/0254152, 2004/0229291, and 2004/0185429, herein incorporated by reference in their entirety).

Renin, also known as angiotensinogenase, is a circulating enzyme that participates in the renin-angiotensin system that mediates extracellular volume, arterial vasoconstriction, and consequently mean arterial blood pressure. The enzyme is secreted by the kidneys from specialized juxtaglomerular cells in response to decreases in glomerular filtration rate (a consequence of low blood volume), diminished filtered sodium chloride and sympathetic nervous system innervation. The enzyme circulates in the blood stream and hydrolyzes angiotensinogen secreted from the liver into the peptide angiotensin I. Angiotensin I is further cleaved in the lungs by endothelial bound angiotensin converting enzyme (ACE) into angiotensin II, the final active peptide. The normal concentration in adult human plasma is 1.98-24.6 ng/L in the upright position.

The primary structure of renin precursor consists of 406 amino acids with a pre and a pro segment carrying 20 and 46 amino acids respectively. Mature renin contains 340 amino acids and has a mass of 37 kD.

Renin activates the renin-angiotensin system by cleaving angiotensinogen, produced by the liver, to yield angiotensin I, which is further converted into angiotensin II by ACE, the angiotensin-converting enzyme primarily within the capillaries of the lungs. Angiotensin II then constricts blood vessels, increases the secretion of ADH and alsosterone, and stimulates the hypothalamus to activate the thirst reflex, each leading to an increase in blood pressure. Renin is secreted from juxtaglomerular cells (of the afferent arterioles), which are activated via signaling (the release of prostaglandins) from the macula densa, which respond to the rate of fluid flow through the distal tubule, by decreases in renal perfusion pressure (through stretch receptors in the vascular wall), and by nervous stimulation, mainly through beta-1 receptor activation. A drop in the rate of flow past the macula densa implies a drop in renal filtration pressure. Renin's primary function is therefore to eventually cause an increase in blood pressure, leading to restoration of perfusion pressure in the kidneys.

The gene for renin, REN, spans 12 kb of DNA and contains 8 introns. It produces several mRNA that encode different REN isoforms.

Human Renin is secreted by at least 2 cellular pathways: a constitutive pathway for the secretion of prorenin and a regulated pathway for the secretion of mature renin.

Plasma renin activity (PRA) is a measure of renin and is used in various diagnoses from hypertension to renin secreting tumors. An over-active renin-angiotension system leads to vasoconstriction and retention of sodium and water. These effects lead to hypertension. Therefore, renin inhibitors can be used for the treatment of hypertension.

Renin inhibitors, or inhibitors of renin, are a new group of pharmaceuticals that are used primarily in treatment of hypertension. They act on the juxtaglomerular cells of the kidney, which produces renin in response to decreased blood. Examples of renin inhibitors include but are not limited to Aliskiren and Remikiren.

Aliskiren ((2S,4S,5S,7S)-5-amino-N-(2-carbamoyl-2-methyl-propyl)-4-hydroxy-7-{[4-methoxy-3-(3-methoxypropoxy)phenyl]methyl}-8-methyl-2-propan-2-yl-nonanamide), is a first-in-class oral renin inhibitor and has the following structure:

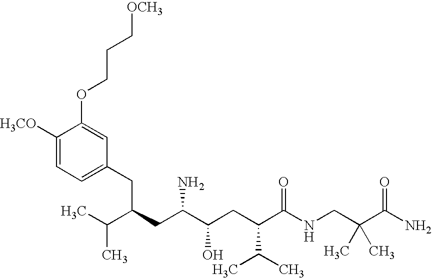

Aliskerin was developed by Novartis in conjunction with the biotech company Speedel. It was approved by the US Food and Drug Administration in 2007 for the treatment of hypertension. The trade name for aliskiren is Tekturna in the United States, and Rasilez in the United Kingdom. It is an octanamide, the first known representative of a new class of completely non-peptide, low-molecular weight, orally active transition-state renin inhibitors. Designed through the use of molecular modeling techniques, it is a potent and specific in vitro inhibitor of human renin (IC50 in the low nanomolar range), with a plasma half-life of ≈24 hours. Tekturna has good water solubility and low lipophilicity and is resistant to biodegradation by peptidases in the intestine, blood circulation, and the liver.

Remikiren ((2R)-2-(tert-butylsulfonylmethyl)-N-[(2S)-1-{[(2R,3S,4R)-1-cyclohexyl-4-cyclopropyl-3,4-dihydroxybutan-2-yl]amino}-3-(3H-imidazol-4-yl)-1-oxopropan-2-yl]-3-phenylpropanamide) is a renin inhibitor under development for the treatment of hypertension (high blood pressure) by Hoffmann-La Roche (1996) and has the following structure:

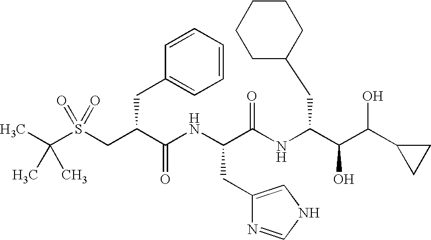

The disclosure further provides for analogs of Aliskiren as described below derived from structural Formula (I):

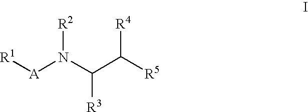

or a salt, hydrate, solvate or N-oxide thereof, wherein:

A represents the bivalent residue of a natural or unnatural amino acid wherein the N terminus is bound to R¹ and the C terminus is bound to the NR²— group;

R¹ is independently selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, OR⁶, S(O)_{b}R⁶, NR⁶R⁷, CONR⁶R⁷, CO₂R⁶, NR⁶CO₂R⁷, NR⁶CONR⁷R⁸, NR⁶CSNR⁷R⁸, NR⁶C(═NH)NR⁷R⁸, SO₂NR⁶R⁷, NR⁶SO₂R⁷, NR⁶SO₂NR⁷R⁸, P(O)(OR⁶)(OR⁷), and P(O)(R⁶)(OR⁷);

b=0, 1, or 2;

R⁶-R⁸ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl and substituted heteroarylalkyl or alternatively, R⁶ and R⁷, R⁶ and R⁸, or R⁷ and R⁸, together with the atoms to which they are bonded form a cycloheteroalkyl or substituted cycloheteroalkyl ring;

R² is selected from the group consisting of hydrogen, alkyl substituted alkyl

R³ is selected from the group consisting of hydrogen, alkyl, substituted alkyl; C₁-C₆ alkylcycloalkyl, or C₁-C₆ alkylsubstituted cycloalkyl;

R⁴ is selected from hydroxy, alkoxyl, aryloxy or heteroaryloxy, alkoxycarbonyl, substituted alkoxycarbonyl, carbamoyl and substituted carbamoyl;

R⁵ is selected from the group consisting of alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, and substituted heteroarylalkyl;

In some embodiments disclosed herein, R¹ forms a group having the formula (a):

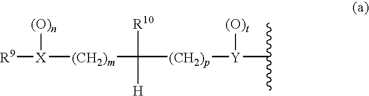

wherein:

R⁹ is selected from the group consisting of alkyl, substituted alkyl, heteroalkyl, substituted heteroalkyl, heteroaryl, substituted heteroaryl, arylalkyl, substituted arylalkyl, heteroarylalkyl, substituted heteroarylalkyl, NR⁶R⁷, NR⁶CO₂R⁷, NR⁶CONR⁷R⁸, NR⁶CSNR⁷R⁸, NR⁶C(═NH)NR⁷R⁸, NR⁶SO₂R⁷, and NR⁶SO₂NR⁷R⁸;

R⁶-R⁸ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl and substituted heteroarylalkyl or alternatively, R⁶ and R⁷, R⁶ and R⁸ or R⁷ and R⁸, together with the atoms to which they are bonded form a cycloheteroalkyl or substituted cycloheteroalkyl ring;

X is N, C, O, S or P;

n=0, 1, or 2;

m=0, 1, or 2;

R¹⁰ is selected from the group consisting of hydrogen, alkyl, substituted alkyl, heteroalkyl, substituted heteroalkyl, heteroaryl, substituted heteroaryl, heteroarylalkyl, or substituted heteroarylalkyl;

p=0, or 1;

Y is selected from C, or S; and

t=0, 1, or 2.

In some embodiments disclosed herein, R⁵ forms a group having the formula (b) below:

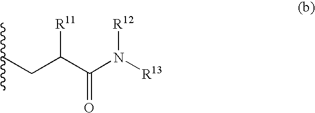

wherein:

R¹¹ is selected from the group consisting of alkyl, or substituted alkyl, and R¹² and R¹³ are independently selected from hydrogen, C₁-C₁₀ alkyl, and C₁-C₁₀ substituted alkyl, C₃-C₈ cycloalkyl, C₃-C₈ cycloalkyl-C₁-C₆ alkyl.

Another embodiment disclosed herein provides compounds having structural Formula (II):

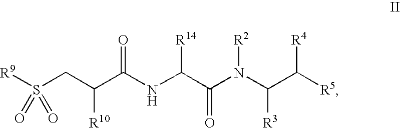

wherein:

R⁹ is selected from the group consisting of alkyl, substituted alkyl, heteroalkyl, substituted heteroalkyl, heteroaryl, substituted heteroaryl, arylalkyl, substituted arylalkyl, heteroarylalkyl, substituted heteroarylalkyl, NR⁶R⁷, NR⁶CO₂R⁷, NR⁶CONR⁷R⁸, NR⁶CSNR⁷R⁸, NR⁶C(═NH)NR⁷R⁸, NR⁶SO₂R⁷, and NR⁶SO₂NR⁷R⁸;

R⁶-R⁸ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl and substituted heteroarylalkyl or alternatively, R⁶ and R⁷, R⁶ and R⁸ or R⁷ and R⁸, together with the atoms to which they are bonded form a cycloheteroalkyl or substituted cycloheteroalkyl ring;

R¹⁰ is a substituted alkyl group as shown: —(CH₂)ᵥ—Ar¹, wherein Ar¹ is a substituted or unsubstituted five or six membered aryl, or heteroaryl ring and v=1 or 2;

R¹⁴ is selected from methyl, cyclohexylmethyl, hydroxymethyl, phenylmethyl, substituted phenylmethyl, imidazolylmethyl, and thioimidazolylmethyl;

R² is selected from the group consisting of hydrogen, C₁-C₆ alkyl, C₁-C₆ substituted alkyl;

R³ is selected from the group consisting of hydrogen, alkyl, substituted alkyl, C₁-C₆ alkylcycloalkyl, or C₁-C₆ alkylsubstituted cycloalkyl;

R⁴ is hydroxy, or alkoxyl. In some instances R⁴ may be alkoxyl aryloxy or heteroaryloxy, alkoxycarbonyl, substituted alkoxycarbonyl, carbamoyl and substituted carbamoyl or a hydroxyl that has been otherwise modified by an organic radical that can be removed under physiological conditions such that the cleavage products are physiologically tolerable at the resulting concentrations;

R⁵ is selected from the group consisting of C₁-C₈ alkyl, substituted C₁-C₈ alkyl, or R⁵, forms a group having the formula (b) above wherein R¹² and R¹³ are independently selected from hydrogen, C₁-C₈ alkyl, and C₁-C₈ substituted alkyl, C₃-C₈ cycloalkyl, C₃-C₈ cycloalkyl-C₁-C₆ alkyl.

In another embodiment, the disclosure provides compounds having structural Formula (III):

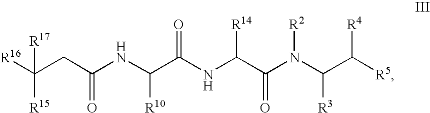

wherein:

R¹⁵, R¹⁶ and R¹⁷ are independently selected from the group consisting of hydrogen, amino, C₁-C₆ alkylamino and C₁-C₆ alkyl, or alternatively, R¹⁵ and R¹⁶ together with the atoms to which they are bonded form a cycloalkyl, substituted cycloalkyl or heteroalkyl ring and R¹⁷ is an amino group;

R¹⁰ is a substituted alky group as shown in the formula: —(CH₂)ᵥ—Ar¹, wherein Ar¹ is a substituted or unsubstituted five or six membered aryl, or heteroaryl ring and v=1 or 2;

R¹⁴ is selected from methyl, cyclohexylmethyl, hydroxymethyl, phenylmethyl, substituted phenylmethyl, imidazolylmethyl, and thioimidazolylmethyl;

R² is selected from the group consisting of hydrogen, C₁-C₆ alkyl, C₁-C₆ substituted alkyl;

R³ is selected from the group consisting of hydrogen, alkyl, substituted alkyl, C₁-C₆ alkylcycloalkyl, or C₁-C₆ alkylsubstituted cycloalkyl;

R⁴ is hydroxy, or alkoxyl. In some instances R⁴ may be alkoxyl aryloxy or heteroaryloxy, alkoxycarbonyl, substituted alkoxycarbonyl, carbamoyl and substituted carbamoyl or a hydroxyl that has been otherwise modified by an organic radical that can be removed under physiological conditions such that the cleavage products are physiologically tolerable at the resulting concentrations;

R⁵ is selected from the group consisting of C₁-C₈ alkyl, substituted C₁-C₈ alkyl.

In another embodiment, the disclosure provides compounds having structural formula IV:

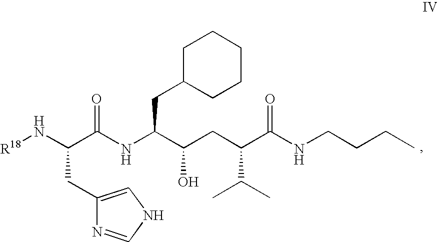

wherein:

R¹⁸ is selected from alkyl, substituted alkyl, heteroalkyl, substituted heteroalkyl, heteroaryl, substituted heteroaryl, arylalkyl, substituted arylalkyl, heteroarylalkyl, substituted heteroarylalkyl, alkenyl, alkynyl, alkoxy, aryloxy, heteroaryloxy, arylalkyl, heteroarylalkyl, arylalkoxy, heteroarylalkoxy, amino, alkyl- and dialkylamino groups, carbamoyl groups, alkylcarbonyl, alkoxycarbonyl, alkylaminocarbonyl, dialkylamino carbonyl, arylcarbonyl, aryloxycarbonyl, alkylsulfonyl, arylsulfonyl, cycloalkyl, acyl and substituted acyl groups, phosphate or phosphonyl groups, sulfamyl groups, sulfonyl groups, sulfinyl groups, and combinations thereof.

In another embodiment, the disclosure provides compounds having structural formula IV, wherein:

R¹⁸ is selected from indolyl-2-carbonyl, cyclohepta[b]-pyrrolyl-5-carbonyl, 2(S)-pivaloyloxy-3-phenyl-propionyl, 2(R,S)-dimethoxyphosphoryl-3-phenyl-propionyl, 2(S)dimethoxyphosphoryl-3-phenyl-propionyl, 2(R)-dimethoxyphosphoryl-3-phenyl-propionyl, 2(R,S)-benzyl-5,5-dimethyl-4-oxo-hexanoyl, 2(S)-benzyl-5,5-dimethyl-4-oxo-hexanoyl, 2(R)-benzyl-5,5-dimethyl-4-oxo-hexanoyl, 2(R,S)-benzyl-4,4-dimethyl-3-oxo-pentanoyl, 2(R,S)ethoxycarbonyl-3-alpha-naphthyl-propionyl, or 2(S)-pivaloyl-3-phenylpropionyl;

In another embodiment, the disclosure provides compounds having the structures below:

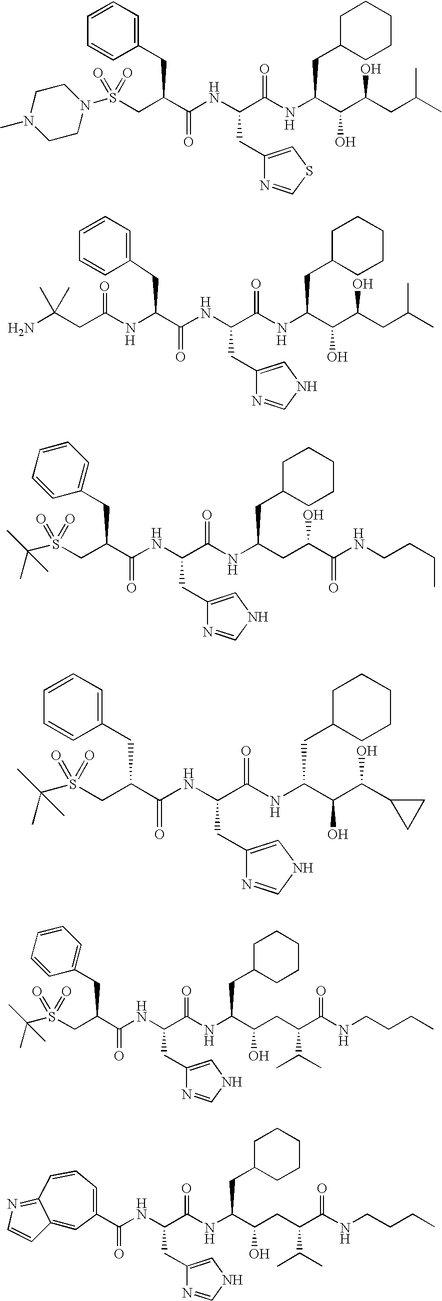

or a salt, hydrate, solvate or N-oxide thereof.

In another aspect, the disclosure provides compounds of structural Formula (V):

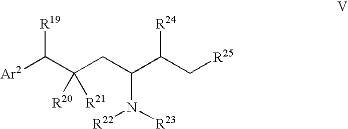

or a salt, hydrate, solvate or N-oxide thereof wherein:

R¹⁹ is selected from hydrogen, hydroxy, or alkoxyl. In some instances R¹⁹ may be alkoxyl aryloxy or heteroaryloxy, alkoxycarbonyl, substituted alkoxycarbonyl, carbamoyl and substituted carbamoyl or a hydroxyl that has been otherwise modified by an organic radical that can be removed under physiological conditions such that the cleavage products are physiologically tolerable at the resulting concentrations;

R²⁰ and R²¹ are independently selected from the group consisting of hydrogen, C₁-C₈ alkyl, C₁-C₈ substituted alkyl, C₁-C₆ alkylcycloalkyl, or C₁-C₆ alkyl-substituted-cycloalkyl, heteroalkyl, substituted heteroalkyl, or alternatively, R²⁰ and R²¹ together with the atoms to which they are bonded form a cycloalkyl, substituted cycloalkyl, cycloalkene, substituted cycloalkene, cycloheteroalkyl or substituted cycloheteroalkyl ring;

R²² and R²³ are independently selected from the group consisting of C₁-C₈ alkyl, C₁-C₈ substituted alkyl, C₁-C₈ alkoxy, C₁-C₈ substituted alkoxy, C₁-C₈ alkylamino, C₁-C₈ substituted alkylamino, aryl, substituted aryl, arylalkyl, substituted arylalkyl, acyl, substituted acyl, heteroalkyl, substituted heteroalkyl, heteroaryl, substituted heteroaryl, heteroarylalkyl, and substituted heteroarylalkyl or alternatively, R²² and R²³, R²² and R²⁴ or R²³ and R²⁴, together with the atoms to which they are bonded form a cycloheteroalkyl or substituted cycloheteroalkyl ring;

R²⁴ is selected from hydrogen, hydroxy, or alkoxyl. In some instances R²⁴ may be alkoxyl aryloxy or heteroaryloxy, alkoxycarbonyl, substituted alkoxycarbonyl, carbamoyl and substituted carbamoyl or a hydroxyl that has been otherwise modified by an organic radical that can be removed under physiological conditions such that the cleavage products are physiologically tolerable at the resulting concentrations or alternatively, R²⁴ together with R²³, or R²⁴ together with R²², with the atoms to which they are bonded form a cycloheteroalkyl or substituted cycloheteroalkyl ring;

Ar² is a substituted or unsubstituted five or six membered aryl, or heteroaryl ring; and

R²⁵ is selected from the group consisting of alkyl, substituted alkyl, aryl, substituted aryl, arylalkyl, substituted arylalkyl, alkylcarbonyl, substituted alkylcarbonyl, heteroalkyl, substituted heteroalkyl, heteroaryl, substituted heteroaryl, heteroarylalkyl, and substituted heteroarylalkyl.

In another embodiment, the disclosure provides compounds having structural Formula (VI):

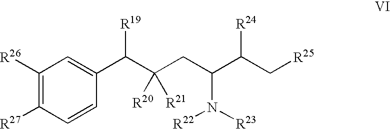

R¹⁹ is selected from hydrogen, hydroxy, or alkoxyl. In some instances R¹⁹ may be alkoxyl aryloxy or heteroaryloxy, alkoxycarbonyl, substituted alkoxycarbonyl, carbamoyl and substituted carbamoyl or a hydroxyl that has been otherwise modified by an organic radical that can be removed under physiological conditions such that the cleavage products are physiologically tolerable at the resulting concentrations;

R²⁰ and R²¹ are independently selected from the group consisting of hydrogen, C₁-C₈ alkyl, C₁-C₈ substituted alkyl, C₁-C₆ alkylcycloalkyl, or C₁-C₆ alkyl-substituted-cycloalkyl, heteroalkyl, substituted heteroalkyl, or alternatively, R²⁰ and R²¹ together with the atoms to which they are bonded form a cycloalkyl, substituted cycloalkyl, cycloalkene, substituted cycloalkene, cycloheteroalkyl or substituted cycloheteroalkyl ring;

R²² and R²³ are independently selected from the group consisting of hydrogen, C₁-C₈ alkyl, C₁-C₈ substituted alkyl, C₁-C₈ alkoxy, C₁-C₈ substituted alkoxy, C₁-C₈ alkylamino, C₁-C₈ substituted alkylamino, aryl, substituted aryl, arylalkyl, substituted arylalkyl, acyl, substituted acyl, heteroalkyl, substituted heteroalkyl, heteroaryl, substituted heteroaryl, heteroarylalkyl, and substituted heteroarylalkyl or alternatively, R²² and R²³, R²² and R²⁴ or R²³ and R²⁴, together with the atoms to which they are bonded form a cycloheteroalkyl or substituted cycloheteroalkyl ring;

R²⁴ is selected from hydrogen, hydroxy, or alkoxyl. In some instance R²⁴ may be alkoxyl aryloxy or heteroaryloxy, alkoxycarbonyl, substituted alkoxycarbonyl, carbamoyl and substituted carbamoyl or a hydroxyl that has been otherwise modified by an organic radical that can be removed under physiological conditions such that the cleavage products are physiologically tolerable at the resulting concentrations or alternatively, R²⁴ together with R²³, or R²⁴ together with R²², with the atoms to which they are bonded form a cycloheteroalkyl or substituted cycloheteroalkyl ring;

R²⁶ and R²⁷ are independently selected from the group consisting of C₁-C₆ alkyl, C₁-C₆ substituted alkyl, C₁-C₆ alkoxy, C₁-C₆ alkoxyalkyl, C₁-C₆ alkoxy-C₁-C₄ alkyloxy;

R²⁵ is selected from the group consisting of alkyl, substituted alkyl, aryl, substituted aryl, arylalkyl, substituted arylalkyl, alkylcarbonyl, substituted alkylcarbonyl, heteroalkyl, substituted heteroalkyl, heteroaryl, substituted heteroaryl, heteroarylalkyl, and substituted heteroarylalkyl.

In another embodiment disclosed herein, R²⁵, forms a group having the formula (c):

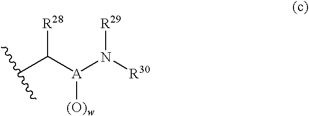

wherein:

R²⁸ is C₁-C₆ alkyl;

A is selected from C or S;

w is 0, 1, or 2, and

R²⁹ and R³⁰ are independently selected from hydrogen, C₁-C₆ alkyl, C₁-C₆ substituted alkyl, C₁-C₆ alkoxy, C₁-C₆ alkoxyalkyl, C₁-C₆ alkoxy-C₁-C₄ alkyloxy, NR⁶CO₂R⁷, or NR⁶CONR⁷R⁸ with R⁶, R⁷ and R⁸ as described above.

In another embodiment disclosed herein, the disclosure provides compounds having structural Formula (VII):

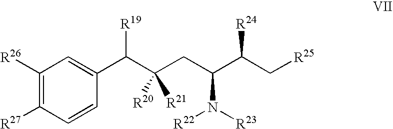

wherein:

R¹⁹ and R²⁰ are hydrogen;

R²¹ is selected from the group consisting of hydrogen, C₁-C₈ alkyl, C₁-C₈ substituted alkyl, C₁-C₆ alkylcycloalkyl;

R²² and R²³ are independently selected from the group consisting of Hydrogen C₁-C₈ alkyl, C₁-C₈ substituted alkyl, C₁-C₈ alkoxy, C₁-C₈ substituted alkoxy, C₁-C₈ alkylamino, C₁-C₈ substituted alkylamino, acyl, substituted acyl, or alternatively, R²² and R²³, R²² and R²⁴ or R²³ and R²⁴, together with the atoms to which they are bonded form a cycloheteroalkyl or substituted cycloheteroalkyl ring;

R²⁴ is selected from hydrogen, hydroxy, or alkoxyl. In some instances R²⁴ may be alkoxyl aryloxy or heteroaryloxy, alkoxycarbonyl, substituted alkoxycarbonyl, carbamoyl and substituted carbamoyl or a hydroxyl that has been otherwise modified by an organic radical that can be removed under physiological conditions such that the cleavage products are physiologically tolerable at the resulting concentrations or alternatively, R²⁴ together with R²³, or R²⁴ together with R²², with the atoms to which they are bonded form a cycloheteroalkyl or substituted cycloheteroalkyl ring;

R²⁵ forms a group having the formula (c) below:

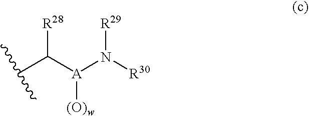

wherein:

R²⁸ is C₁-C₆ alkyl;

A is C;

w is 1;

R²⁹ and R³⁰ are independently selected from hydrogen, C₁-C₆ alkyl, C₁-C₆ substituted alkyl, C₁-C₆ alkoxy, C₁-C₆ alkoxyalkyl, C₁-C₆ alkoxy-C₁-C₄ alkyloxy, NR⁶CO₂R⁷, or NR⁶CONR⁷R⁸ with R⁶, R⁷ and R⁸ as described above; and

R²⁶ is selected from the group consisting of C₁-C₆ alkoxy, C₁-C₆ alkoxyalkyl, C₁-C₆ alkoxy-C₁-C₄ alkyloxy; and R²⁷ is C₁-C₄ alkoxy.

In another embodiment, the disclosure provides compounds having structural Formula (VIII):

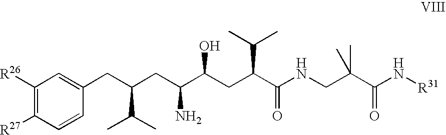

wherein:

R²⁶ is selected from the group consisting of methoxy-C₂-C₄ alkoxy, and R²⁷ is methoxy or ethoxy.

R³¹ is Hydrogen or C₁-C₆ alkyl.

In another embodiment, the disclosure provides compounds having the structure:

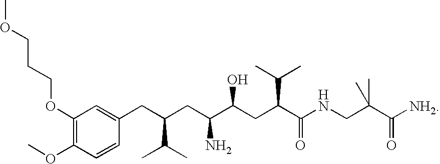

In another embodiment, the disclosure provides compounds having structural Formula (IX):

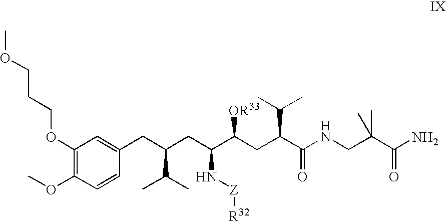

wherein:

Z represents the group —C(═X) where X is O, NH, or S; or SO₂; and R³² is selected from the group consisting of C₁-C₈ alkyl, C₁-C₈ substituted alkyl, C₁-C₈ alkoxy, C₁-C₈ substituted alkoxy, C₁-C₈ alkylamino, C₁-C₈ substituted alkylamino, aryl and substituted aryl and R³³ is hydrogen.

In another embodiment, the disclosure provides compounds wherein R³² and R³³ together form a single bond or a methylene; and Z represents the group —C(═X) where X represents NH, S or O.

In another embodiment, the disclosure provides compounds having any of the structures:

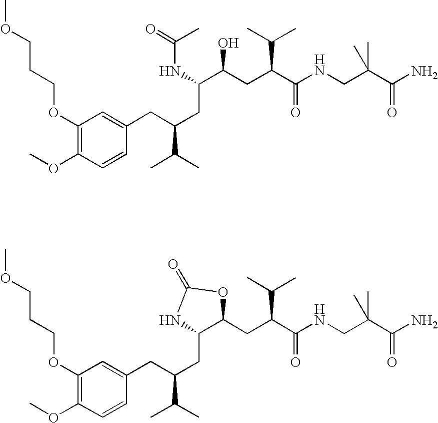

In another embodiment, the disclosure provides compounds having structural Formula (X):

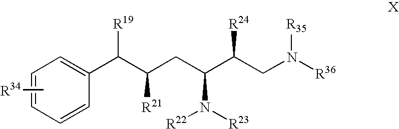

wherein:

R¹⁹ is selected from hydrogen, hydroxy, or alkoxyl. In some instances R¹⁹ may be alkoxyl aryloxy or heteroaryloxy, alkoxycarbonyl, substituted alkoxycarbonyl, carbamoyl and substituted carbamoyl or a hydroxyl that has been otherwise modified by an organic radical that can be removed under physiological conditions such that the cleavage products are physiologically tolerable at the resulting concentrations;

R²¹ is selected from the group consisting of hydrogen, C₁-C₈ alkyl,

R²² and R²³ are independently selected from the group consisting of hydrogen, C₁-C₄ alkyl, C₁-C₄ substituted alkyl, C₁-C₈ alkoxycarbonyl, C₁-C₈ substituted alkoxycarbonyl, C₁-C₈ acyl, substituted C₁-C₈ acyl;

R²⁴ is selected from hydrogen, hydroxy, or alkoxyl. In some instances R²⁴ may be alkoxyl aryloxy or heteroaryloxy, alkoxycarbonyl, substituted alkoxycarbonyl, carbamoyl and substituted carbamoyl or a hydroxyl that has been otherwise modified by an organic radical that can be removed under physiological conditions such that the cleavage products are physiologically tolerable at the resulting concentrations.

R³⁴ may be from 1 to 4 radicals, which in each case is selected independently from the group consisting of hydrogen, halogen, perfluoroalkyl, perfluoroalkoxy, alkyl, substituted alkyl, alkenyl, substituted alkenyl, hydroxy, aryl, substituted aryl, arylalkyl, substituted arylalkyl, alkylcarbonyl, substituted alkylcarbonyl, heteroalkyl, substituted heteroalkyl, heteroaryl, substituted heteroaryl, heteroarylalkyl, and substituted heteroarylalkyl; oxo, mercapto, alkylthio, alkoxy, aryloxy, heteroaryloxy, arylalkyl, heteroarylalkyl, arylalkoxy, heteroarylalkoxy, amino, alkyl- and dialkylamino, carbamoyl, alkylcarbonyl, carboxyl, alkoxycarbonyl, alkylaminocarbonyl, dialkylamino carbonyl, arylcarbonyl, aryloxycarbonyl, alkylsulfonyl, arylsulfonyl, cycloalkyl, cyano, C₁-C₆ alkylthio, arylthio, nitro, keto, acyl, phosphate or phosphonyl, sulfamyl, sulfonyl and, sulfinyl.

R³⁵ and R³⁶ are independently selected from the group consisting of hydrogen, cyano, hydroxyl, C₁-C₈ alkyl, C₁-C₈ substituted alkyl, C₃-C₈ cycloalkyl, C₃-C₈ substituted cycloalkyl, C₁-C₈ acyl, substituted C₁-C₈ acyl; In some instances, R³⁵ and R³⁶ together with the nitrogen atom to which they are bound form a 4 to 8 member heterocyclic ring or a substituted 4 to 8 member heterocyclic ring.

As a preferred embodiment, the compound having structural Formula (X):

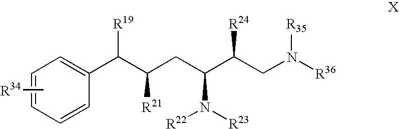

wherein:

R¹⁹ is hydrogen;

R²¹ is C₁-C₈ alkyl;

R²² and R²³ are both hydrogen;

R²⁴ is hydroxyl;

R³⁴ may be from 1 to 4 radicals, which in each case is selected independently from the group consisting of hydrogen, halogen, C₁-C₈ alkyl, C₁-C₈ substituted alkyl, trifluoromethyl, C₁-C₄ alkoxy-C₁-C₄ alkyl, C₁-C₄ alkoxy-C₁-C₄ alkoxy-C₁-C₄ alkyl, C₁-C₈ alkoxy, C₁-C₄ alkoxy-C₁-C₄ alkoxy;

R³⁵ and R³⁶ are independently selected from the group consisting of hydrogen, cyano, hydroxyl, C₁-C₈ alkyl, C₁-C₈ substituted alky, C₃-C₈ cycloalkyl, C₃-C₈ substituted cycloalkyl, C₁-C₈ acyl, substituted C₁-C₈ acyl; In some instances, R³⁵ and R³⁶ together with the nitrogen atom to which they are bound form a 4 to 8 member heterocyclic ring or a substituted 4 to 8 member heterocyclic ring.

In another embodiment, the disclosure provides compounds having structural Formula (XI):

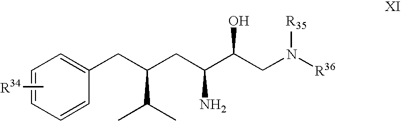

wherein:

R³⁴ may be from 1 to 4 radicals, which in each case is selected independently from the group consisting of hydrogen, halogen, C₁-C₈ alkyl, C₁-C₈ substituted alkyl, trifluoromethyl, C₁-C₄ alkoxy-C₁-C₄ alkyl, C₁-C₄ alkoxy-C₁-C₄ alkoxy-C₁-C₄ alkyl, C₁-C₈ alkoxy, C₁-C₄ alkoxy-C₁-C₄ alkoxy;

R³⁵ and R³⁶ together with the nitrogen atom to which they are bound form a heterocyclic ring or a substituted heterocyclic ring selected from pyrrolidinyl, piperidinyl, pyridinyl, piperazinyl, morpholino, thiomorpholino, furanyl, tetrahydrofuranyl, pyranyl tetrahydropyranyl, thiazolyl, oxazolyl, imidazolyl, indolinyl, isoindolinyl, 2,3-dihydrobenzimidazolyl, 1,2,3,4-tetrahydroisoquinolinyl, 1,2,3,4-tetrahydro-1,3-benzodiazinyl, 1,2,3,4-tetrahydro-1,4-benzodiazinyl, 3,4-dihydro-2H-1,4-benzoxazinyl, 3,4-dihydro-2H-1,4-benzothiazinyl, 3,4,5,6,7,8-hexahydro-2H-1,4-benzoxazinyl, 3,4,5,6,7,8-hexahydro-2H-1,4-benzothiazinyl, 9-azabicyclo[3.3.1]non-9-yl, 1-azepan-1-yl, 2,8-diazaspiro[4.5]dec-8-yl, octahydroisoindol-2-yl, 4-azatricyclo[5.2.1.0^{2,6}]dec-4-yl, 3-azabicyclo[3.2.1]oct-3-yl, 3,7-diazabicyclo[3.3.1]non-3-yl, 3-azabicyclo[3.3.1]non-3-yl, 3-azabicyclo[3.2.1]oct-8-yl, 3-azabicyclo[3.2.2]non-3-yl, 2,3,4,5-tetrahydro-1H-1-benzo[6,7b]azepinyl and 5,6-dihydrophenanthridinyl.

The 5-HT₃ antagonists are a class of medications that act as receptor antagonists at the 5-hydroxytryptamine-3-receptor (5-HT₃ receptor), a subtype of serotonin receptor found in the terminals of the vagus nerve and in certain areas of the brain. With the notable exception of alosetron and cilansetron, which are used in the treatment of irritable bowel syndrome, all 5-HT₃ antagonists are antiemetics, used in the prevention and treatment of nausea and vomiting. They are particularly effective in controlling the nausea and vomiting produced by cancer chemotherapy, especially that caused by highly emetogenic drugs such as cisplatin. When used for this purpose, they may be given alone or, more frequently, with a glucocorticoid, usually dexamethasone. They are usually given intravenously, shortly before administration of the chemotherapeutic agent, although some authors have argued that oral administration may be preferred. The concomitant administration of a NK1 receptor antagonist such as aprepitant, significantly increases the efficacy of 5-HT₃ antagonists in preventing both acute and delayed CINV. The 5-HT₃ antagonists may be identified by the suffix -setron, and are classified under code A04AA of the WHO's Anatomical Therapeutic Chemical Classification System.

The 5-HT₃ antagonists are also indicated in the prevention and treatment of radiation-induced nausea and vomiting (RINV), when needed, and postoperative nausea and vomiting (PONV). Although they are more effective at controlling CINV—where they stop symptoms altogether in up to 70% of people, and reduce them in the remaining 30%—they are just as effective as other agents for PONV. 5-HT₃ antagonists are ineffective in controlling motion sickness.

Ondansetron may be useful in treating antipsychotic-induced tardive dyskinesia in people with schizophrenia when used alone, or as an adjunct to haloperidol, wherein people taking both drugs experienced fewer of the adverse effects commonly associated with haloperidol.

Azasetron (N-(1-azabicyclo(2.2.2)oct-3-yl)-6-chloro-4-methyl-3-oxo-3,4-dihydro-2H-1,4-benzoxazine-8-carboxamide hydrochloride) is a potent and selective 5-HT3 receptor antagonist that inhibits the chemotherapy induced nausea and vomiting in animals and humans. This compound has the following structure:

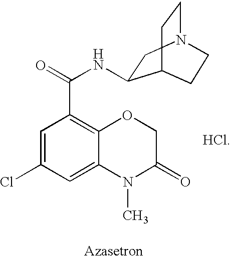

Ondansetron (9-methyl-3-[(2-methyl-1H-imidazol-1-yl)methyl]-1,2,3,9-tetrahydro-carbazol-4-one) is a serotonin 5-HT₃ receptor antagonist

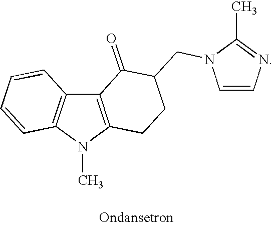

This compound is used mainly as an antiemetic to treat nausea and vomiting following chemotherapy. Its effects are thought to be on both peripheral and central nerves. One part is to reduce the activity of the vagus nerve, which is a nerve that activates the vomiting center in the medulla oblongata, the other is a blockage of serotonin receptors in the chemoreceptor trigger zone. It does not have much effect on vomiting that is due to motion sickness. This drug does not have any effect on dopamine receptors or muscarinic receptors.

Ondansetron is currently marketed by GlaxoSmithKline (GSK) under the trade name Zofran. The drug is administered 1-3 times daily, depending on the severity of nausea and/or vomiting. The normal oral dose for adults and children over the age of 12, is 8 mg initially, followed by a second dose of 8 mg, eight hours later. The drug is then administered once every 12 hours, usually not for more than 2-3 days. Following oral administration, it takes about 1.5-2 hours to reach maximum plasma concentrations. This drug is removed from the body by the liver and kidneys.

Tropisetron ([(1S,5S)-8-methyl-8-azabicyclo[3.2.1]oct-3-yl]1H-indole-3-carboxylate) is a serotonin 5-HT₃ receptor antagonist and has the following structure:

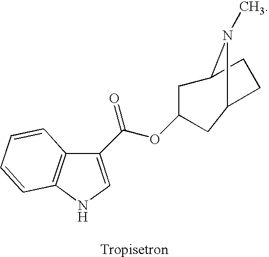

This compound is used mainly as an antiemetic to treat nausea and vomiting following chemotherapy. This drug has also been used experimentally as an analgesic in cases of fibromyalgia. The drug is available in a 5 mg oral preparation or in 2 mg intravenous form. It is marketed by Novartis in Europe, Australia and New Zealand as Navoban, but is not available in the U.S.

Granisetron (1-methyl-N-(9-methyl-9-azabicyclo[3.3.1]non-3-yl)indazole-3-carboxamide) is a serotonin 5-HT₃ receptor antagonist and has the following structure:

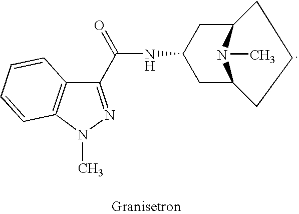

This compound is used as an antiemetic to treat nausea and vomiting following chemotherapy. Its main effect is to reduce the activity of the vagus nerve, which is a nerve that activates the vomiting center in the medulla oblongata. It does not have much effect on vomiting due to motion sickness. This drug does not have any effect on dopamine receptors or muscarinic receptors.

Dolasetron (trade name Anzemet) ((3R)-10-oxo-8-azatricyclo[5.3.1.0^{3,8}]undec-5-yl 1H-indole-3-carboxylate) is a serotonin 5-HT₃ receptor antagonist and has the following structure:

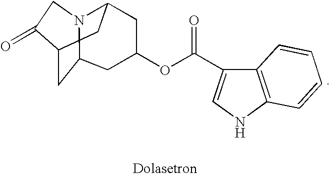

This compound is used to treat nausea and vomiting following chemotherapy. Its main effect is to reduce the activity of the vagus nerve, which is a nerve that activates the vomiting center in the medulla oblongata. It does not have much antiemetic effect when symptoms are due to motion sickness. This drug does not have any effect on dopamine receptors or muscarinic receptors.

Palonosetron (trade name Aloxi) ((3aR)-2-[(3S)-1-azabicyclo[2.2.2]oct-3-yl]-2,3,3a,4,5,6-hexahydro-1H-benz[de]isoquinolin-1-one) is a 5-HT₃ antagonist and has the structure:

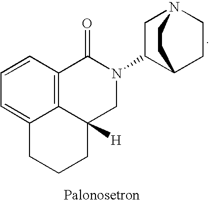

This compound is used in the prevention and treatment of chemotherapy-induced nausea and vomiting (CINV). It is the most effective of the 5-HT₃ antagonists in controlling delayed CINV—nausea and vomiting that appear more than 24 hours after the first dose of a course of chemotherapy—and is the only drug of its class approved for this use by the U.S. Food and Drug Administration.

Alosetron and cilansetron—the latter being developed by Solvay—are not antiemetics; instead, they are indicated in the treatment of a subset of irritable bowel syndrome where diarrhea is the dominant symptom. Alosetron was withdrawn from the U.S. market in 2000 due to unacceptably frequent severe side effects, and is only available through a restrictive program to patients who meet certain requirements.

The disclosure further provides analogs of the 5HT3 antagonists previously described having the common structural Formula (XII):

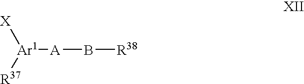

or a salt, hydrate, solvate or N-oxide thereof wherein:

Ar¹ a five or six membered aryl, heteroaryl or cycloalkyl ring;

A is SO₂; C═O; C═S; or C═NR³⁹

B is NH, NR⁴⁰, O, S alkyl, substituted alkyl,

R³⁸ is selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, OR⁴², S(O)_{b}R⁴², NR⁴²R⁴³, CONR⁴²R⁴³, CO₂R⁴², NR⁴²CO₂R⁴³, NR⁴²CONR⁴³R⁴⁴, NR⁴²CSNR⁴³R⁴⁴, NR⁴²C(═NH)NR⁴³R⁴⁴, SO₂NR⁴¹R⁴², NR⁴¹SO₂R⁴², NR⁴¹SO₂NR⁴²R⁴³, P(O)(OR⁴¹)(OR⁴²), and P(O)(R⁴¹)(OR⁴²);

b=0, 1, or 2

R³⁹ is selected from the group consisting of hydrogen, alkyl, and substituted alkyl.

X is selected from the group consisting of hydrogen, halogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, CN, NO₂, OR⁴², S(O)_{b}R⁴², NR⁴²R⁴³, CONR⁴²R⁴³, CO₂R⁴², NR⁴²CO₂R⁴³, NR⁴²CONR⁴³R⁴⁴, NR⁴²CSNR⁴³R⁴⁴, NR⁴²C(═NH)NR⁴³R⁴⁴, SO₂NR⁴¹R⁴², NR⁴¹SO₂R⁴², NR⁴¹SO₂NR⁴²R⁴³, P(O)(OR⁴¹)(OR⁴²), and P(O)(R⁴¹)(OR⁴²);

R³⁷ is selected from the group consisting of hydrogen, halogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, CN, NO₂, OR⁴², S(O)_{b}R⁴², NR⁴²R⁴³, CONR⁴²R⁴³, CO₂R⁴², NR⁴²CO₂R⁴³, NR⁴²CONR⁴³R⁴⁴, NR⁴²CSNR⁴³R⁴⁴, NR⁴²C(═NH)NR⁴³R⁴⁴, SO₂NR⁴¹R⁴², NR⁴¹SO₄₂R⁴², NR⁴¹SO₂NR⁴²R⁴³, P(O)(OR⁴¹)R⁴²), and P(O)(R⁴¹)(OR²);

R⁴⁰-R⁴⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl and substituted heteroarylalkyl or alternatively, R⁴¹ and R⁴², R⁴² and R⁴³, R⁴³ and R⁴⁴, together with the atoms to which they are bonded form a cycloheteroalkyl or substituted cycloheteroalkyl ring,

or alternatively, X and/or at least one R³⁷ together with the atoms to which they are bonded form an aryl, substituted aryl, heteroaryl, substituted heteroaryl, cycloalkyl, substituted cycloalkyl, cycloheteroalkyl or substituted cycloheteroalkyl ring where the ring is optionally fused to another aryl, substituted aryl, heteroaryl, substituted heteroaryl, cycloalkyl, substituted cycloalkyl, cycloheteroalkyl or substituted cycloheteroalkyl ring;

or alternatively, X and/or at least one R³⁸, R⁴¹, R⁴², R⁴³ or R⁴⁴ together with the atoms to which they are bonded form an aryl, substituted aryl, heteroaryl, substituted heteroaryl, cycloalkyl, substituted cycloalkyl, cycloheteroalkyl or substituted cycloheteroalkyl ring where the ring is optionally fused to another aryl, substituted aryl, heteroaryl, substituted heteroaryl, cycloalkyl, substituted cycloalkyl, cycloheteroalkyl or substituted cycloheteroalkyl ring;

In a second aspect the invention provides compounds of structural Formula (XIII) shown below:

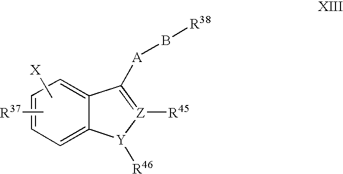

or a salt, hydrate, solvate or N-oxide thereof wherein:

A is SO₂; C═O; C═S; or C═NR³⁹

B is NH, NR⁴⁰, O, S alkyl, substituted alkyl,

R³⁷ is selected from the group consisting of hydrogen, halogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, CN, NO₂, OR², S(O)_{b}R⁴², NR⁴²R⁴³, CONR⁴²R⁴³, CO₂R⁴², NR⁴²CO₂R⁴³, NR⁴²CONR⁴³R⁴⁴, NR⁴²CSNR⁴³R⁴⁴, NR⁴²C(═NH)NR⁴³R⁴⁴, SO₂NR⁴¹R⁴², NR⁴¹SO₂R⁴², NR⁴¹SO₂NR⁴²R⁴³, P(O)(OR⁴¹)(OR⁴²), and P(O)(R⁴¹)(OR²);

X is selected from the group consisting of hydrogen, halogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, CN, NO₂, OR⁴², S(O)_{b}R⁴², NR⁴²R⁴³, CONR⁴²R⁴³, CO₂R⁴², NR⁴²CO₂R⁴³, NR⁴²CONR⁴³R⁴⁴, NR⁴²CSNR⁴³R⁴⁴, NR⁴²C(═NH)NR⁴³R⁴⁴, SO₂NR⁴¹R⁴², NR⁴¹SO₂R⁴², NR⁴¹SO₂NR⁴²R⁴³, P(O)(OR⁴¹)(OR⁴²), and P(O)(R⁴¹)(OR⁴²);

R³⁸ is selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, OR⁴², S(O)_{b}R⁴², NR⁴²R⁴³, CONR⁴²R⁴³, CO₂R⁴², NR⁴²CO₂R⁴³, NR⁴²CONR⁴³R⁴⁴, NR⁴²CSNR⁴³R⁴⁴, NR⁴²C(═NH)NR⁴³R⁴⁴, SO₂NR⁴¹R⁴², NR⁴¹SO₂R⁴², NR⁴¹SO₂NR⁴²R⁴³, P(O)(OR⁴¹)(OR⁴²), and P(O)(R⁴¹)(OR⁴²);

b=0, 1, or 2

R³⁹ is selected from the group consisting of hydrogen, alkyl, and substituted alkyl.

Y and Z are independently selected from N and C

R⁴⁶ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl

Or alternatively, R³⁷ and R⁴⁶ together with the atoms to which they are bonded form an aryl, substituted aryl, heteroaryl, substituted heteroaryl, cycloalkyl, substituted cycloalkyl, cycloheteroalkyl or substituted cycloheteroalkyl ring where the ring is optionally fused to another aryl, substituted aryl, heteroaryl, substituted heteroaryl, cycloalkyl, substituted cycloalkyl, cycloheteroalkyl or substituted cycloheteroalkyl ring;

R⁴⁵ is selected from the group consisting of hydrogen, C₁-C₆ alkyl, and C₁-C₆ substituted alkyl,

or alternatively, R⁴⁵ and B together with the atoms to which they are bonded form an aryl, substituted aryl, heteroaryl, substituted heteroaryl, cycloalkyl, substituted cycloalkyl, cycloheteroalkyl or substituted cycloheteroalkyl ring where the ring is optionally fused to another aryl, substituted aryl, heteroaryl, substituted heteroaryl, cycloalkyl, substituted cycloalkyl, cycloheteroalkyl or substituted cycloheteroalkyl ring;

A preferred embodiment of the invention provides compounds having structural Formula (XIV) shown below:

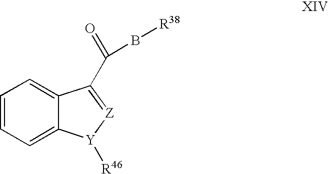

or a salt, hydrate, solvate or N-oxide thereof wherein:

Y and Z are independently selected from N and C

R⁴⁶ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl

B is NH, NR⁴⁰, or O

R³⁸ is selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, OR⁴², S(O)_{b}R⁴², NR⁴²R⁴³, CONR⁴²R⁴³, CO₂R⁴², NR⁴²CO₂R⁴³, NR⁴²CONR⁴³R⁴⁴, NR⁴²CSNR⁴³R⁴⁴, NR⁴²C(═NH)NR⁴³R⁴⁴, SO₂NR⁴¹R⁴², NR⁴¹SO₂R⁴², NR⁴¹SO₂NR⁴²R⁴³, P(O)(OR⁴¹)(OR⁴²), and P(O)(R⁴¹)(OR⁴²);

Some preferred embodiments provide compounds as described above wherein, R³⁸, forms a ring that can be fused with additional substituted or unsubstituted rings. non-limiting examples of such a ring includes groups having the formula (a), (b), and (c) below:

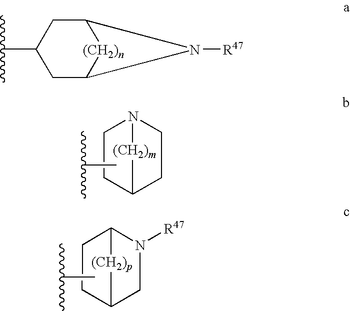

Wherein n, m and p are independently selected from 1, 2 or 3.

R⁴⁷ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)_{q}R⁴⁸ Where q is 1, 2 or 3 and R⁴⁸ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

Some even more preferred embodiments provide compounds as described above wherein, R³⁸ forms a ring that can be fused with additional substituted or unsubstituted rings and can comprise at least one double bond. Preferred embodiments of such a ring includes groups having the formula:

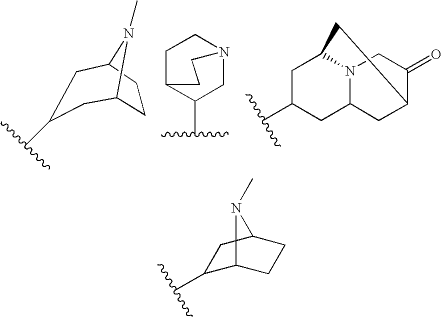

And their stereoisomers

A more preferred embodiment of the invention provides compounds having structural Formula (XV) shown below:

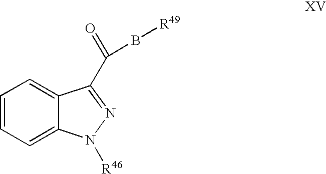

### B is NH, NR⁵⁰, or O

R⁴⁶ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl
each R⁴⁹ is independently selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, and substituted heteroarylalkyl.

R⁵⁰ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl,

Some preferred embodiments provide compounds as described above wherein, R⁴⁹, forms a ring that can be fused with additional substituted or unsubstituted rings. non-limiting examples of such a ring includes groups having the formula (a), (b), and (c) below:

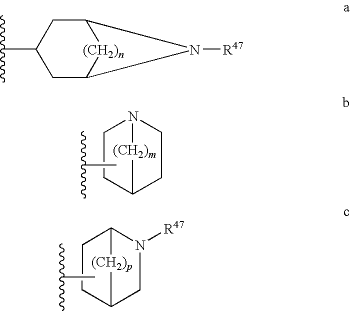

Wherein n, m and p are independently selected from 1, 2 or 3.

R⁴⁷ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)_{q}R⁴⁸ Where q is 1, 2 or 3 and R⁴⁸ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

Some even more preferred embodiments provide compounds as described above wherein, R⁴⁹ forms a ring that can be fused with additional substituted or unsubstituted rings and can comprise at least one double bond. Preferred embodiments of such a ring system includes groups having the formula:

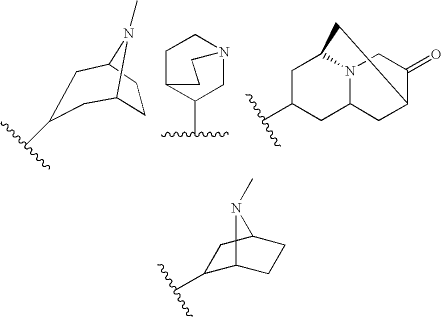

And their stereoisomers

An especially preferred aspect the invention provides a compound having the structure below:

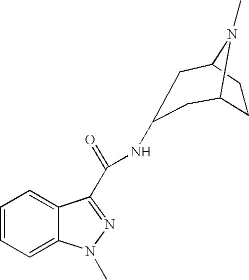

or a salt, hydrate, solvate or N-oxide thereof

Another preferred embodiment of the invention provides compounds having structural Formula (XVI) shown below:

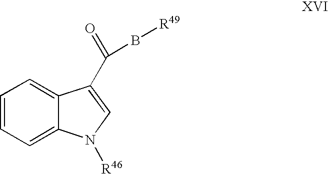

### B is NH, NR⁵⁰, or O

R⁴⁶ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl
each R⁴⁹ is independently selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, and substituted heteroarylalkyl.

R⁵⁰ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl,

Some preferred embodiments provide compounds as described above wherein, R⁴⁹, forms a ring that can be fused with additional substituted or unsubstituted rings. non-limiting examples of such a ring includes groups having the formula (a), (b), and (c) below:

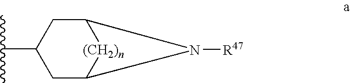

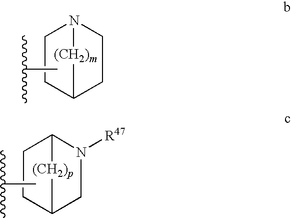

Wherein n, m and p are independently selected from 1, 2 or 3.

R⁴⁷ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)_{q}R⁴⁸ Where q is 1, 2 or 3 and R⁴⁸ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

Some even more preferred embodiments provide compounds as described above wherein, R⁴⁹ forms a ring that can be fused with additional substituted or unsubstituted rings and can comprise at least one double bond. Preferred embodiments of such a ring system includes groups having the formula:

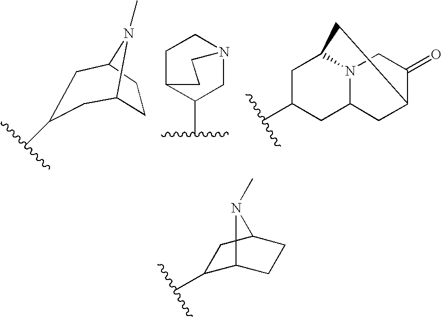

And their stereoisomers

Another especially preferred aspect the invention provides compounds having the structure below:

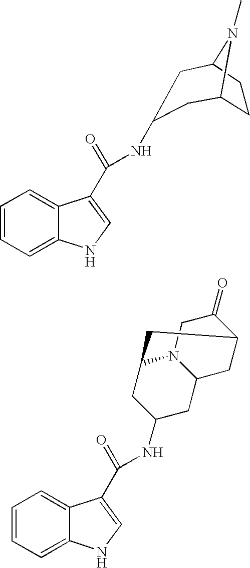

or a salt, hydrate, solvate or N-oxide thereof.

Another preferred embodiment of the invention provides compounds having structural Formula (XVII) shown below:

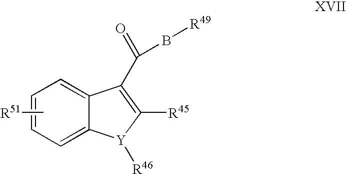

or a salt, hydrate, solvate or N-oxide thereof wherein:

Y is independently selected from N and C
R⁴⁶ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl
R⁵¹, is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₃-C₆ cycloalkyl-C₁-C₆ substituted alky, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl
R⁴⁵ is selected from the group consisting of hydrogen, C₁-C₆ alkyl, and C₁-C₆ substituted alkyl,
or alternatively, R⁴⁶ and R⁵¹ together with the atoms to which they are bonded form an aryl, substituted aryl, heteroaryl, substituted heteroaryl, cycloalkyl, substituted cycloalkyl, cycloheteroalkyl or substituted cycloheteroalkyl ring.

B is NH, NR⁴⁰, or CR⁵²R⁵³
Wherein R⁵² and R⁵³ are independently selected from the group consisting of hydrogen, C₁-C₆ alkyl, and C₁-C₆ substituted alkyl, C₃-C₆ cycloalkyl, C₃-C₆ cycloalkyl-C₁-C₆ alkyl.
alternatively, R⁴⁵ and B together with the atoms to which they are bonded form an aryl, substituted aryl, heteroaryl, substituted heteroaryl, cycloalkyl, substituted cycloalkyl, cycloheteroalkyl or substituted cycloheteroalkyl ring.
And R⁴⁹ is selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, and substituted heteroarylalkyl.

A preferred embodiment of the invention provides compounds having structural Formula (XVII) as described above where R⁴⁶ is selected from the group consisting of hydrogen, C₁-C₆ alkyl, and C₁-C₆ substituted alkyl, C₃-C₆ cycloalkyl, C₁-C₆ alkenyl, aryl, and arylalkyl, and R⁴⁹ is a substituted alky group as shown in the formula below:

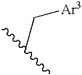

Where Ar³ is a substituted or unsubstituted five or six membered aryl, or heteroaryl ring.

Another more preferred embodiment of the invention provides compounds having structural Formula (XVII) as described above where R⁴⁶ is selected from the group consisting of hydrogen, C₁-C₆ alkyl, and C₁-C₆ substituted alkyl, C₃-C₆ cycloalkyl, C₁-C₆ alkenyl, aryl, and arylalkyl, and R⁴⁹ is a substituted alky group as shown in the formula below:

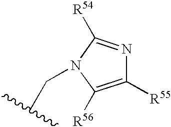

Where one of the groups R⁵⁴, R⁵⁵ and R⁵⁶ are selected from the group consisting of hydrogen, C₁-C₆ alkyl, C₂-C₆ alkenyl, C₃-C₇ cycloalkyl, phenyl, phenyl C₁-C₃ alkyl and each of the remaining 2 groups may be the same or different and are selected from hydrogen, and C₁-C₆ alkyl.

Another even more preferred embodiment of the invention provides compounds having structural Formula (XVII) as described above where R⁴⁶ is selected from the group consisting of hydrogen, C₁-C₆ alkyl, and C₁-C₆ substituted alkyl, C₃-C₆ cycloalkyl, C₁-C₆ alkenyl, aryl, and arylalkyl, and R⁴⁹ is a substituted alky group as shown in the formula below:

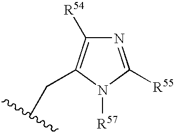

Where one of the groups R⁵⁴ and R⁵⁵ are independently selected from the group consisting of hydrogen, C₁-C₆ alkyl, C₂-C₆ alkenyl, C₃-C₇ cycloalkyl, phenyl, phenyl C₁-C₃ alkyl and the remaining group may be the same or different and are selected from hydrogen, and C₁-C₆ alkyl.

R⁵⁷ is selected from hydrogen, C₁-C₆ alkyl, or C₁-C₆ substituted alkyl

Another even more preferred embodiment of the invention provides compounds having structural Formula (XVIII) shown below or a salt, hydrate, solvate or N-oxide thereof wherein:

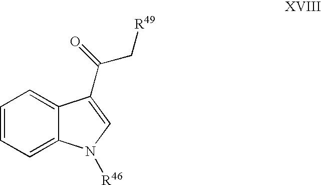

R⁴⁶ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl
And R⁴⁹ is selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, and substituted heteroarylalkyl.

An even more preferred embodiment of the invention provides compounds having structural Formula (XVIII) as described above where R⁴⁶ is selected from the group consisting of hydrogen, C₁-C₆ alkyl, and C₁-C₆ substituted alkyl and R⁴⁹ is a substituted alky group as shown in the formula below:

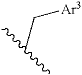

Where Ar³ is a substituted or unsubstituted five or six membered aryl, or heteroaryl ring.

Another even more preferred embodiment of the invention provides compounds having structural Formula (XVIII) as described above where R⁴⁶ is selected from the group consisting of hydrogen, C₁-C₆ alkyl, and C₁-C₆ substituted alkyl and R⁴⁹ is a substituted alky group as shown in the formula below:

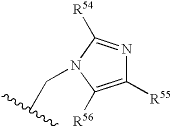

Where one of the groups R⁵⁴, R⁵⁵ and R⁵⁶ are selected from the group consisting of hydrogen, C₁-C₆ alkyl, C₂-C₆ alkenyl, C₃-C₇ cycloalkyl, phenyl, phenyl C₁-C₃ alkyl and each of the remaining 2 groups may be the same or different and are selected from hydrogen, and C₁-C₆ alkyl.

Another especially preferred embodiment of the invention provides compounds having structural Formula (XVIII) as described above where R⁴⁶ is selected from the group consisting of hydrogen, C₁-C₆ alkyl and R⁴⁹ is a substituted alky group as shown in the formula below:

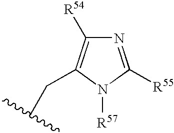

Where one of the groups R⁵⁴ and R⁵⁵ are independently selected from the group consisting of hydrogen, C₁-C₆ alkyl, C₂-C₆ alkenyl, C₃-C₇ cycloalkyl, phenyl, phenyl C₁-C₃ alkyl and the remaining group may be the same or different and are selected from hydrogen, and C₁-C₆ alkyl.

R⁵⁷ is selected from hydrogen, C₁-C₆ alkyl, or C₁-C₆ substituted alkyl

In yet another especially preferred embodiment of the invention provides a compound having the structure below:

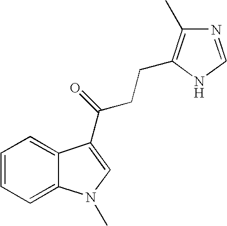

or a salt, hydrate, solvate or N-oxide thereof.

Yet another preferred embodiment of the invention provides compounds having structural Formula (XIX) shown below wherein:

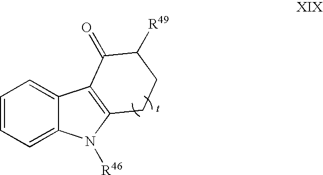

or a salt, hydrate, solvate or N-oxide thereof wherein:
R⁴⁶ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, C₁-C₆ alkenyl, C₁-C₆ substituted alkenyl, C₁-C₆ alkynyl, C₁-C₆ substituted alkynyl, aryl, arylalkyl, heteroaryl, heteroarylalkyl
T is 0, 1 or 2 and
And R⁴⁹ is selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, and substituted heteroarylalkyl.

A more preferred embodiment of the invention provides compounds having structural Formula (XIX) as described above where R⁴⁶ is selected from the group consisting of hydrogen, C₁-C₆ alkyl, and C₁-C₆ substituted alkyl and R⁴⁹ is a substituted alky group as shown in the formula below:

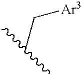

Where Ar³ is a substituted or unsubstituted five or six membered aryl, or heteroaryl ring.

Another even more preferred embodiment of the invention provides compounds having structural Formula (XIX) as described above where R⁴⁶ is selected from the group consisting of hydrogen, C₁-C₆ alkyl, and C₁-C₆ substituted alkyl and R⁴⁹ is a substituted alky group as shown in the formula below:

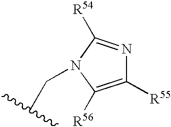

Where one of the groups R⁵⁴, R⁵⁵ and R⁵⁶ are selected from the group consisting of hydrogen, C₁-C₆ alkyl, C₂-C₆ alkenyl, C₃-C₇ cycloalkyl, phenyl, phenyl C₁-C₃ alkyl and each of the remaining 2 groups may be the same or different and are selected from hydrogen, and C₁-C₆ alkyl.

Another even more preferred embodiment of the invention provides compounds having structural Formula (XIX) as described above where R⁴⁶ is selected from the group consisting of hydrogen, C₁-C₆ alkyl and R⁴⁹ is a substituted alky group as shown in the formula below:

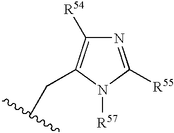

Where one of the groups R⁵⁴ and R⁵⁵ are independently selected from the group consisting of hydrogen, C₁-C₆ alkyl, C₂-C₆ alkenyl, C₃-C₇ cycloalkyl, phenyl, phenyl C₁-C₃ alkyl and the remaining group may be the same or different and are selected from hydrogen, and C₁-C₆ alky.

R⁵⁷ is selected from hydrogen, C₁-C₆ alkyl, or C₁-C₆ substituted alkyl

Yet another especially preferred embodiment of the invention provides compounds having structural Formula (XX) shown below or a salt, hydrate, solvate or N-oxide thereof wherein:

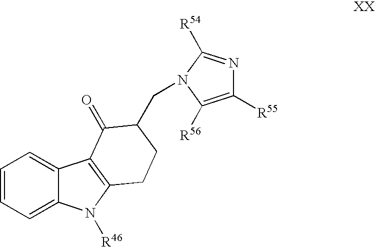

R⁴⁶ is selected from the group consisting of hydrogen, C₁-C₆ alkyl
Where one of the groups R⁵⁴, R⁵⁵ and R⁵⁶ are selected from the group consisting of hydrogen, C₁-C₆ alkyl, C₂-C₆ alkenyl, C₃-C₇ cycloalkyl, phenyl, phenyl C₁-C₃ alkyl and each of the remaining 2 groups may be the same or different and are selected from hydrogen, and C₁-C₆ alkyl.

In yet another especially preferred aspect the invention provides a compound having the structure below:

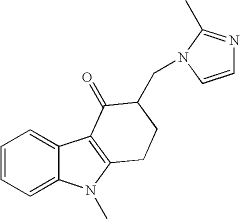

or a salt, hydrate, solvate or N-oxide thereof.

Yet another preferred embodiment of the invention provides compounds having structural Formula (XXI) shown below or a salt, hydrate, solvate or N-oxide thereof wherein:

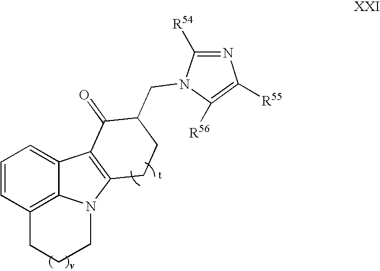

t and v are independently selected from 0, 1 or 2 and
Where one of the groups R⁵⁴, R⁵⁵ and R⁵⁶ are selected from the group consisting of hydrogen, C₁-C₆ alkyl, C₂-C₆ alkenyl, C₃-C₇ cycloalkyl, phenyl, phenyl C₁-C₃ alkyl and each of the remaining 2 groups may be the same or different and are selected from hydrogen, and C₁-C₆ alkyl.

Another even more preferred embodiment of the invention provides compounds having structural Formula (XXI) as described above where t and v are both equal to one and where one of the groups R⁵⁴, R¹⁵ and R⁵⁶ are selected from the group consisting of hydrogen, C₁-C₆ alkyl, C₂-C₆ alkenyl, C₃-C₇ cycloalkyl, phenyl, phenyl C₁-C₃ alkyl and each of the remaining 2 groups may be the same or different and are selected from hydrogen, and C₁-C₆ alkyl

In yet another especially preferred embodiment of the invention provides a compound having the structure below:

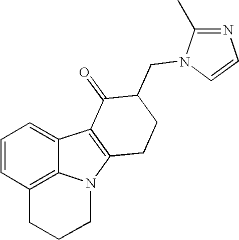

or a salt, hydrate, solvate or N-oxide thereof.

In yet another embodiment of the invention provides compounds having the structure formula (XXII) below or a salt, hydrate, solvate or N-oxide thereof wherein:

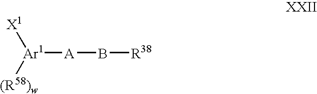

Ar¹ a five or six membered aryl, heteroaryl or cycloalkyl ring;

A is SO₂; C═O; or C═NR³⁹

B is NH, NR⁴⁰, O, S, Alkyl, substituted alkyl

### W is 0, 1, 2, 3, or 4

R³⁸ is selected from the group consisting of hydrogen, alkyl, substituted alkyl, aryl, substituted aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, OR⁴², S(O)_{b}R⁴², NR⁴²R⁴³, CONR⁴²R⁴³, CO₂R⁴², NR⁴²CO₂R⁴³, NR⁴²CONR⁴³R⁴⁴, NR⁴²CSNR⁴³R⁴⁴, NR⁴²C(═NH)NR⁴³R⁴⁴, SO₂NR⁴¹R⁴², NR⁴¹SO₂R⁴², NR⁴¹SO₂NR⁴²R⁴³, P(O)(OR⁴¹)(OR⁴²), and P(O)(R⁴¹)(OR⁴²);

b=0, 1, or 2

R³⁹ is selected from the group consisting of hydrogen, alkyl, and substituted alkyl.

X¹ is selected from the group consisting of hydrogen, halogen, perfluoroalkyl, perfluoroalkoxy, alkyl, alkenyl, alkynyl, hydroxy, oxo, mercapto, alkylthio, alkoxy, aryl or heteroaryl, aryloxy or heteroaryloxy, arylalkyl or heteroarylalkyl, arylalkoxy or heteroarylalkoxy, amino, alkyl- and dialkylamino groups, carbamoyl, alkylcarbonyl, carboxyl, alkoxycarbonyl, alkylaminocarbonyl, dialkylamino carbonyl, arylcarbonyl, aryloxycarbonyl, alkylsulfonyl, arylsulfonyl, cycloalkyl, cyano, C₁-C₆ alkylthio, arylthio, nitro, keto, acyl, phosphate or phosphonyl, sulfamyl, sulfonyl, sulfinyl, and combinations thereof.

Each R⁵⁸ is independently is selected from the group consisting of hydrogen, halogen, perfluoroalkyl, perfluoroalkoxy, alkyl, alkenyl, alkynyl, hydroxy, oxo, mercapto, alkylthio, alkoxy, aryl or heteroaryl, aryloxy or heteroaryloxy, arylalkyl or heteroarylalkyl, arylalkoxy or heteroarylalkoxy, amino, alkyl- and dialkylamino groups, carbamoyl, alkylcarbonyl, carboxyl, alkoxycarbonyl, alkylaminocarbonyl, dialkylamino carbonyl, arylcarbonyl, aryloxycarbonyl, alkylsulfonyl, arylsulfonyl, cycloalkyl, cyano, C₁-C₆ alkylthio, arylthio, nitro, keto, acyl, phosphate or phosphonyl, sulfamyl, sulfonyl, sulfinyl, and combinations thereof.

R⁴⁰-R⁴⁴ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl and substituted heteroarylalkyl or alternatively, R⁴¹ and R⁴², R⁴² and R⁴³, R⁴³ and R⁴⁴, together with the atoms to which they are bonded form a cycloheteroalkyl or substituted cycloheteroalkyl ring;

or alternatively, X¹ and/or at least one R⁵⁸ together with the atoms to which they are bonded form an aryl, substituted aryl, heteroaryl, substituted heteroaryl, cycloalkyl, substituted cycloalkyl, cycloheteroalkyl or substituted cycloheteroalkyl ring where the ring is optionally fused to another aryl, substituted aryl, heteroaryl, substituted heteroaryl, cycloalkyl, substituted cycloalkyl, cycloheteroalkyl or substituted cycloheteroalkyl ring;

or alternatively, X¹ and/or at least one R³⁸, R⁴⁰, R⁴¹, R⁴², R⁴³ or R⁴⁴ together with the atoms to which they are bonded form an aryl, substituted aryl, heteroaryl, substituted heteroaryl, cycloalkyl, substituted cycloalkyl, cycloheteroalkyl or substituted cycloheteroalkyl ring where the ring is optionally fused to another aryl, substituted aryl, heteroaryl, substituted heteroaryl, cycloalkyl, substituted cycloalkyl, cycloheteroalkyl or substituted cycloheteroalkyl ring;

In yet another preferred embodiment of the invention provides compounds having the structure formula (XXIII) below:

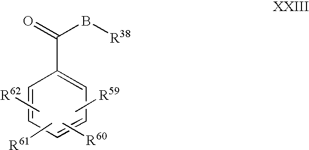

Where B is NH or O and R⁵⁹, R⁶⁰, R⁶¹ and R⁶² are independently selected from hydrogen, halogen alkyl, alkoxy, amino, acylamino, hydroxyl or nitro and R³⁸ forms a ring that can be fused with additional substituted or unsubstituted rings. non-limiting examples of such a ring includes groups having the formula d, e, and f below:

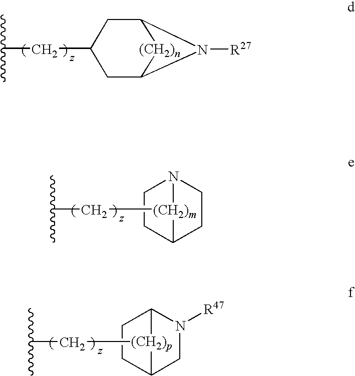

Wherein n, m, p and z are independently selected from 0, 1, 2 or 3.
R⁴⁷ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)_{q}R⁴⁸ Where q is 1, 2 or 3 and R⁴⁸ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

In yet another more preferred embodiment of the invention provides compounds having the structure formula (XXIV) below or a salt, hydrate, solvate or thereof wherein:

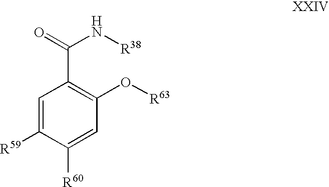

R⁵⁹ is hydrogen, chloro or bromo, R⁶⁰ is hydrogen or amino, R⁶³ is selected from C₁-C₆ alkyl, C₁-C₆ substituted alkyl, C₃-C₆ cycloalkyl, C₃-C₆ cycloalkyl-C₁-C₆ alkyl and R³⁸ forms a ring that can be fused with additional substituted or unsubstituted rings. non-limiting examples of such a ring includes groups having the formula d, e, and f below:

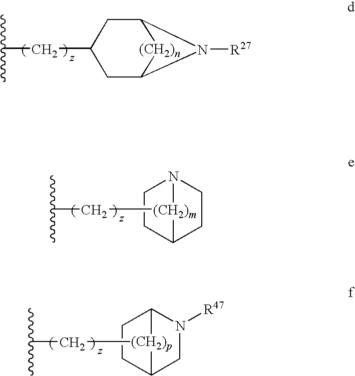

Wherein n, m, p and z are independently selected from 0, 1, 2 or 3.
R⁴⁷ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)_{q}R⁴⁸ Where q is 1, 2 or 3 and R⁴⁸ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

An especially more preferred embodiment of the invention provides compounds having the structure formula (XXIV) above or a salt, hydrate, solvate or thereof wherein R⁵⁹ is hydrogen, chloro or bromo, R⁶⁰ is hydrogen or amino, R⁶³ is selected from methyl or 1-(methylsulfinyl)ethyl and R³⁸ is either of the formula e above where Z is 0 and m is 2, C₁-C₆ alkyl, and C₁-C₆ substituted alkyl,

In yet another especially preferred embodiment of the invention provides compounds having the structure:

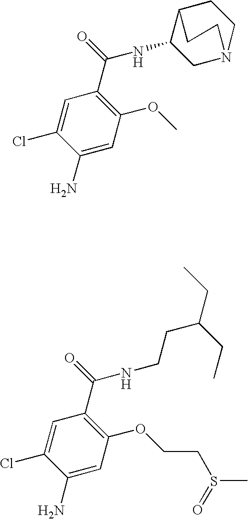

or a salt, hydrate, solvate or N-oxide thereof.

In yet another more preferred embodiment of the invention provides compounds having the structure formula (XXV) below or a salt, hydrate, solvate or thereof wherein:

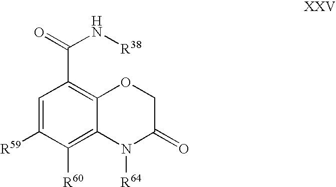

R⁵⁹ is hydrogen, chloro or bromo, R⁶⁰ is hydrogen or amino, R⁶⁴ is selected from hydrogen, C₁-C₆ alkyl, and C₁-C₆ substituted alkyl, C₃-C₆ cycloalkyl, C₃-C₆ cycloalkyl-C₁-C₆ alkyl and R³⁸ forms a ring that can be fused with additional substituted or unsubstituted rings. non-limiting examples of such a ring includes groups having the formula d, a, and f below:

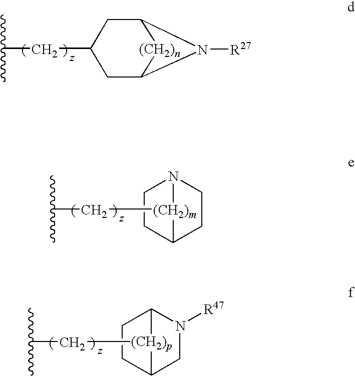

Wherein n, m, p and z are independently selected from 0, 1, 2 or 3.
R⁴⁷ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)_{q}R⁴⁸ Where q is 1, 2 or 3 and R⁴⁸ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

In yet another especially preferred embodiment of the invention provides a compound having the structure:

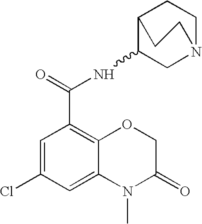

or a salt, hydrate, solvate or N-oxide thereof.

In yet another especially preferred embodiment of the invention provides compounds having the structure formula (XXVI) below or a salt, hydrate, solvate or thereof wherein:

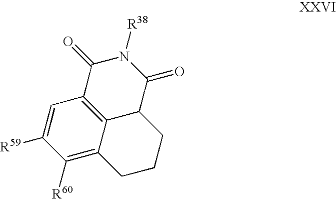

R⁵⁹ is hydrogen, chloro or bromo, R⁶⁰ is hydrogen or amino, and R³⁸ forms a ring that can be fused with additional substituted or unsubstituted rings. non-limiting examples of such a ring includes groups having the formula d, e, and f below:

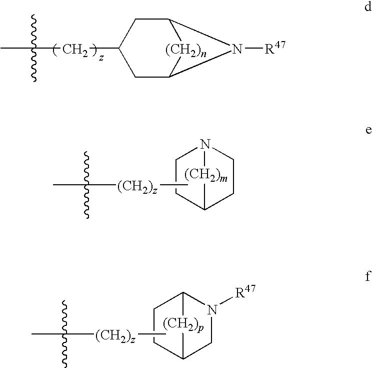

Wherein n, m, p and z are independently selected from 0, 1, 2 or 3.
R⁴⁷ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)_{q}R⁴⁸ Where q is 1, 2 or 3 and R⁴⁸ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

In yet another especially preferred embodiment of the invention provides a compound having the structure:

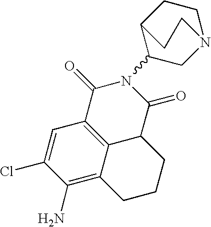

or a salt, hydrate, solvate or N-oxide thereof.

In yet another more preferred embodiment of the invention provides compounds having the structure formula (XXVII) below or a salt, hydrate, solvate or thereof wherein:

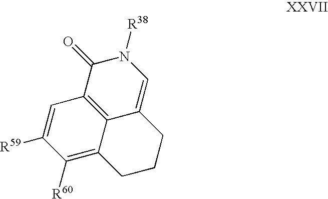

R⁵⁹ is hydrogen, chloro or bromo, R⁶⁰ is hydrogen or amino, and R³⁸ forms a ring that can be fused with additional substituted or unsubstituted rings. non-limiting examples of such a ring includes groups having the formula d, e, and f below:

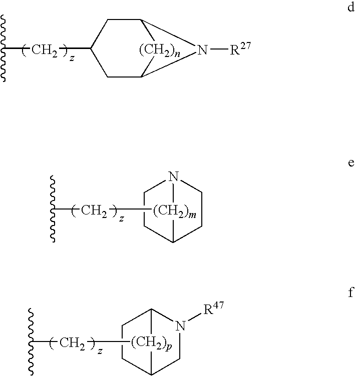

Wherein n, m, p and z are independently selected from 0, 1, 2 or 3.
R⁴⁷ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)_{q}R⁴⁸ Where q is 1, 2 or 3 and R⁴⁸ is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

In yet another especially preferred embodiment of the invention provides a compound having the structure:

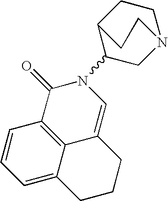

or a salt, hydrate, solvate or N-oxide thereof

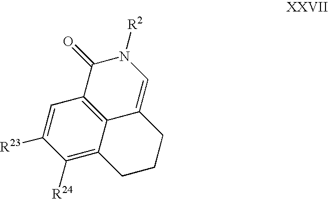

or a salt, hydrate, solvate or thereof wherein:

R²³ is hydrogen, chloro or bromo, R²⁴ is hydrogen or amino, and R² forms a ring that can be fused with additional substituted or unsubstituted rings. non-limiting examples of such a ring includes groups having the formula d, e, and f below:

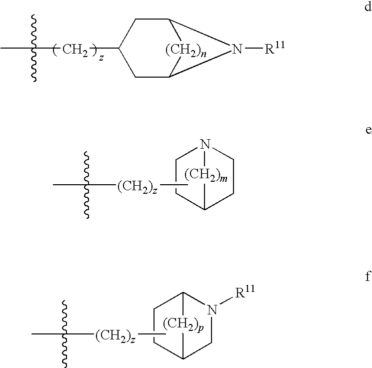

wherein n, m, p and z are independently selected from 0, 1, 2 or 3;

R¹¹ is selected from the group consisting of hydrogen, C₁-C₆ alkyl, and C₁-C₆ substituted alkyl, C₃-C₆ cycloalkyl, C₃-C₆ cycloalkyl-C₁-C₆ alkyl, or a group (CH₂)_{q}R¹² Where q is 1, 2 or 3 and R¹² is thienyl, pyrrolyl, furyl or imidazolyl optionally substituted by one or 2 substituents selected from Halogen, C₁-C₆ alkyl, C₁-C₆ alkoxy, Substituted C₁-C₆ alkyl, aryl or substituted aryl.

In another aspect, the disclosure provides a compound having the structure:

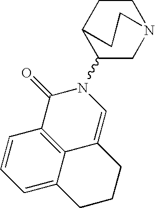

or a salt, hydrate, solvate or N-oxide thereof.

A phosphodiesterase inhibitor is a drug that blocks one or more of the five subtypes of the enzyme phosphodiesterase (PDE), therefore preventing the inactivation of the intracellular second messengers, cyclic adenosine monophosphate (cAMP) and cyclic guanosine monophosphate (cGMP), by the respective PDE subtype(s). Non-selective phosphodiesterase inhibitors include the minor stimulant caffeine; the bronchodilator theophylline; and IBMX (3-isobutyl-1-methylxanthine): used as investigative tool in pharmacological research. PDE1-selective inhibitors include Vinpocetine; PDE2-selective inhibitors include EHNA; PDE3-selective inhibitors include Enoximone and Milrinone: used clinically for short-term treatment of cardiac failure. These drugs mimic sympathetic stimulation and increase cardiac output. PDE3 is sometimes referred to as cGMP-inhibited phosphodiesterase. PDE4-selective inhibitors include Mesembrine: an alkaloid present in the herb Sceletium tortuosum; Rolipram: used as investigative tool in pharmacological research; and Ibudilast, a neuroprotective and bronchodilator drug used mainly in the treatment of asthma and stroke and has the following structure:

Ibudilast (AV-411) (2-methyl-1-(2-propan-2-ylpyrazolo[1,5-a]pyridin-3-yl)propan-1-one) has the following structure:

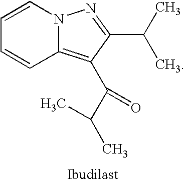

This compound is an antiinflammatory drug used mainly in Japan, which acts as a phosphodiesterase inhibitor, inhibiting the PDE-4 subtype to the greatest extent, but also showing significant inhibition of other PDE subtypes. Ibudilast has bronchodilator, vasodilator, and neuroprotective effects, and is mainly used in the treatment of asthma and stroke. It inhibits platelet aggregation, and may also be useful in the treatment of multiple sclerosis. Ibudilast crosses the blood-brain barrier and suppresses glial cell activation. This activity has been shown to make ibudilast useful in the treatment of neuropathic pain and it not only enhances analgesia produced by opioid drugs, but also reduces the development of tolerance.

Ibudilast inhibits PDE-4 to the greatest extent, but also shows significant inhibition of other PDE subtypes, and so can be viewed either as a selective PDE-4 inhibitor or a non-selective phosphodiesterase inhibitor depending on the dose used. PDE4 is the major cAMP-metabolizing enzyme found in inflammatory and immune cells. PDE4 inhibitors have proven potential as anti-inflammatory drugs especially in airway diseases. They suppress the release of inflammatory signals, e.g., cytokines, and inhibit the production of reactive oxygen species. PDE4 inhibitors have a high therapeutic and commercial potential as non-steroidal disease controllers in inflammatory airway diseases such as asthma, COPD and rhinitis. PDE4 inhibitors may have an antidepressant action and have also recently been proposed for use as antipsychotic medications.

PDE5-selective inhibitors include Sildenafil, tadalafil, vardenafil; and the newer ones, udenafil and avanafil: selectively inhibit (PDE5), which is cGMP-specific and responsible for the degradation of cGMP in the corpus cavernosum. These phosphodiesterase inhibitors are used primarily as remedies for erectile dysfunction, as well as having some other medical applications such as treatment of pulmonary hypertension.

Antiviral drugs are a class of medication used specifically for treating viral infections. Like antibiotics, specific antivirals are used for specific viruses. Antiviral drugs are one class of antimicrobials, a larger group which also includes antibiotic, antifungal, and antiparasitic drugs. They are relatively harmless to the host, and therefore can be used to treat infections.

Methylphenidate (MPH) (Methyl 2-phenyl-2-(2-piperidyl)acetate) and has the following structure:

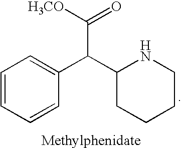

This compound is a prescription stimulant commonly used to treat Attention-deficit hyperactivity disorder, or ADHD. Methylphenidate is a dopamine reuptake inhibitor, which means that it increases the level of the dopamine neurotransmitter in the brain by partially blocking the transporters that remove it from the synapses. It is also one of the primary drugs used to treat the daytime drowsiness symptoms of narcolepsy and chronic fatigue syndrome. The drug is seeing early use to treat cancer-related fatigue. Brand names of drugs that contain methylphenidate include Ritalin (Ritalina, Rilatine, Attenta, Methylin, Penid, Rubifen); and the sustained release tablets Concerta, Metadate CD, Ritalin LA, and Ritalin-SR. Focalin is a preparation containing only dextro-methylphenidate, rather than the usual racemic dextro- and levo-methylphenidate mixture of other formulations. A newer way of taking methylphenidate is by using a transdermal patch (under the brand name Daytrana), similar to those used for hormone replacement therapy, nicotine release and pain relief (fentanyl) dopamine modulator is methylphenidate.

Another especially preferred embodiment of the invention includes analogs of methylphenidate having the following formulas:

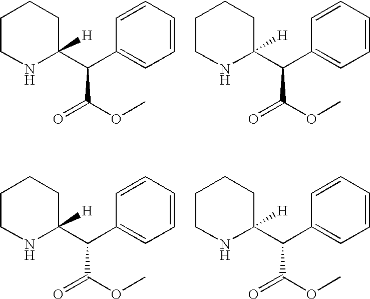

or a salt, hydrate, solvate or N-oxide thereof or a derivative there of which is a prodrug.

Phosphorylation via protein kinases is responsible for a large part of cellular signal transduction and is described as a universal regulatory mechanism. Perturbation of kinase-mediated signaling pathways results in a number of diseases, including diabetes, cancer, and inflammation. Because most protein kinases reside in the cell in an inactive state and are activated by signal transduction processes, many diseases are triggered by overactivation of protein kinases via mutation, overexpression, or malfunctioning cellular inhibition. The human genome encodes some 518 protein kinases that are notably different in how their catalysis is regulated but share a catalytic domain conserved in sequence and structure. The latter consists of 250-300 amino acids, binds substrate and cosubstrate, and catalyzes the phosphorylation reaction. This catalytic domain, together with less conserved surrounding sites, has been the focus of inhibitor design that has exploited differences in kinase structure and pliability to achieve selectivity.

Many drugs that target protein kinases are in clinical trials, and some have already been approved, such as the Abl kinase inhibitor Gleevec for therapy against chronic myelogenous leukemia and Tarceva (erlotinib) against nonsmall cell lung cancer. The first protein kinase inhibitor that passed the clinical phase was fasudil (HA1077) in 1995 as a treatment for cerebral vasospasm.

Fasudil (CAS103745-39-7) (1-(5-Isoquinolinesulfonyl)homopiperazine dihydrochloride) has the following structure:

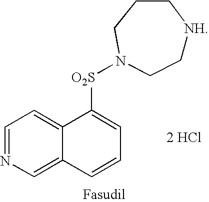

This compounds has a significant vasodilatory effect attributed to its inhibition of Rho-kinase signaling to myosin light-chain kinase and is in clinical trials for the treatment of angina pectoris. Apart from its pharmacological role in cerebral vasospasm and angina pectoris, Rho-kinase plays an important role in cell division, differentiation, apoptosis, transformation, and the invasion and migration of cancer cells.

The α₂ receptor is a type of adrenergic receptor. The adrenergic receptors (or adrenoceptors) are a class of G protein-coupled receptors that are targets of the catecholamines. Adrenergic receptors specifically bind their endogenous ligands, the catecholamines adrenaline, and noradrenaline (epinephrine and norepinephrine), and are activated by these. Many cells possess these receptors, and the binding of an agonist will generally cause a sympathetic response (i.e., the fight-or-flight response). For instance, the heart rate will increase and the pupils will dilate, energy will be mobilized, and blood flow diverted from other, non-essential, organs to skeletal muscle (note: Sympathetic activity will result in vasodilation of coronary arteries via the β₂-adrenergic receptors.). It is a specific object of the invention to provide the compounds which can be represented by the following formulae.

Analogs of fasudil are represented by structural Formula (XXVIII) is provided:

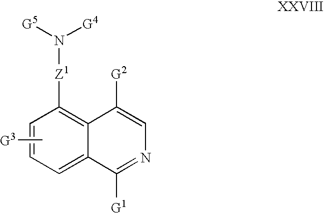

or a salt, hydrate, solvate or N-oxide thereof or a derivative thereof which is a prodrug wherein:

G¹ is selected from the group consisting of hydrogen, halogen, hydroxy, OR⁶, or NR⁶R⁷

G² is selected from the group consisting of hydrogen, halogen, hydroxy, cyano, carboxy, alkyl substituted alkyl, alkenyl, substituted alkenyl, alkynyl substituted alkynyl, aryl, substituted aryl, heteroalkyl, substituted heteroalkyl, heteroaryl, substituted heteroaryl, OR⁶, S(O)_{b}R⁶, NR⁶R⁷, SO₂NR⁶R⁷, NR⁶SO₂R⁷, NR⁶SO₂NR⁷R⁸, P(O)(OR⁶)(OR⁷), and P(O)(R⁶)(OR⁷);

b=0, 1, or 2

R⁶-R⁸ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl and substituted heteroarylalkyl or alternatively, R⁶ and R⁷, R⁶ and R⁸, or R⁷ and R⁸, together with the atoms to which they are bonded form a cycloheteroalkyl or substituted cycloheteroalkyl ring;

G³ is selected from the group consisting of hydrogen, halogen, hydroxy, cyano, alkyl, substituted alkyl, alkenyl, substituted alkenyl, alkynyl substituted alkynyl, aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, OR⁶, S(O)_{b}R⁶, NR⁶R⁷, CONR⁶R⁷, CO₂R⁶, NR⁶CO₂R⁷, NR⁶CONR⁷R⁸, NR⁶CSNR⁷R⁸, NR⁶C(═NH)NR⁷R⁸, SO₂NR⁶R⁷, NR⁶SO₂R⁷, NR⁶SO₂NR⁷R⁸, P(O)(OR⁶)(OR⁷), and P(O)(R⁶)(OR⁷); or an aryl group which may be substituted (provided that G3 substitutes at the 3-, 6-, 7-, or 8-position of the isoquinoline ring); with R⁶-R⁸ as defined above

G⁴ and G⁵ are independently selected from hydrogen, C₁-C₈ alkyl, and C₁-C₈ substituted alkyl, C₃-C₈ cycloalkyl, C₃-C₈ cycloalkyl-C₁-C₆ alkyl or G⁴ and Gs together with the atoms to which they are bonded form a cycloheteroalkyl or substituted cycloheteroalkyl ring;

Z¹ is selected from SO₂ or C(O)

A preferred embodiment of the invention provides compounds having structural Formula (XXIX) shown below:

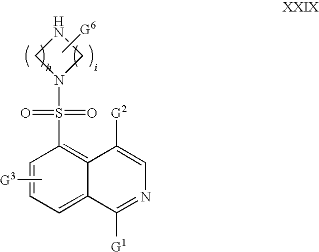

or a salt, hydrate, solvate or N-oxide thereof or a derivative thereof which is a prodrug wherein:

G¹ is selected from the group consisting of hydrogen, halogen, hydroxy, OR⁶, or NR⁶R⁷

G² is selected from the group consisting of hydrogen, halogen, hydroxy, cyano, carboxy, C₁-C₈ alkyl, C₁-C₈ alkenyl, C₁-C₈ alkynyl

G³ is selected from the group consisting of hydrogen, halogen, hydroxyl, cyano, alkyl, substituted alkyl, alkenyl, substituted alkenyl, alkynyl substituted alkynyl, aryl, arylalkyl, substituted arylalkyl, acyl, substituted acyl, heteroalkyl, substituted heteroalkyl, heteroaryl, substituted heteroaryl, heteroarylalkyl, substituted heteroarylalkyl, OR⁶, S(O)_{b}R⁶, NR⁶R⁷, CONR⁶R⁷, CO₂R⁶, NR⁶CO₂R⁷, NR⁶CONR⁷R⁸, NR⁶CSNR⁷R⁸, NR⁶C(═NH)NR⁷R⁸, SO₂NR⁶R⁷, NR⁶SO₂R⁷, NR⁶SO₂NR⁷R⁸, P(O)(OR⁶)(OR⁷), and P(O)(R⁶)(OR⁷); or an aryl group which may be substituted (provided that G3 substitutes at the 3-, 6-, 7-, or 8-position of the isoquinoline ring); with R⁶-R⁸ as defined above

h is 1, 2, 3 or 4

i is 1-7

G⁶ is selected from the group consisting of C₁-C₆ alkyl, substituted C₁-C₆ alkyl,

An even more preferred embodiment of the invention provides compounds having structural Formula (XXIX) shown above or a salt, hydrate, solvate or N-oxide or a derivative thereof which is a prodrug wherein:

G¹ and G³ are hydrogen;

G² is selected from the group consisting of hydrogen, methyl, Bromine, Chlorine or Flourine

h is 2,

i is 2, 3, or 4

and G⁶ is C₁-C₄ alkyl,

An especially preferred embodiment of the invention provides compounds having the structures shown below or a salt, hydrate, solvate or N-oxide or a derivative thereof which is a prodrug:

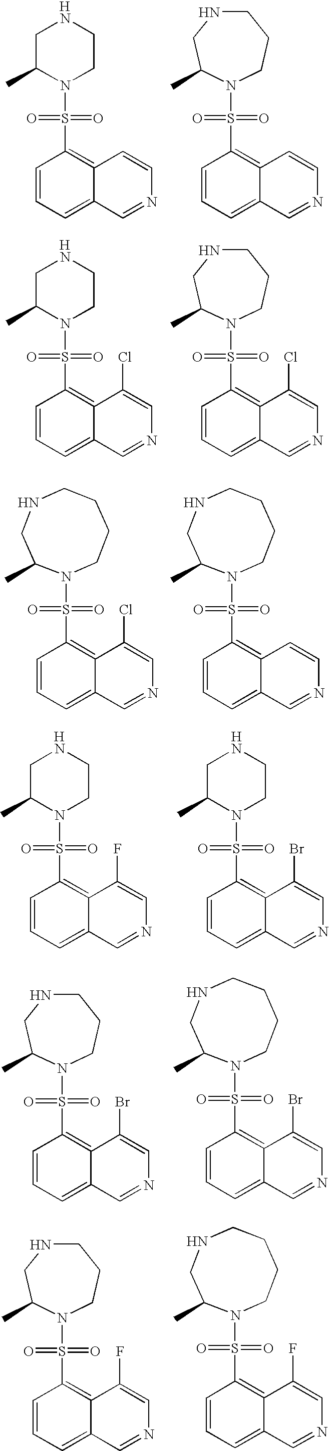

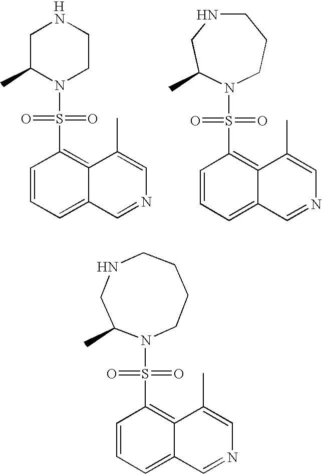

Yet another preferred embodiment of the invention provides compounds having structural Formula (XXVIII) shown above or a salt, hydrate, solvate or N-oxide thereof or a derivative thereof which is a prodrug wherein:

G¹ is selected from the group consisting of hydrogen or hydroxyl

G² is selected from the group consisting of hydrogen, C₁-C₄ alkyl, Bromine, Chlorine, Flourine, C₁-C₄ alkenyl, C₁-C₄ alkynyl

G³ is hydrogen;

G⁴ and G⁵ together with the atoms to which they are bonded form a cycloheteroalkyl or substituted cycloheteroalkyl ring of the formulas (g) and (h) below;

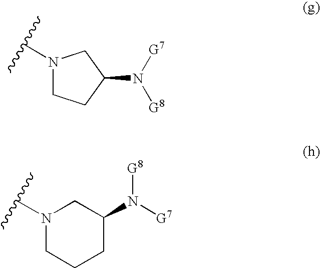

G⁷ and G⁸ are independently selected from the group consisting of hydrogen, alkyl, substituted alkyl, alkenyl, substituted alkenyl heteroalkyl, substituted heteroalkyl, aryl, substituted aryl, heteroaryl, and substituted heteroaryl;

And Z¹ is SO₂

Another especially preferred embodiment of the invention provides compounds having structural Formulas XXX and XXXI shown below:

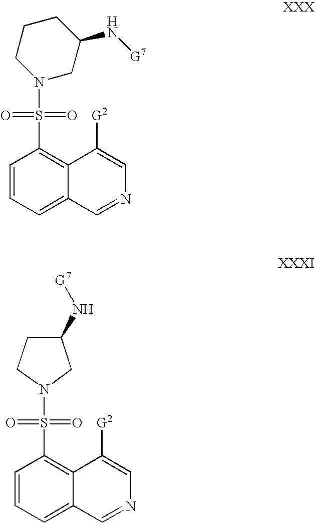

G² is selected from the group consisting of methyl, ethyl, propyl, Bromine, Chlorine, Flourine, C₁-C₄ alkenyl, C₁-C₃ alkynyl;

And G⁷ is selected from the group consisting of C₁-C₃, alkyl, or one of the following groups:

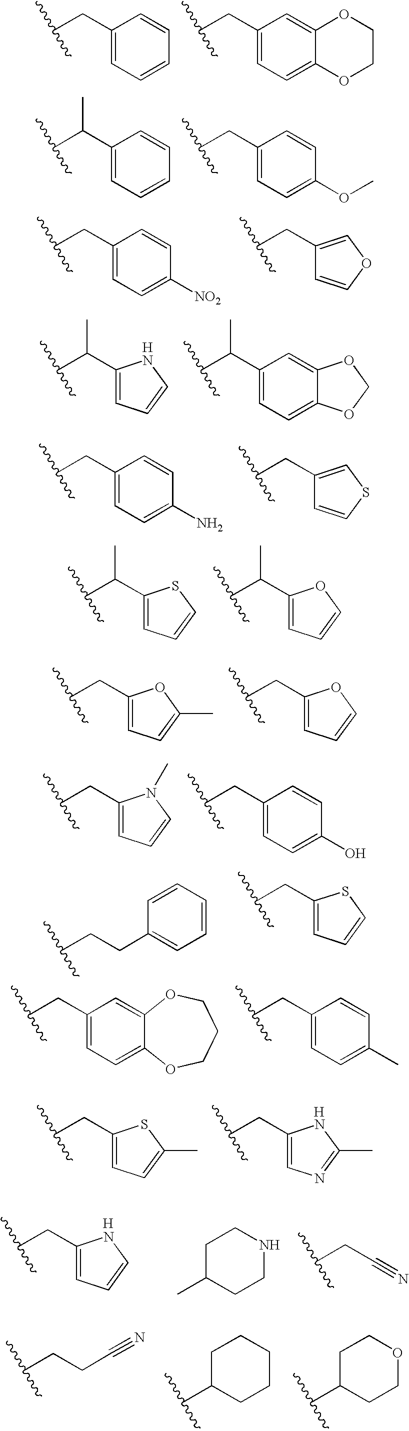

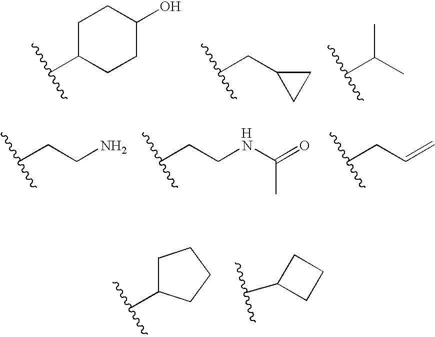

In a second aspect of the invention, a compound of structural Formula (XXXII) is provided:

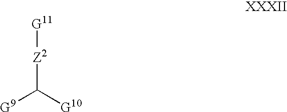

or a salt, hydrate, solvate or N-oxide thereof or a derivative there of which is a prodrug wherein:

G⁹ and G¹⁰ are independently selected from the group consisting of alkyl, substituted alkyl, alkenyl, substituted alkenyl, alkynyl substituted alkynyl, aryl, substituted aryl, heteroalkyl, substituted heteroalkyl, heteroaryl and substituted heteroaryl;

G¹¹ is selected from the group consisting of hydrogen, hydroxyl, alkyl substituted alkyl, aryl, substituted aryl, heteroalkyl, substituted heteroalkyl, heteroaryl, substituted heteroaryl, OR⁶, NR⁶R⁷, NR⁶SO₂R⁷, and NR⁶SO₂NR⁷R⁸;

R⁶-R⁸ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl and substituted heteroarylalkyl or alternatively, R⁶ and R⁷, R⁶ and R⁸, or R⁷ and R⁸, together with the atoms to which they are bonded form a cycloheteroalkyl or substituted cycloheteroalkyl ring;

Z² is selected from SO₂ or C(O)

A preferred embodiment of the invention, provides compounds having structural Formulas (XXXII) shown above or a salt, hydrate, solvate or N-oxide thereof or a derivative thereof which is a prodrug wherein:

G⁹ is selected from the group consisting of aryl, substituted aryl, heteroaryl and substituted heteroaryl;

G¹⁰ is selected from the group consisting of C₁-C₈ heteroalkyl, substituted C₁-C₈ heteroalkyl, C₃-C₈ cycloheteroalkyl, or substituted C₃-C₈ cycloheteroalkyl,

G¹¹ is selected from the group consisting of hydrogen, hydroxyl, C₁-C₈ alkyl, substituted C₁-C₈ alkyl, C₁-C₈ heteroalkyl, substituted C₁-C₈ heteroalkyl, OR⁶, and NR⁶R⁷;

R⁶-R⁸ are independently selected from the group consisting of hydrogen, alkyl, substituted alkyl, heteroalkyl, substituted heteroalkyl,

and Z² is C(O)

An especially preferred embodiment of the invention, provides compounds having structural Formulas (V) shown above or a salt, hydrate, solvate or N-oxide thereof or a derivative thereof which is a prodrug wherein:

G⁹ is selected from the group consisting of phenyl, or substituted phenyl,

G¹⁰ is selected from the group consisting of C₃-C₆ cyclo heteroalkyl ring containing 1 nitrogen atom;

G¹¹ is selected from the group consisting of hydroxl, methoxy, or ethoxy;

Ribavirin (Copegus®; Rebetol®; Ribasphere®; Vilona®, Virazole®, also generics from Sandoz, Teva, Warrick) (1-(β-D-Ribofuranosyl)-1H-1,2,4-triazole-3-carboxamide) is an anti-viral drug having the structure:

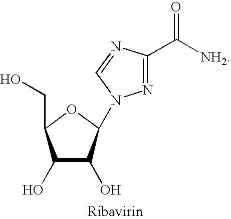

This compounds is active against a number of DNA and RNA viruses. It is a member of the nucleoside antimetabolite drugs that interfere with duplication of viral genetic material. Though not effective against all viruses, ribavirin is remarkable as a small molecule for its wide range of activity, including important activities against influenzas, flaviviruses and agents of many virla hemorrhagic fevers.

Ribavirin is a pro-drug, meaning that it is a chemical precursor for the actual pharmacologically active molecule. When the pro-drug is administered, the body converts it into the desired chemical. Ribavirin is activated by cellular kinases that change it into the 5′ triphosphate nucleotide. In this form it interferes with aspects of RNA metabolism related to viral replication. A number of mechanisms have been proposed for this (see Mechanisms of Action, below) but none of these is proven. More than one mechanism may be active.

In the U.K. & the U.S. the oral (capsule or tablet) form of ribavirin is used in the treatment of hepatitis C, in combination with interferon drugs. The aerosol form is used to treat respiratory syncytial virus-related diseases in children. In Mexico, ribavirin (“ribavirina”) has been sold for use against influenza.

The primary observed serious adverse side-effect of ribavirin is hemolytic anemia, which may worsen preexisting cardiac disease. The mechanism for this effect is unknown. It is dose-dependent and may sometimes be compensated by decreasing dose. Ribavirin is also a teratogen in some animals species and thus poses a theoretical reproductive risk in humans, remaining a hazard as long as the drug is present, which can be as long as 6 months after a course of the drug has ended. In a third aspect of the invention, a compound of structural Formulas (XXXIII) and (XXXIV) is provided:

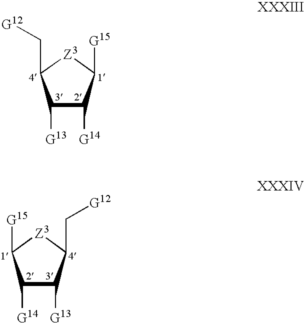

or a salt, hydrate, solvate or N-oxide thereof or a derivative there of which is a prodrug wherein:

Z³ is selected from CH₂, NR⁶, or O; where R⁶ is as defined above

G¹³, G¹⁴, G¹⁵ are independently selected from the group consisting of hydrogen, hydroxyl, halogen, C₁-C₄ alkyl OR⁶, S(O)_{b}R⁶, NR⁶R⁷, CONR⁶R⁷, CO₂R⁶, OCOR⁶, OCONR⁶R⁷, NR⁶CO₂R⁷, NR⁶CONR⁷R⁸, NR⁶CSNR⁷R⁸, NR⁶C(═NH)NR⁷R⁸, SO₂NR⁶R⁷, NR⁶SO₂R⁷, NR⁶SO₂NR⁷R⁸, P(O)(OR⁶)(OR⁷), and P(O)(R⁶)(OR⁷), OP(O)(OR⁶), and OP(O)(R⁶); or together, G¹³ and G¹⁴ can form a double bond between the 2′ and 3′ position within the ring

b=0, 1, or 2

R⁶-R⁸ are independently selected from the group consisting of hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl and substituted heteroarylalkyl or alternatively, R⁶ and R⁷, R⁶ and R⁸, or R⁷ and R⁸, together with the atoms to which they are bonded form a cycloheteroalkyl or substituted cycloheteroalkyl ring;

G¹² is selected from aryl, substituted aryl, heteroaryl, substituted heteroaryl, heteroarylalkyl, substituted heteroarylalkyl cycloheteroalkyl and substituted cycloheteroalkyl;

A preferred embodiment of the invention, provides compounds having structural Formulas (XXXIII or XXXIV) shown above or a salt, hydrate, solvate or N-oxide thereof or a derivative thereof which is a prodrug wherein:

Z³ is selected from CH₂, or O;

G¹³, G¹⁴, G¹⁵ are independently selected from the group consisting of hydrogen, hydroxyl, halogen, OR⁶, or together, G¹³ and G¹⁴ can form a double bond between the 2′ and 3′ position within the ring and R⁶ is as defined above
G¹² is selected from heteroaryl, substituted heteroaryl, heteroarylalkyl and substituted heteroarylalkyl cycloheteroalkyl or substituted cycloheteroalkyl;

An even more preferred embodiment of the invention, provides compounds having structural Formulas (XXXIII or XXXIV) shown above or a salt, hydrate, solvate or N-oxide thereof or a derivative thereof which is a prodrug wherein:

Z³ is selected from CH₂, or O;

G¹³, G¹⁴, G¹⁵ are, hydroxyl; In some instances G¹³, G¹⁴, G¹⁵ may be alkoxyl aryloxy or heteroaryloxy, alkoxycarbonyl, substituted alkoxycarbonyl, carbamoyl and substituted carbamoyl or a hydroxyl that has been otherwise modified by an organic radical that can be removed under physiological conditions such that the cleavage products are physiologically tolerable at the resulting concentrations;

G¹² is selected from:

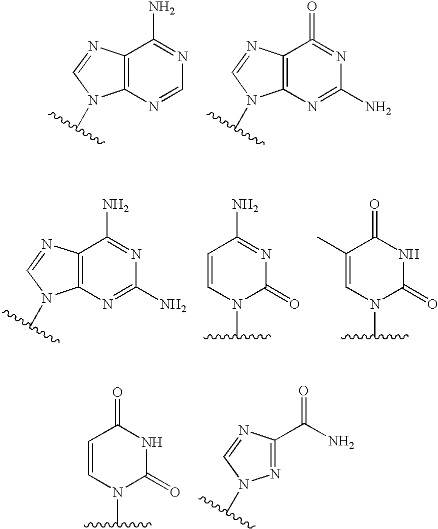

Another more preferred embodiment of the invention, provides compounds having structural Formulas (XXXIII or XXXIV) shown above or a salt, hydrate, solvate or N-oxide thereof or a derivative thereof which is a prodrug wherein:

Z³ is selected from CH₂, or O;

G¹⁵ is hydroxyl, or in some instances G¹⁵ may be alkoxyl aryloxy or heteroaryloxy, alkoxycarbonyl, substituted alkoxycarbonyl, carbamoyl and substituted carbamoyl or a hydroxyl that has been otherwise modified by an organic radical that can be removed under physiological conditions such that the cleavage products are physiologically tolerable at the resulting concentrations;

Together, G¹³ and G¹⁴ form a double bond between the 2′ and 3′ position within the ring

and G¹² is selected from:

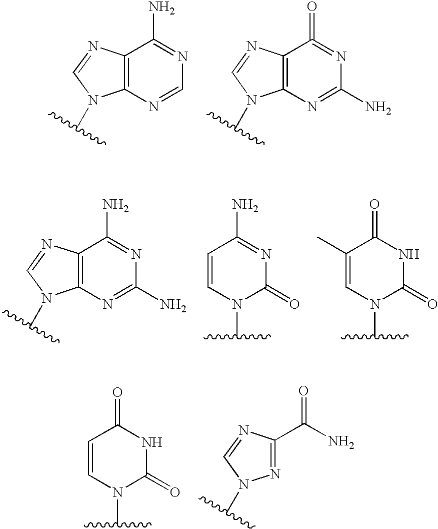

Another especially preferred embodiment of the invention provides compounds having the following formula:

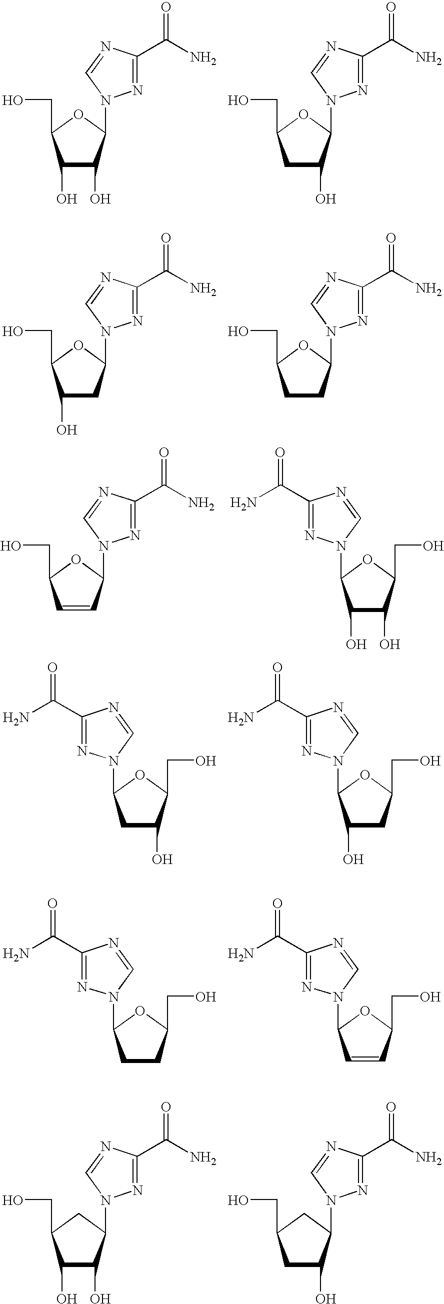

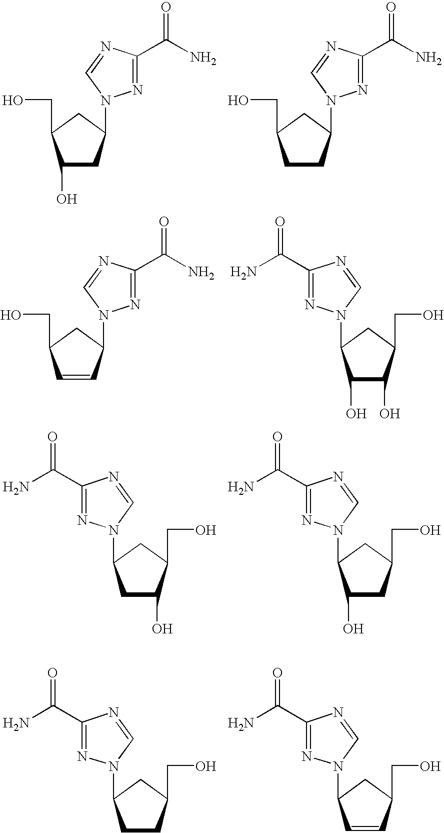

Yohimbine (17α-hydroxy-yohimban-16α-carboxylic acid methyl ester) has the following structure:

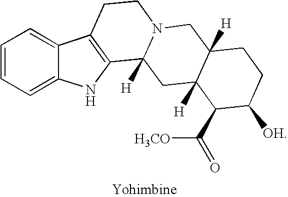

Yohimbine is a selective competitive alpha 2-adrenergic receptor antagonist. The alpha2 receptor is responsible for sensing adrenaline and noradrenaline and telling the body to decrease its production as part of a negative feedback loop. Yohimbine also antagonizes several serotonin receptor subtypes: 1A (inhibitory, behavioral control), 1B (inhibitory, vasoconstriction), 1D (inhibitory, vasoconstriction), and 2B (smooth muscle contraction). Since yohimbine is an antagonist, it will decrease the effects of these receptors, thus causing excitation, vasodilation, and smooth muscle relaxation. Yohimbine is also said to increase dopamine and have some actions as an MAOI, although these mechanisms are unknown.

In some embodiments of the disclosure, a renin inhibitor in combination with one or more neurogenic agents as described herein, is in the form of a composition that includes at least one pharmaceutically acceptable excipient. As used herein, the term “pharmaceutically acceptable excipient” includes any excipient known in the field as suitable for pharmaceutical application. Suitable pharmaceutical excipients and formulations are known in the art and are described, for example, in Remington's Pharmaceutical Sciences (19th ed.) (Genarro, ed. (1995) Mack Publishing Co., Easton, Pa.). Pharmaceutical carriers may be chosen based upon the intended mode of administration of a renin inhibitor in combination with one or more neurogenic agents as described herein. The pharmaceutically acceptable carrier may include, for example, disintegrants, binders, lubricants, glidants, emollients, humectants, thickeners, silicones, flavoring agents, and water.

A renin inhibitor in combination with one or more neurogenic agents as described herein may be incorporated with excipients and administered in the form of ingestible tablets, buccal tablets, troches, capsules, elixirs, suspensions, syrups, wafers, or any other form known in the pharmaceutical arts. The pharmaceutical compositions may also be formulated in a sustained release form. Sustained release compositions, enteric coatings, and the like are known in the art. Alternatively, the compositions may be a quick release formulation.

The amount of a combination of a renin inhibitor in combination with one or more neurogenic agents as described herein, may be an amount that also potentiates or sensitizes, such as by activating or inducing cells to differentiate, a population of neural cells for neurogenesis. The degree of potentiation or sensitization for neurogenesis may be determined with use of the combination in any appropriate neurogenesis assay, including, but not limited to, a neuronal differentiation assay described herein. In some embodiments, the amount of a combination of a renin inhibitor in combination with one or more neurogenic agents as described herein, is based on the highest amount of one agent in a combination, which amount produces no detectable neuroproliferation in vitro but yet produces neurogenesis, or a measurable shift in efficacy in promoting neurogenesis in vitro, when used in the combination.

As disclosed herein, an effective amount of a renin inhibitor in combination with one or more neurogenic agents as described herein, in the described methods is an amount sufficient, when used as described herein, to stimulate or increase neurogenesis in the subject targeted for treatment when compared to the absence of the combination. An effective amount of a renin inhibitor in combination with one or more neurogenic agents as described herein may vary based on a variety of factors, including but not limited to, the activity of the active compounds, the physiological characteristics of the subject, the nature of the condition to be treated, and the route and/or method of administration. General dosage ranges of certain compounds are provided herein and in the cited references based on animal models of CNS diseases and conditions. Various conversion factors, formulas, and methods for determining human dose equivalents of animal dosages are known in the art, and are described, e.g., in Freireich et al., Cancer Chemother Repts 50(4): 219 (1966), Monro et al., Toxicology Pathology, 23: 187-98 (1995), Boxenbaum and Dilea, J. Clin. Pharmacol. 35: 957-966 (1995), and Voisin et al., Reg. Toxicol. Pharmacol., 12(2): 107-116 (1990), which are herein incorporated by reference.

The disclosed methods typically involve the administration of a renin inhibitor in combination with one or more neurogenic agents as described herein, in a dosage range of from about 0.001 ng/kg/day to about 200 mg/kg/day. Other non-limiting dosages include from about 0.001 to about 0.01 ng/kg/day, about 0.01 to about 0.1 ng/kg/day, about 0.1 to about 1 ng/kg/day, about 1 to about 10 ng/kg/day, about 10 to about 100 ng/kg/day, about 100 ng/kg/day to about 1 μg/kg/day, about 1 to about 2 μg/kg/day, about 2 μg/kg/day to about 0.02 mg/kg/day, about 0.02 to about 0.2 mg/kg/day, about 0.2 to about 2 mg/kg/day, about 2 to about 20 mg/kg/day, or about 20 to about 200 mg/kg/day. However, as understood by those skilled in the art, the exact dosage of a renin inhibitor in combination with one or more neurogenic agents as described herein, used to treat a particular condition will vary in practice due to a wide variety of factors. Accordingly, dosage guidelines provided herein are not limiting as the range of actual dosages, but rather provide guidance to skilled practitioners in selecting dosages useful in the empirical determination of dosages for individual patients. Advantageously, methods described herein allow treatment of one or more conditions with reductions in side effects, dosage levels, dosage frequency, treatment duration, safety, tolerability, and/or other factors. Suitable dosages for a renin inhibitor in combination with one or more neurogenic agents as described herein for other indications are known to a skilled person. The disclosure includes the use of about 75%, about 50%, about 33%, about 25%, about 20%, about 15%, about 10%, about 5%, about 2.5%, about 1%, about 0.5%, about 0.25%, about 0.2%, about 0.1%, about 0.05%, about 0.025%, about 0.02%, about 0.01%, or less than the known dosage.

In other embodiments, the amount of a renin inhibitor in combination with one or more neurogenic agents as described herein used in vivo may be about 50%, about 45%, about 40%, about 35%, about 30%, about 25%, about 20%, about 18%, about 16%, about 14%, about 12%, about 10%, about 8%, about 6%, about 4%, about 2%, or about 1% or less than the maximum tolerated dose for a subject, including where one or more neurogenic agents is used in combination with a renin inhibitor. This is readily determined for each neurogenic agent that has been in clinical use or testing, such as in humans.

Alternatively, the amount of a renin inhibitor in combination with one or more neurogenic agents as described herein, may be an amount selected to be effective to produce an improvement in a treated subject based on detectable neurogenesis in vitro as described above. With a renin inhibitor in combination with one or more neurogenic agents as described herein, the amount is one that minimizes clinical side effects seen with administration of the agent to a subject. The amount of an agent used in vivo may be about 50%, about 45%, about 40%, about 35%, about 30%, about 25%, about 20%, about 18%, about 16%, about 14%, about 12%, about 10%, about 8%, about 6%, about 4%, about 2%, or about 1% or less of the maximum tolerated dose in terms of acceptable side effects for a subject. This is readily determined for the renin inhibitor in combination with one or more neurogenic agents as described herein, as well as those that have been in clinical use or testing, such as in humans.

In other embodiments, the amount of an additional neurogenic sensitizing agent in a combination with a renin inhibitor in combination with one or more neurogenic agents as described herein may be the highest amount which produces no detectable neurogenesis in vitro, including in animal (or non-human) models for behavior linked to neurogenesis, but yet produces neurogenesis, or a measurable shift in efficacy in promoting neurogenesis in the in vitro assay, when used in combination with a renin inhibitor. Embodiments include amounts which produce about 1%, about 2%, about 4%, about 6%, about 8%, about 10%, about 12%, about 14%, about 16%, about 18%, about 20%, about 25%, about 30%, about 35%, or about 40% or more of the neurogenesis seen with the amount that produces the highest level of neurogenesis in an in vitro assay.

In some embodiments, the amount may be the lowest needed to produce a desired, or minimum, level of detectable neurogenesis or beneficial effect. Of course, the administered renin inhibitor in combination with one or more neurogenic agents as described herein may be in the form of a pharmaceutical composition.

As described herein, the amount of a renin inhibitor in combination with one or more neurogenic agents as described herein, may be any that is effective to produce neurogenesis.

In some embodiments, the amount may be the lowest needed to produce a desired, or minimum, level of detectable neurogenesis or beneficial effect. The administered renin inhibitor in combination with one or more neurogenic agents as described herein may be in the form of a pharmaceutical composition.

In some embodiments, an effective, neurogenesis modulating amount of a renin inhibitor in combination with one or more neurogenic agents as described herein, is an amount that achieves a concentration within the target tissue, using the particular mode of administration, at or above the IC₅₀ or EC₅₀ for activity of target molecule or physiological process. In some cases, a renin inhibitor in combination with one or more neurogenic agents as described herein, may be administered in a manner and dosage that gives a peak concentration of about 1, about 1.5, about 2, about 2.5, about 5, about 10, about 20 or more times the IC₅₀ or EC₅₀ concentration of the renin inhibitor in combination with one or more neurogenic agents as described herein. IC₅₀ and EC₅₀ values and bioavailability data for renin inhibitors and neurogenic agents are known in the art, and are described, e.g., in the references cited herein or may be readily determined using established methods. In addition, methods for determining the concentration of a free compound in plasma and extracellular fluids in the CNS, as well pharmacokinetic properties, are known in the art, and are described, e.g., in de Lange et al., AAPS Journal, 7(3): 532-543 (2005). In some embodiments, a renin inhibitor in combination with one or more neurogenic agents as described herein may be administered as a combination or separate agents used together, at a frequency of at least about once daily, or about twice daily, or about three or more times daily, and for a duration of at least about 3 days, about 5 days, about 7 days, about 10 days, about 14 days, or about 21 days, or about 4 weeks, or about 2 months, or about 4 months, or about 6 months, or about 8 months, or about 10 months, or about 1 year, or about 2 years, or about 4 years, or about 6 years or longer.

In other embodiments, an effective, neurogenesis modulating amount of a renin inhibitor in combination with one or more neurogenic agents as described herein is a dose that produces a concentration in an organ, tissue, cell, and/or other region of interest that includes the ED₅₀ (the pharmacologically effective dose in 50% of subjects) with little or no toxicity. IC₅₀ and EC₅₀ values for the modulation of neurogenesis may be determined using methods described in U.S. Provisional Application No. 60/697,905 to Barlow et al., filed Jul. 8, 2005, incorporated by reference, or by other methods known in the art. In some embodiments, the IC₅₀ or EC₅₀ concentration for the modulation of neurogenesis is substantially lower than the IC₅₀ or EC₅₀ concentration for activity of a renin inhibitor in combination with one or more neurogenic agents as described herein at non-targeted molecules and/or physiological processes.

In some methods, the application of a renin inhibitor in combination with one or more neurogenic agents as described herein may allow effective treatment with substantially fewer and/or less severe side effects compared to existing treatments. In some embodiments, combination therapy with a renin inhibitor in combination with one or more neurogenic agents as described herein allows the combination to be administered at dosages that would be sub-therapeutic when administered individually or when compared to other treatments. In other embodiments, each agent in a combination of agents may be present in an amount that results in fewer and/or less severe side effects than that which occurs with a larger amount. Thus, the combined effect of the neurogenic agents will provide a desired neurogenic activity while exhibiting fewer and/or less severe side effects overall. In further embodiments, the disclosed methods allow treatment of certain conditions for which treatment with the same or similar compounds is ineffective using known methods due, for example, to dose-limiting side effects, toxicity, and/or other factors.

As described, the methods of the disclosure comprise contacting a cell with a renin inhibitor in combination with one or more neurogenic agents as described herein, or administering such an agent or combination to a subject, to result in neurogenesis.

In other embodiments, a combination of two or more agents, such as a renin inhibitor in combination with one or more neurogenic agents as described herein may be used.

In some embodiments, methods of treatment disclosed herein comprise the step of administering a renin inhibitor in combination with one or more neurogenic agents as described herein to a mammal, for a time and at a concentration sufficient to treat the condition targeted for treatment. The disclosed methods may be applied to individuals having, or who are likely to develop, disorders relating to neural degeneration, neural damage and/or neural demyelination.

Depending on the desired clinical result, the disclosed agents or pharmaceutical compositions are administered by any means suitable for achieving a desired effect. Various delivery methods are known in the art and may be used to deliver an agent to a subject or to NSCs or progenitor cells within a tissue of interest. The delivery method will depend on factors such as the tissue of interest, the nature of the compound (e.g., its stability and ability to cross the blood-brain barrier), and the duration of the experiment or treatment, among other factors. For example, an osmotic minipump may be implanted into a neurogenic region, such as the lateral ventricle. Alternatively, compounds may be administered by direct injection into the cerebrospinal fluid of the brain or spinal column, or into the eye. Compounds can also be administered into the periphery (such as by intravenous or subcutaneous injection, or oral delivery), and subsequently cross the blood-brain barrier.

In some embodiments, the disclosed agents or pharmaceutical compositions are administered in a manner that allows them to contact the subventricular zone (SVZ) of the lateral ventricles and/or the dentate gyrus of the hippocampus. The delivery or targeting of a renin inhibitor in combination with one or more neurogenic agents as described herein, to a neurogenic region, such as the dentate gyrus or the subventricular zone, may enhances efficacy and reduces side effects compared to known methods involving administration with the same or similar compounds. Examples of routes of administration include parenteral, e.g., intravenous, intradermal, subcutaneous, oral (e.g., inhalation), transdermal (topical), transmucosal, and rectal administration. Intranasal administration generally includes, but is not limited to, inhalation of aerosol suspensions for delivery of compositions to the nasal mucosa, tracheand bronchioli.

In other embodiments, a renin inhibitor in combination with one or more neurogenic agents as described herein may be administered so as to either pass through or by-pass the blood-brain barrier. Methods for allowing factors to pass through the blood-brain barrier are known in the art, and include minimizing the size of the factor, providing hydrophobic factors which facilitate passage, and conjugation to a carrier molecule that has substantial permeability across the blood brain barrier. In some instances, an agent or combination of agents may be administered by a surgical procedure implanting a catheter coupled to a pump device. The pump device can also be implanted or be extracorporally positioned. Administration of a renin inhibitor in combination with one or more neurogenic agents as described herein may be in intermittent pulses or as a continuous infusion. Devices for injection to discrete areas of the brain are known in the art. In certain embodiments, the combination may be administered locally to the ventricle of the brain, substantia nigra, striatum, locus ceruleous, nucleus basalis Meynert, pedunculopontine nucleus, cerebral cortex, and/or spinal cord by, e.g., injection. Methods, compositions, and devices for delivering therapeutics, including therapeutics for the treatment of diseases and conditions of the CNS and PNS, are known in the art.

In some embodiments, a renin inhibitor in combination with one or more neurogenic agents as described herein may be modified to facilitate crossing of the gut epithelium. For example, in some embodiments, a renin inhibitor in combination with one or more neurogenic agents as described herein in a prodrug form is transported across the intestinal epithelium and metabolized into the active agent in systemic circulation and/or in the CNS.

In other embodiments, a renin inhibitor in combination with one or more neurogenic agents as described herein may be conjugated to a targeting domain to form a chimeric therapeutic, where the targeting domain facilitates passage of the blood-brain barrier (as described above) and/or binds one or more molecular targets in the CNS. In some embodiments, the targeting domain binds a target that is differentially expressed or displayed on, or in close proximity to, tissues, organs, and/or cells of interest. In some cases, the target is distributed in a neurogenic region of the brain, such as the dentate gyrus and/or the SVZ. For example, in some embodiments, a renin inhibitor in combination with one or more neurogenic agents as described herein may be conjugated or complexed with the fatty acid docosahexaenoic acid (DHA), which is readily transported across the blood brain barrier and imported into cells of the CNS.

Of course a further combination therapy may also be that of a renin inhibitor in combination with one or more neurogenic agents, with a non-chemical based therapy. Non-limiting examples include the use of psychotherapy for the treatment of many conditions described herein, such as the psychiatric conditions, as well as behavior modification therapy such as that use in connection with a weight loss program.

Having now generally described the disclosure, the same will be more readily understood through reference to the following examples which are provided by way of illustration, and are not intended to be limiting to the disclosure, unless otherwise specified.

### EXAMPLES

### Example 1

### Effects of the Renin Inhibitor Aliskiren in Combination with 5-HT3 Receptor Antagonists on Neuronal Differentiation of Human Neural Stem Cells

Human neural stem cells (hNSCs) were isolated and grown in monolayer culture, plated, treated with varying concentrations of aliskiren in the presence or absence of a 5-HT3 receptor antagonist, and stained with TUJ-1 antibody for the detection of neuronal differentiation or GFAP antibody for the detection of astrocyte differentiation, as described in U.S. Patent Publication 2007/0015138 (incorporated by reference). Mitogen-free test media with a positive control for neuronal differentiation was used along with basal media without growth factors as a negative control.

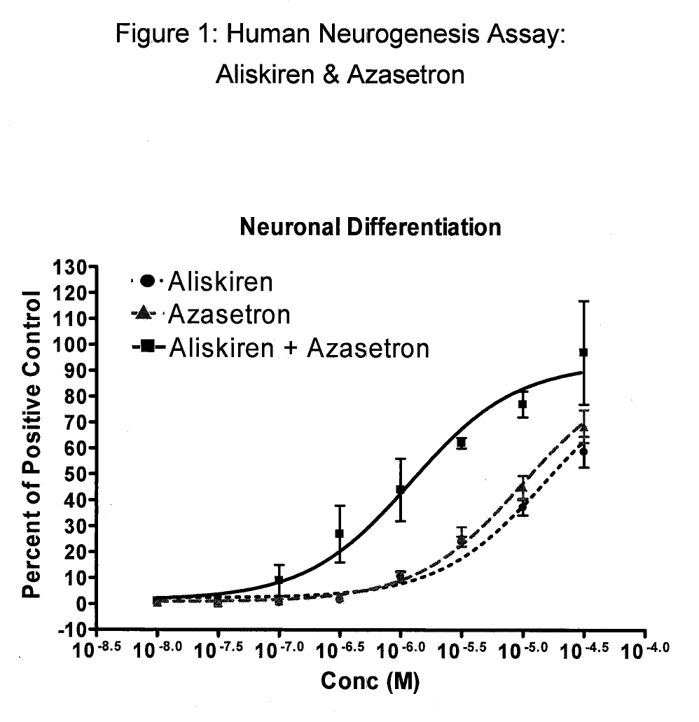

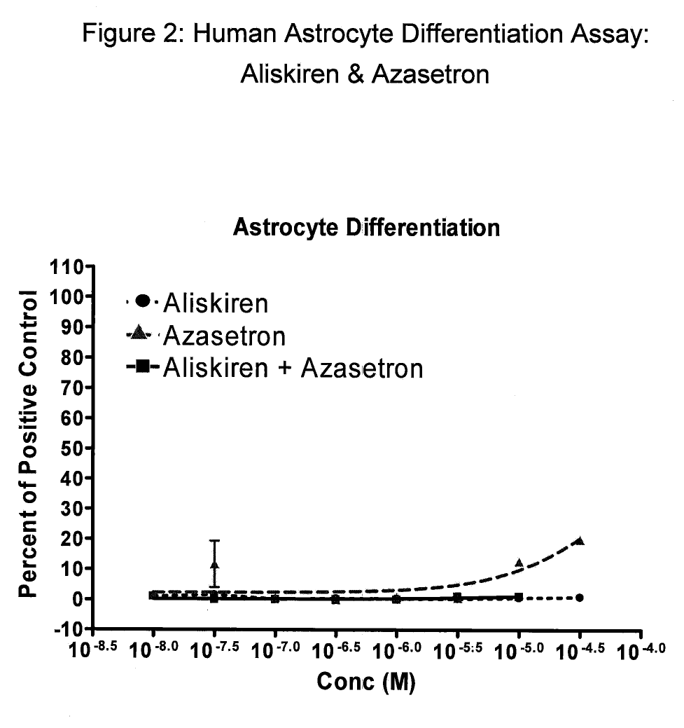

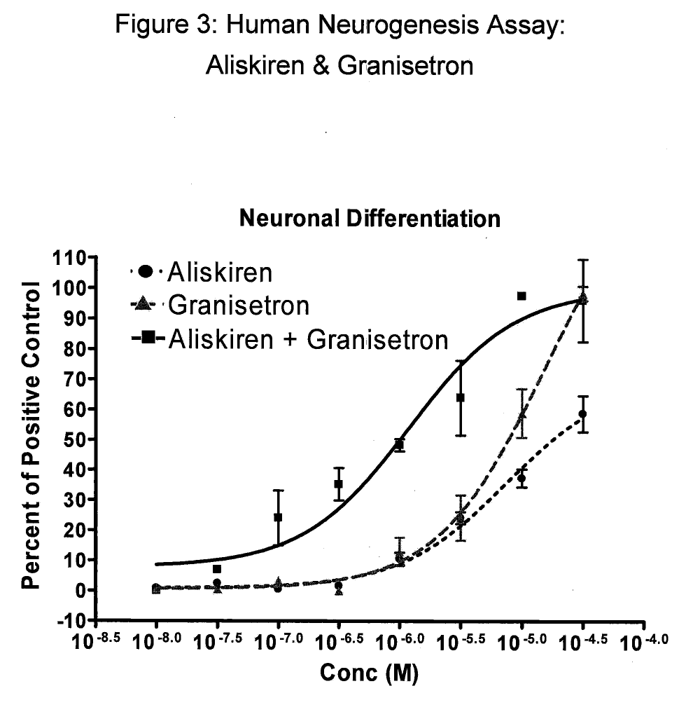

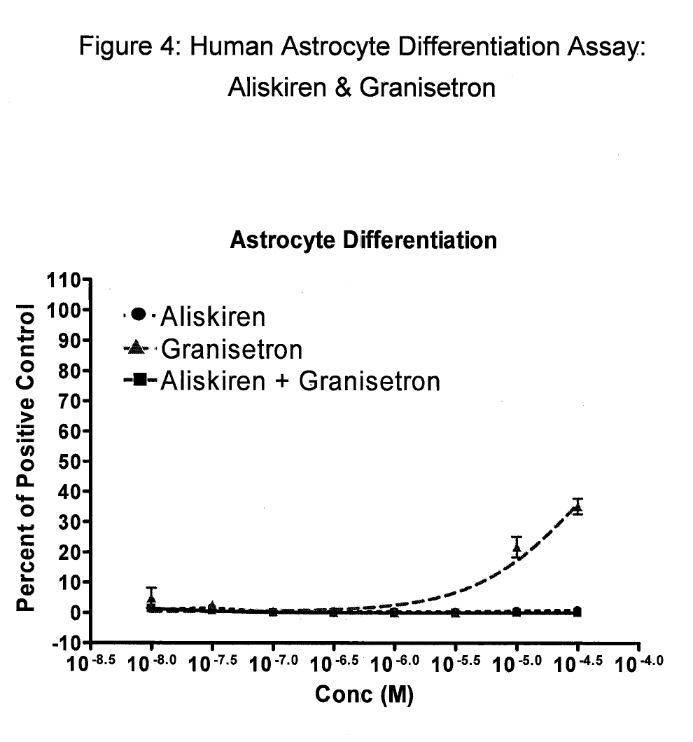

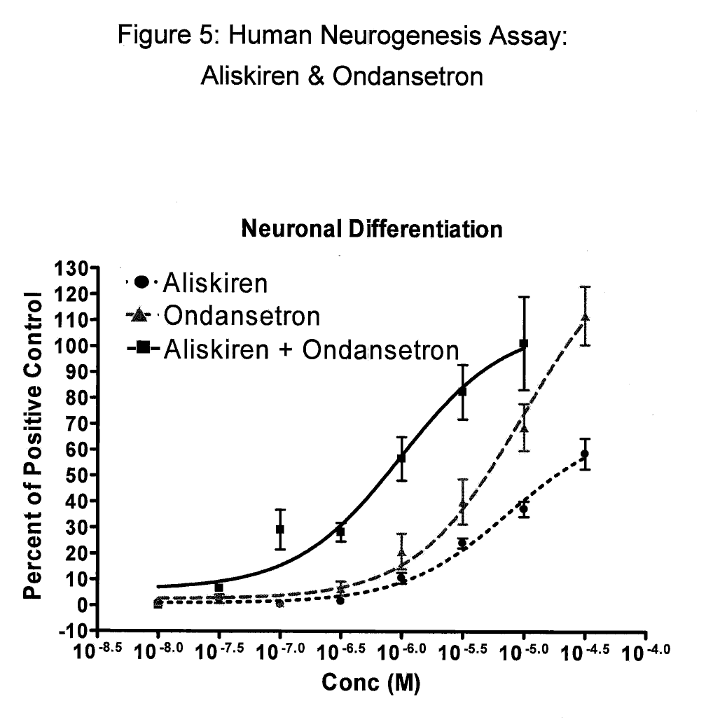

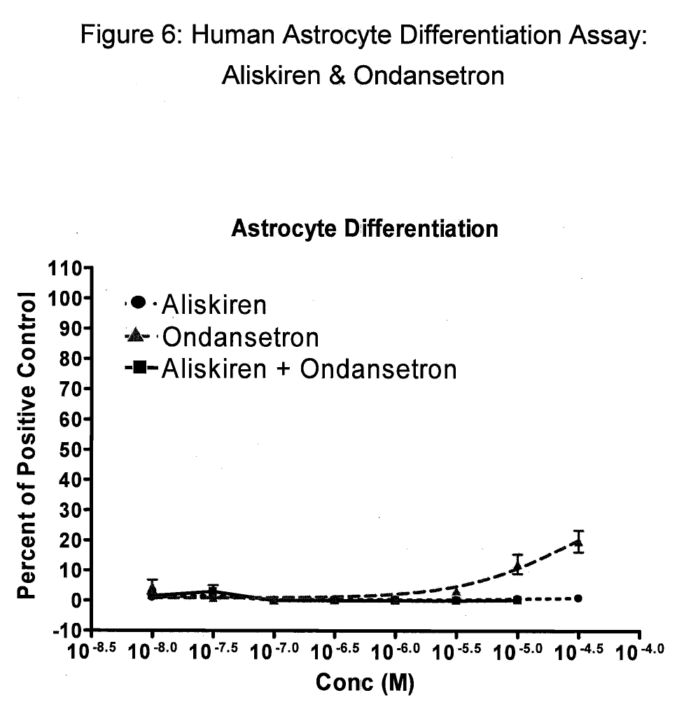

Results are shown in FIGS. 1, 2, 3, 4, 5 and 6, which show concentration response curves of neuronal or astrocyte differentiation, after subtraction of background media values. The concentration response curves of the combination of aliskiren with a 5-HT3 receptor antagonist (azasetron in FIGS. 1 and 2, granisetron in FIGS. 3 and 4, and ondansetron in FIGS. 5 and 6) are shown with the concentration response curves of aliskiren or the 5-HT3 receptor antagonist alone. The data is presented as a percent of neuronal or astrocyte positive control. The data indicate that the combination of aliskiren with a 5-HT3 receptor antagonist resulted in a synergistic increase in neuronal differentiation with a simultaneous decrease in astrocyte differentiation, promoting significantly increased differentiation of human neural stem cells specifically into a neuronal fate.

### Example 2

### Effects of the Renin Inhibitor Aliskiren in Combination with the Anti-Viral Agent Ribavirin on Neuronal Differentiation of Human Neural Stem Cells

Human neural stem cells (hNSCs) were isolated and grown in monolayer culture, plated, treated with varying concentrations of aliskiren in the presence or absence of ribavirin, and stained with TUJ-1 antibody for the detection of neuronal differentiation or GFAP antibody for the detection of astrocyte differentiation, as described in U.S. Patent Publication 2007/0015138 (incorporated by reference). Mitogen-free test media with a positive control for neuronal differentiation was used along with basal media without growth factors as a negative control.

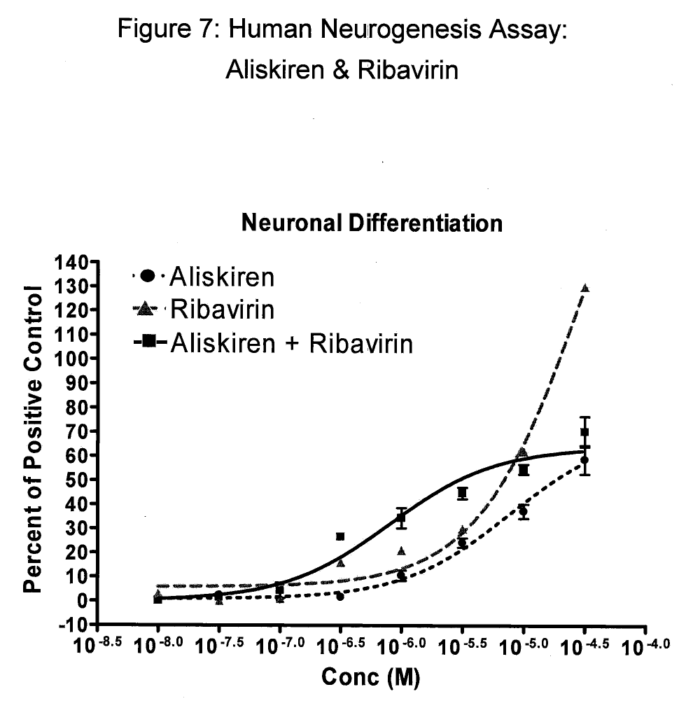

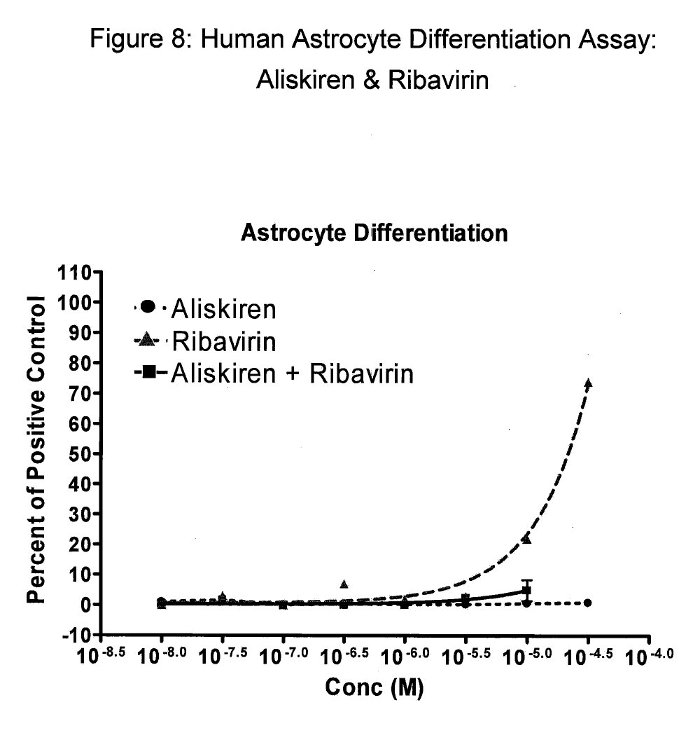

Results are shown in FIGS. 7 and 8, which show concentration response curves of neuronal or astrocyte differentiation, respectively, after subtraction of background media values. The concentration response curves of the combination of aliskiren with ribavirin are shown with the concentration response curves of aliskiren or the ribavirin alone. The data is presented as a percent of neuronal or astrocyte positive control. The data indicate that the combination of aliskiren with ribavirin resulted in a synergistic increase in neuronal differentiation with a simultaneous decrease in astrocyte differentiation, promoting a significantly increased differentiation of human neural stem cells specifically into a neuronal fate.

### Example 3

### Effects of the Renin Inhibitor Aliskiren in Combination with the Dopamine Modulator Methylphenidate on Neuronal Differentiation of Human Neural Stem Cells

Human neural stem cells (hNSCs) were isolated and grown in monolayer culture, plated, treated with varying concentrations of aliskiren in the presence or absence of methylphenidate, and stained with TUJ-1 antibody for the detection of neuronal differentiation, as described in U.S. Patent Publication 2007/0015138 (incorporated by reference). Mitogen-free test media with a positive control for neuronal differentiation was used along with basal media without growth factors as a negative control.

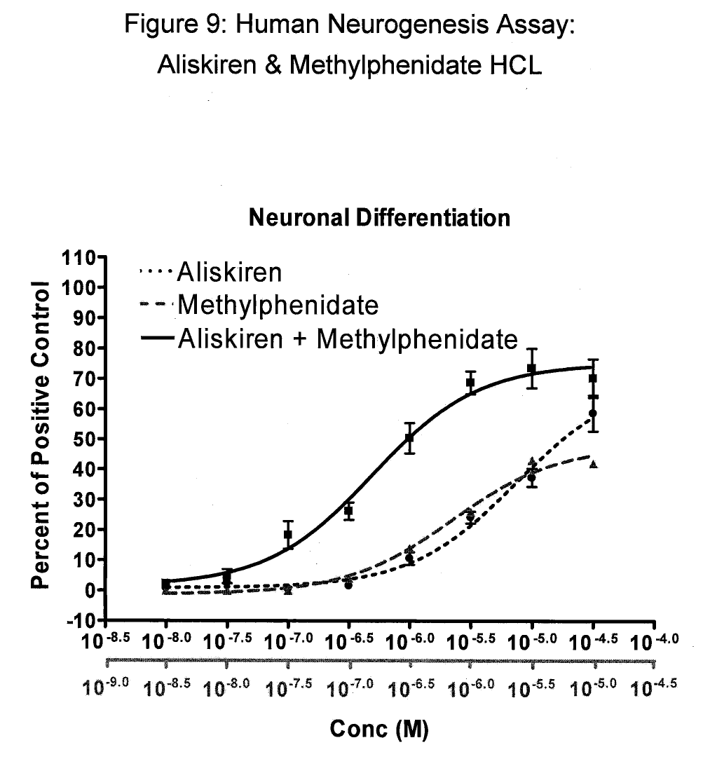

Results are shown in FIG. 9, which shows concentration response curves of neuronal differentiation after subtraction of background media values. The concentration response curves of the combination of aliskiren with methylphenidate are shown with the concentration response curves of each agent alone. The data is presented as a percent of neuronal positive control. The data indicate that the combination of aliskiren with methylphenidate resulted in synergistically enhanced neuronal differentiation relative to that produced by either agent alone.

### Example 4

### Effects of the Renin Inhibitor Aliskiren in Combination with the α2-Adrenergic Receptor Antagonist Yohimbine on Neuronal Differentiation of Human Neural Stem Cells

Human neural stem cells (hNSCs) were isolated and grown in monolayer culture, plated, treated with varying concentrations of aliskiren in the presence or absence of yohimbine, and stained with TUJ-1 antibody for the detection of neuronal differentiation, as described in U.S. Patent Publication 2007/0015138 (incorporated by reference). Mitogen-free test media with a positive control for neuronal differentiation was used along with basal media without growth factors as a negative control.

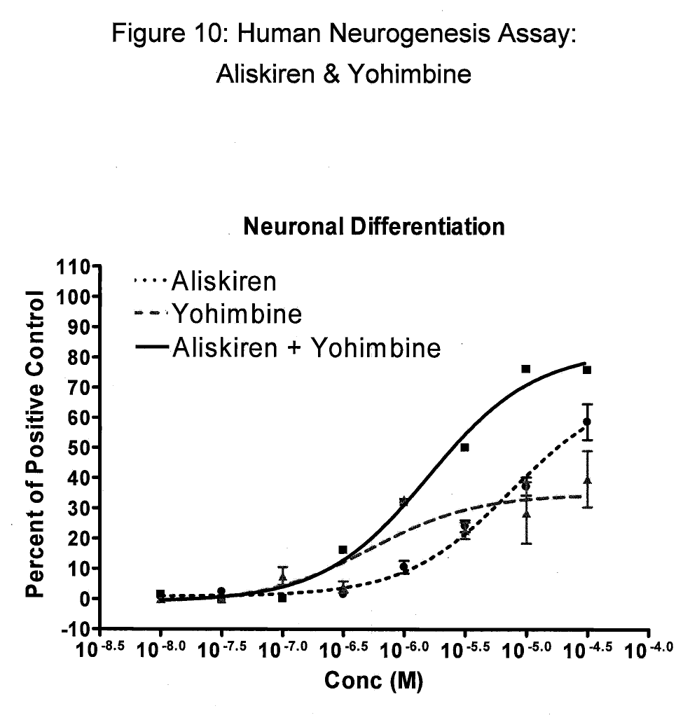

Results are shown in FIG. 10, which shows concentration response curves of neuronal differentiation after subtraction of background media values. The concentration response curves of the combination of aliskiren with yohimbine are shown with the concentration response curves of each agent alone. The data is presented as a percent of neuronal positive control. The data indicate that the combination of aliskiren with yohimbine resulted in synergistically enhanced neuronal differentiation relative to that produced by either agent alone.

### Example 5

### Effects of the Renin Inhibitor Aliskiren in Combination with the Rho Kinase Inhibitor Fasudil on Neuronal Differentiation Of Human Neural Stem Cells

Human neural stem cells (hNSCs) were isolated and grown in monolayer culture, plated, treated with varying concentrations of aliskiren in the presence or absence of fasudil, and stained with TUJ-1 antibody for the detection of neuronal differentiation, as described in U.S. Patent Publication 2007/0015138 (incorporated by reference). Mitogen-free test media with a positive control for neuronal differentiation was used along with basal media without growth factors as a negative control.

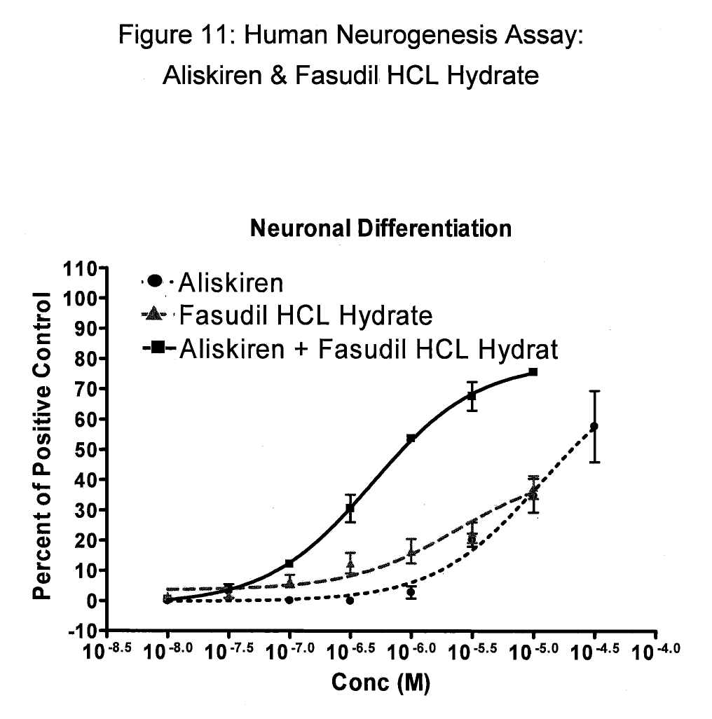

Results are shown in FIG. 11, which shows concentration response curves of neuronal differentiation after subtraction of background media values. The concentration response curves of the combination of aliskiren with fasudil are shown with the concentration response curves of each agent alone. The data is presented as a percent of neuronal positive control. The data indicate that the combination of aliskiren with fasudil resulted in synergistically enhanced neuronal differentiation relative to that produced by either agent alone.

### Example 6

### Effects of the Renin Inhibitor Aliskiren in Combination with the PDE Inhibitor Ibudilast on Neuronal Differentiation of Human Neural Stem Cells

Human neural stem cells (hNSCs) were isolated and grown in monolayer culture, plated, treated with varying concentrations of aliskiren in the presence or absence of ibudilast, and stained with TUJ-1 antibody for the detection of neuronal differentiation, as described in U.S. Patent Publication 2007/0015138 (incorporated by reference). Mitogen-free test media with a positive control for neuronal differentiation was used along with basal media without growth factors as a negative control.

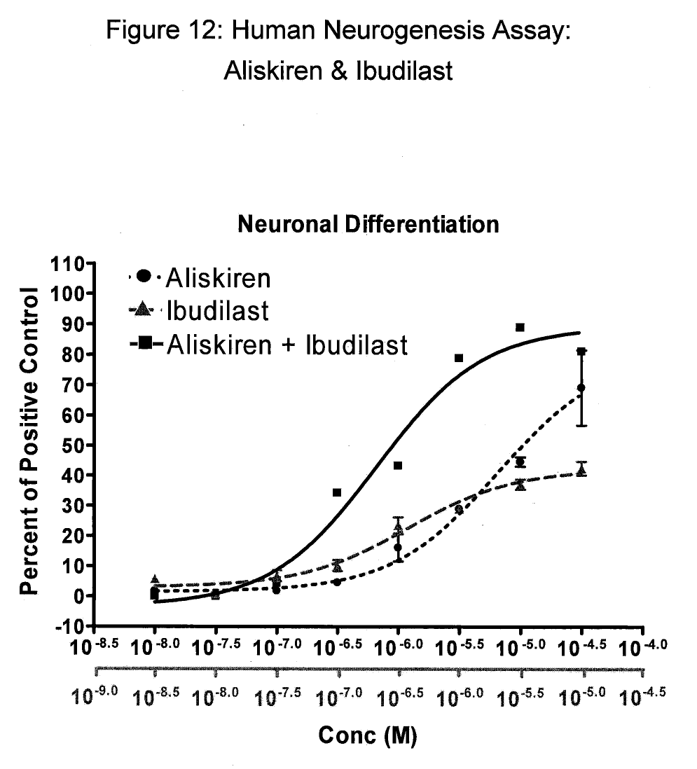

Results are shown in FIG. 12, which shows concentration response curves of neuronal differentiation after subtraction of background media values. The concentration response curves of the combination of aliskiren with ibudilast are shown with the concentration response curves of each agent alone. The data is presented as a percent of neuronal positive control. The data indicate that the combination of aliskiren with ibudilast resulted in synergistically enhanced neuronal differentiation relative to that produced by either agent alone.

All references cited herein, including patents, patent applications, and publications, are hereby incorporated by reference in their entireties, whether previously specifically incorporated or not.

Having now fully provided the instant disclosure, it will be appreciated by those skilled in the art that the same may be performed within a wide range of equivalent parameters, concentrations, and conditions without departing from the spirit and scope of the disclosure and without undue experimentation. While the disclosure has been described in connection with specific embodiments thereof, it will be understood that it is capable of further modifications. This application is intended to cover any variations, uses, or adaptations of the disclosure following, in general, the disclosed principles and including such departures from the disclosure as come within known or customary practice within the art to which the disclosure pertains and as may be applied to the essential features hereinbefore set forth.

What is claimed is:

1. A composition comprising a renin inhibitor in combination with one or more neurogenic agents.

2. The composition of claim 1, wherein the renin inhibitor is aliskiren or a pharmaceutically acceptable salt, hydrate, solvate or N-oxide thereof; and the one or more neurogenic agents is a 5-HT₃ receptor antagonist, a PDE inhibitor, an anti-viral agent, a dopamine modulator, a Rho kinase inhibitor or an alpha2-adrenergic receptor antagonist.

3. The composition of claim 2, wherein the 5-HT₃ receptor antagonist is azasetron, granisetron, ondansetron, or a pharmaceutically acceptable salt, solvate or N-oxide thereof, the PDE inhibitor is ibudilast or a pharmaceutically acceptable salt, solvate or N-oxide thereof, the antiviral agent is ribavirin or a pharmaceutically acceptable salt, hydrate, solvate or N-oxide thereof, the dopamine modulator is methylphenidate or a pharmaceutically acceptable salt, hydrate, solvate or N-oxide thereof; the Rho kinase inhibitor is fasudil or a pharmaceutically acceptable salt, hydrate, solvate or N-oxide thereof; and the alpha2-adrenergic receptor antagonist is yohimbine or a pharmaceutically acceptable salt, solvate or N-oxide thereof.

4. The composition of claim 1, wherein the renin inhibitor in combination with one or more neurogenic agents is in a pharmaceutically acceptable formulation.

5. A method of stimulating or increasing neurogenesis in a cell or tissue, the method comprising contacting the cell or tissue with an effective amount of the composition of claim 1, to stimulate or increase neurogenesis in said cell or tissue.

6. The method of claim 5, wherein the cell or tissue is in an animal or human subject.

7. The method of claim 6, wherein the subject has a condition affecting normal neurogenesis whereby stimulating or increasing neurogenesis improves the condition.

8. A method of claim 7, wherein the condition causes decreased neurogenesis in the subject.

9. A method of claim 7, wherein the condition causes aberrant neurogenesis in the subject.

10. A method of claim 7, wherein the subject had been exposed to an agent causing decreased neurogenesis.

11. A method of claim 5, wherein the neurogenesis comprises differentiation of neural stem cells (NSCs) along a neuronal lineage.

12. A method of claim 5, wherein the neurogenesis comprises differentiation of neural stem cells (NSCs) along a glial lineage.

13. A method of claim 6, wherein the neurogenesis occurs in the hippocampus of the subject.

14. A method of decreasing the level of astrogenesis in a cell or tissue due to an agent that induces or produces astrogenesis, the method comprising contacting the cell or tissue with an effective amount of the composition of claim 1, to decrease the level of astrogenesis in a cell or tissue.

15. The method of claim 14, wherein the agent that induces or produces astrogenesis is also neurogenic.

16. A method claim 14 wherein the cell or tissue is in an animal or human subject.

17. A method of treating a subject suffering from a nervous system disorder comprising administering to the subject an effective amount of the composition of claim 1.

18. A method of claim 17, wherein the subject is an animal or human.

19. A method of claim 17, wherein the nervous system disorder is a mental disorder or a disease of the central nervous system.

20. A method of claim 19, wherein the mental disorder is an affective disorder.

21. A method of claim 19, wherein the disease of the central nervous system causes cognitive impairment.

22. A method of claim 21, wherein the cognitive impairment is the result of chronic infection, toxic disorders, neurodegenerative disorders, or combinations thereof.

23. A method of treating a subject suffering from cognitive impairment due to a non-disease state comprising administering to the subject a therapeutically-effective amount of the composition of claim 1.

24. The method of claim 23, wherein the non-disease state is aging.

25. The method of claim 23, wherein the cognitive impairment is a result of chemotherapy or radiation therapy.
